(19) United States
(12) Patent Application Publication
(10) Pub. No.: US 2020/0385813 A1
(43) Pub. Date: Dec. 10, 2020

(54) SYSTEMS AND METHODS FOR ESTIMATING CELL SOURCE FRACTIONS USING METHYLATION INFORMATION

(71) Applicant: GRAIL, Inc., Menlo Park, CA (US)

(72) Inventor: Oliver Claude Venn, San Francisco, CA (US)

(21) Appl. No.: 16/719,902

(22) Filed: Dec. 18, 2019

Related U.S. Application Data
(60) Provisional application No. 62/781,549, filed on Dec. 18, 2018.

(51) Int. Cl. C12Q 1/6886

(52) U.S. Cl. CPC C12Q 1/6886; C12Q 2600/154; C12Q 2600/112

(57) ABSTRACT

Systems and methods are disclosed for determining a cell source fraction in a biological sample of a test subject. Nucleic acid fragments are obtained from a biological sample, comprising cell-free nucleic acid, of the test subject. A methylation state is obtained for each nucleic acid fragment in a first plurality of nucleic acid fragments. Each respective nucleic acid fragment is individually assigned a first score, thereby obtaining a first plurality of scores. Each respective score represents a likelihood that the corresponding nucleic acid fragment was obtained from a cell-free nucleic acid molecule associated with the first cell source. The first plurality of scores is transformed into a first plurality of counts, each count in the first plurality of counts being for a methylation site in a first predetermined set of methylation sites. A first cell source fraction for the test subject is estimated using the first plurality of counts.

### CROSS REFERENCE TO RELATED APPLICATION

This application claims priority to U.S. Provisional Patent Application No. 62/781,549 entitled “Systems and Methods for Estimating Cell Source Fractions Using Methylation Information,” filed Dec. 18, 2018, which is hereby incorporated by reference.

### TECHNICAL FIELD

This specification describes using nucleic acids, in particular cell-free nucleic acid samples, of a subject to estimate a cell source fractions, such as tumor fraction, in biological samples obtained from a subject.

### BACKGROUND

The increasing knowledge of the molecular basis for cancer and the rapid development of next generation sequencing techniques are advancing the study of early molecular alterations involved in cancer development in body fluids. Large scale sequencing technologies, such as next generation sequencing (NGS), have afforded the opportunity to achieve sequencing at costs that are less than one U.S. dollar per million bases, and in fact costs of less than ten U.S. cents per million bases have been realized. Specific genetic and epigenetic alterations associated with such cancer development are found in plasma, serum, and urine cell-free DNA (cfDNA). Such alterations could potentially be used as diagnostic biomarkers for several classes of cancers (see Salvi et al., 2016, Onco Targets Ther. 9:6549-6559).

Cell-free DNA (cfDNA) can be found in serum, plasma, urine, and other body fluids (Chan et al., 2003, Ann Clin Biochem. 40(Pt 2):122-130) representing a “liquid biopsy,” which is a circulating picture of a specific disease (see De Mattos-Arruda and Caldas, 2016, Mol Oncol. 10(3):464-474). This represents a potential, non-invasive method of screening for a variety of cancers.

The existence of cfDNA was demonstrated by Mandel and Metais decades ago (Mandel and Metais, 1948, C R Seances Soc Biol Fil. 142(3-4):241-243). cfDNA originates from necrotic or apoptotic cells, and it is generally released by all types of cells. Stroun et al further showed that specific cancer alterations could be found in the cfDNA of patients (see, Stroun et al., 1989 Oncology 1989 46(5):318-322). A number of subsequent articles confirmed that cfDNA contains specific tumor-related alterations, such as mutations, methylation, and copy number variations (CNVs), thus confirming the existence of circulating tumor DNA (ctDNA) (see, Goessl et al., 2000 Cancer Res. 60(21):5941-5945 and Frenel et al., 2015, Clin Cancer Res. 21(20):4586-4596).

cfDNA in plasma or serum is well characterized, while urine cfDNA (ucfDNA) has been traditionally less characterized. However, recent studies demonstrated that ucfDNA could also be a promising source of biomarkers (e.g., Casadio et al., 2013, Urol Oncol. 31(8):1744-1750).

In blood, apoptosis is a frequent event that determines the amount of cfDNA. In cancer patients, however, the amount of cfDNA seems to be also influenced by necrosis (see Hao et al., 2014, Br J Cancer 111(8):1482-1489 and Zonta et al., 2015 Adv Clin Chem. 70:197-246). Since apoptosis seems to be the main release mechanism circulating cfDNA has a size distribution that reveals an enrichment in short fragments of about 167 bp, (see, Heitzer et al., 2015, Clin Chem. 61(1):112-123 and Lo et al., 2010, Sci Transl Med. 2(61):61ra91) corresponding to nucleosomes generated by apoptotic cells.

The amount of circulating cfDNA in serum and plasma seems to be significantly higher in patients with tumors than in healthy controls, especially in those with advanced-stage tumors than in early-stage tumors (see, Sozzi et al., 2003, J Clin Oncol. 21(21):3902-3908, Kim et al., 2014, Ann Surg Treat Res. 86(3):136-142; and Shao et al., 2015, Oncol Lett. 10(6):3478-3482). The variability of the amount of circulating cfDNA is higher in cancer patients than in healthy individuals, (see, Heitzer et al., 2013, Int J Cancer. 133(2):346-356) and the amount of circulating cfDNA is influenced by several physiological and pathological conditions, including proinflammatory diseases (see, Raptis and Menard, 1980, J Clin Invest. 66(6):1391-1399, and Shapiro et al., 1983, Cancer 51(11):2116-2120).

Methylation status and other epigenetic modifications are known to be correlated with the presence of some disease conditions such as cancer (see Jones, 2002, Oncogene 21:5358-5360). And specific patterns of methylation have been determined to be associated with particular cancer conditions (see Paska and Hudler, 2015, Biochemia Medica 25(2):161-176). Warton and Samimi have demonstrated that methylation patterns can be observed even in cell-free DNA (Warton and Samimi, 2015, Front Mol Biosci, 2(13) doi: 10.3389/fmolb.2015.00013).

Given the promise of circulating cfDNA, as well as other forms of genotypic data, as a diagnostic indicator, ways of assessing such data for epigenetic patterns are needed in the art.

### SUMMARY

The present disclosure addresses the shortcomings identified in the background by providing systems and methods for determining cell source fractions, such as tumor fraction, in biological samples obtained from a subject using cfDNA. The combination of methylation data with whole genome, or targeted genome, sequencing data provides additional diagnostic power beyond previous screening methods.

Technical solutions (e.g., computing systems, methods, and non-transitory computer readable storage mediums) for addressing the above identified problems with analyzing datasets are provided in the present disclosure.

The following presents a summary of the invention in order to provide a basic understanding of some of the aspects of the invention. This summary is not an extensive overview of the invention. It is not intended to identify key/critical elements of the invention or to delineate the scope of the invention. Its sole purpose is to present some of the concepts of the invention in a simplified form as a prelude to the more detailed description that is presented later.

A. Embodiments that estimate the cell source fraction for at least one cell source by making use of the transformation of nucleic acid fragment scores to methylation counts. One aspect of the present disclosure provides a method of estimating a first cell source fraction in a first biological sample in a test subject of a given species. The method comprises, at a computer system having one or more processors, and memory storing one or more programs for execution by the one or more processors, obtaining a methylation state of each nucleic acid fragment in a first plurality of nucleic acid fragments in electronic form from a first plurality of cell-free nucleic acid molecules in the first biological sample at a first time period. The method further comprises individually assigning a first score to each respective nucleic acid fragment in the first plurality of nucleic acid fragments, thereby obtaining a plurality of first scores. Here, each respective first score represents a likelihood that the corresponding nucleic acid fragment was obtained from a cell-free nucleic acid molecule that originated from the first cell source. Moreover, the individual assignments comprise i) comparing a methylation state of the respective nucleic acid fragment against a first canonical set of methylation state vectors and against a second canonical set of methylation state vectors representative of a source other than the first cell source, or ii) presenting the methylation state of the respective nucleic acid fragment to a first classifier trained at least in part on the first canonical set of methylation state vectors and the second canonical set of methylation state vectors. Each canonical methylation state vector in the first canonical set of methylation state vectors is derived from a respective first tissue sample or a respective first cell-free nucleic acid sample of a corresponding reference subject in a first plurality of reference subjects, where the respective first tissue sample or the respective first cell-free nucleic acid sample corresponds to the first cell source. Each canonical methylation state vector in the second canonical set of methylation state vectors is derived from a respective second tissue sample or a respective second cell-free nucleic acid sample of a corresponding reference subject in a second plurality of reference subjects, where the respective second tissue sample or the respective second cell-free nucleic acid sample corresponds to a second cell source. In some embodiments, the second cell source is a different tissue type or organ type than the first cell source. In some embodiments, the second cell source is the same tissue type or organ type as the first cell source but the first cell source and the second cell source are in different states. As an example of this, in some embodiments the first cell source is colon cells that do not have cancer and the second cell source is colon cells that have cancer. As an example of this, in some embodiments the first cell source is colon cells that have stage I cancer and the second cell source is colon cells that have stage II cancer. In some embodiments, the first cell source is cells from a subject that has a first stage of a particular cancer and the second cell source is cells from a subject that has a second stage of the particular cancer, where the first and second stages of cancer are different. The method further comprises transforming the plurality of first scores into a first plurality of counts. Each count in the first plurality of counts is for a methylation site in a first predetermined set of methylation sites in the genome of a reference sequence of the species. The first predetermined set of methylation sites is associated with the first cell source. The method further comprises estimating a first instance of the first cell source fraction in the first biological sample using the first plurality of counts by comparing the respective count of each respective methylation site in the first predetermined set of methylation sites represented by the first plurality of counts to a corresponding reference score for the respective methylation site in a first reference set. Each corresponding reference score in the first reference set is obtained by determining a frequency of methylation of the corresponding methylation site in nucleic acid fragments obtained from the tissue sample or the cell-free nucleic acid sample of a corresponding reference subject in the first plurality of reference subjects.

In some embodiments, each canonical methylation state vector in the first canonical set of methylation state vectors represents the methylation state across the genome of the corresponding reference subject in the first plurality of reference subjects.

In some embodiments, each canonical methylation state vector in the first canonical set of methylation state vectors represents the methylation state across a subset of the genome of the corresponding reference subject. In such embodiments a methylation state of the subset of the genome is representative of causative biology underlying the first cell source.

In some embodiments, the first cell source is a type of cancer and a canonical methylation state vector in the first canonical set of methylation state vectors is derived from a sample of a tumor of the type of cancer obtained from the corresponding reference subject.

In some embodiments, the first cell source is a type of cancer. Furthermore, a canonical methylation state vector in the first set of canonical methylation state vectors is derived from cell-free nucleic acids of a corresponding reference subject. Further, the cell source fraction for the type of cancer in the reference biological sample in the corresponding reference subject is at least two percent, at least four percent, at least six percent, at least eight percent, at least ten percent, at least twelve percent, at least fourteen percent, at least sixteen percent, at least eighteen percent, at least twenty percent, at least thirty percent, at least forty percent, at least fifty percent, at least sixty percent, at least seventy percent, at least eighty percent or at least ninety percent.

In some embodiments, the second cell source is from one or more cells in a healthy cancer-free state.

In some embodiments, the first cell source or the second cell source is from a non-cancerous tissue. In some embodiments, the first cell source or the second cell source is from cells that derive from healthy tissue. In some embodiments, the first cell source or the second cell source is from a healthy tissue such as breast, lung, prostate, colorectal, renal, uterine, pancreatic, esophageal, lymph, ovarian, cervical, epidermal, thyroid, bladder, gastric, or a combination thereof.

In some embodiments the first cell source is any source identified in Example 8.

In some embodiments the second cell source is any source identified in Example 8.

In some embodiments, the method further comprises obtaining a methylation state of each nucleic acid fragment in a second plurality of nucleic acid fragments in electronic form from a second plurality of cell-free nucleic acid molecules in a second biological sample of the test subject at a second time period. In some embodiments, the method continues by individually assigning a second score to each respective nucleic acid fragment in the second plurality of nucleic acid fragments, thereby obtaining a plurality of second scores. In some embodiments, each respective second score represents a likelihood that the nucleic acid fragment was obtained from a cell-free nucleic acid molecule that originated from a circulating nucleic acid sample associated with the first cell source. In some embodiments, the individually assigning comprises: i) comparing the methylation state of the respective nucleic acid fragment against the first canonical set of methylation state vectors and against the second canonical set of methylation state vectors, or ii) presenting the methylation state of the respective nucleic acid fragment to the first classifier. In some embodiments, the method proceeds with transforming the plurality of second scores into a second plurality of counts. In some embodiments, each count in the second plurality of counts is for a methylation site in the first predetermined set of methylation sites in the genome of a reference sequence of the species. In some embodiments, the method continues by estimating a second instance of the first cell source fraction, in the test subject using the second plurality of counts by comparing the respective count of each respective methylation site in the first predetermined set of methylation sites represented by the second plurality of counts to a corresponding reference score for the respective methylation site in the first reference set. In some embodiments, the second time period is between a month and a year after the first time period. In some embodiments, the second time period is between a day and a month after the first time period.

In some embodiments, the method further comprises using a difference between the first instance of the first cell source fraction and the second instance of the first cell source fraction as a basis or a partial basis for determining an aggressiveness of the first cell source in the test subject.

In some embodiments, the method further comprises using a difference in the first instance of the first cell source fraction and the second instance of the first cell source fraction as a basis or a partial basis for determining a treatment option for a disease condition associated with the first cell source in the test subject.

In some embodiments, the first cell source is a type of cancer and the method further comprises using the first instance of the first cell source fraction as a basis or a partial basis for determining a stage of the type of cancer in the test subject.

In some embodiments, the first cell source is lymphocytes and the method further comprises using the first cell source fraction as a basis or a partial basis for evaluating a cancer condition of the test subject.

In some embodiments, the first cell source is a type of cancer and the method further comprises using the first cell source fraction as a basis or a partial basis for determining a treatment option for the first cell source in the test subject.

In some embodiments, the first canonical set of methylation state vectors is a single consensus methylation state vector of the genome of the species formed from a methylation state of nucleic acids in the tissue sample or cell-free nucleic acid sample across the first plurality of reference subjects. In some embodiments, the second canonical set of methylation state vectors is a single consensus methylation state vector of the genome of the species formed from a methylation state of nucleic acids in the tissue sample or cell-free nucleic acid sample across the second plurality of reference subjects.

In some embodiments, the first canonical set of methylation state vectors includes a different consensus methylation state vector of the genome of the species for each respective reference subject in the first plurality of reference subjects formed from a methylation state of nucleic acids in the tissue sample or cell-free nucleic acid sample of the respective reference subject. In some embodiments, the second canonical set of methylation state vectors includes a different consensus methylation state vector of the genome of the species for each respective reference subject in the second plurality of reference subjects formed from a methylation state of nucleic acids in the tissue sample or cell-free nucleic acid sample of the respective reference subject.

In some embodiments, the first plurality of reference subjects comprises at least ten reference subjects, and the second plurality of reference subjects comprises at least ten reference subjects.

In some embodiments, the first plurality of reference subjects comprises at least one hundred reference subjects, and the second plurality of reference subjects comprises at least one hundred reference subjects. In some embodiments, the first plurality of reference subjects includes more or less reference subjects than the second plurality of reference subjects.

In some embodiments, the first classifier is based on a multinomial logistic regression algorithm. In alternative embodiments, the first classifier is based on a neural network algorithm, a support vector machine algorithm, a Naive Bayes algorithm, a nearest neighbor algorithm, a boosted trees algorithm, a random forest algorithm, a convolutional neural network, a decision tree algorithm a mixture model, or a hidden Markov model.

As discussed above, in some embodiments, the individually assigning further assigns a second score to each respective nucleic acid fragment in the first plurality of nucleic acid fragments, thereby obtaining a plurality of second scores. Each respective second score in the plurality of second scores is for a nucleic acid fragment in the first plurality of nucleic acid fragments. Each respective second score represents a likelihood that the corresponding nucleic acid fragment was obtained from a cell-free nucleic acid molecule that originated from a third cell source. In such embodiments, the individually assigning described above further comprises i) comparing a methylation state of the respective nucleic acid fragment against at least a third canonical set of methylation state vectors and against the second canonical set of methylation state vectors, or ii) presenting the methylation state of the respective nucleic acid fragment to a second classifier trained at least in part on the third canonical set of methylation state vectors and the second canonical set of methylation state vectors. In such embodiments, each canonical methylation state vector in the third canonical set of methylation state vectors is derived from a respective third tissue sample or a respective third cell-free nucleic acid sample of a corresponding reference subject in a third plurality of reference subjects, where the respective third tissue sample or the respective third cell-free nucleic acid sample corresponds to the third cell source. In some embodiments, the transforming described above further comprises transforming the second plurality of scores into a second plurality of counts. Each count in the second plurality of counts is for a methylation site in a second predetermined set of methylation sites in the genome of a reference sequence of the species. Moreover, the second predetermined set of methylation sites is associated with the third cell source. In some such embodiments, the method proceeds by estimating a second cell source fraction in the first biological sample using the second plurality of counts by comparing the respective count of each respective methylation site in the second predetermined set of methylation sites represented by the second plurality of counts to a corresponding reference score for the respective methylation site in a second reference set. In such embodiments, each corresponding reference score in the second reference set is obtained by determining a frequency of methylation of the corresponding methylation site in nucleic acid fragments obtained from the respective third tissue sample or the respective third cell-free nucleic acid sample of a corresponding reference subject in the third plurality of reference subjects. In some embodiments, the individually assigning methodology described above provides the methylation state of the respective nucleic acid fragment against the second classifier. Further, in some such embodiments, the first classifier and the second classifier are the same. Further still, the first classifier is trained at least in part on the first canonical set of methylation state vectors, the second canonical set of methylation state vectors, and the third canonical set of methylation state vectors.

In some embodiments, the first classifier is other than the second classifier and the first classifier is not trained on the third canonical set of methylation state vectors.

In some embodiments, the first predetermined set of methylation sites comprises fifty methylation sites in the genome of the species, one hundred methylation sites in the genome of the species, or five hundred methylation sites in the genome of the species.

In some embodiments, the transforming the plurality of first scores into a first plurality of counts comprises, for each respective methylation site in the first predetermined set of methylation sites (a) determining a first number of nucleic acid fragments in the first plurality of nucleic acid fragments that (i) map to the respective methylation site and (ii) have a first score satisfying a threshold value, (b) determining a second number of nucleic acid fragments in the plurality of nucleic acid fragments that (i) map to the respective methylation site and (ii) have a first score satisfying or not satisfying the threshold value, and (c) assigning the respective methylation site as a quotient of the first number and the second number. In some such embodiments, the first score is a likelihood and the threshold value is fifty percent. In some such embodiments, a count of each respective nucleic acid fragment in the first number of nucleic acid fragments is down-weighted by its corresponding first score.

In some embodiments, each count of each respective methylation site in the first predetermined set of methylation sites is an observed frequency of methylation of the corresponding methylation site across the first plurality of nucleic acid fragments. In some such embodiments, the estimating further comprises constructing a Poisson model or a negative binomial distribution assumption using the count of each respective methylation site and the corresponding reference frequency each respective methylation site in the first reference set. Further, the Poisson model or the negative binomial distribution assumption is used to form a cumulative density function across a range of calculated first cell source fractions. In some embodiments, the method includes deeming the first instance of the first cell source fraction to be a mean of the cumulative density function across the range of calculated first cell source fractions.

In some embodiments, each count of each respective methylation site in the first predetermined set of methylation sites is an observed frequency of methylation of the corresponding methylation site across the first plurality of nucleic acid fragments. In some embodiments, the estimating further comprises constructing a respective Poisson model or a respective negative binomial distribution assumption using the count for each respective methylation site and the corresponding reference frequency of the methylation site in the first reference set, thereby constructing a plurality of Poisson models or a plurality of negative binomial distribution assumptions. In some such embodiments, the estimating further comprises using each respective Poisson model or each respective negative binomial distribution assumption to form a corresponding cumulative density function across a range of calculated first cell source fractions. In some embodiments, the estimating further comprises deeming the first instance of the first cell source fraction to be a combination of the mean of the cumulative density function across the range of calculated first cell source fractions combined across the plurality of Poisson models or the plurality of negative binomial distribution assumptions. In some embodiments, the range of calculated first cell source fractions is between zero and 110 percent.

In some embodiments, the first biological sample comprises blood, whole blood, plasma, serum, urine, cerebrospinal fluid, fecal, saliva, sweat, tears, pleural fluid, pericardial fluid, or peritoneal fluid of the test subject.

In some embodiments, the first biological sample consists of blood, whole blood, plasma, serum, urine, cerebrospinal fluid, fecal, saliva, sweat, tears, pleural fluid, pericardial fluid, or peritoneal fluid of the test subject.

In some embodiments, the first cell source is from one or more cells of a first cancer of a common primary site of origin. In some such embodiments, the first cancer is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof. Alternatively, in some such embodiments, the first cancer is a stage of a breast cancer, a stage of a lung cancer, a stage of a prostate cancer, a stage of a colorectal cancer, a stage of a renal cancer, a stage of a uterine cancer, a stage of a pancreatic cancer, a stage of a cancer of the esophagus, a stage of a lymphoma, a stage of a head/neck cancer, a stage of a ovarian cancer, a stage of a hepatobiliary cancer, a stage of a melanoma, a stage of a cervical cancer, a stage of a multiple myeloma, a stage of a leukemia, a stage of a thyroid cancer, a stage of a bladder cancer, or a stage of a gastric cancer.

Another aspect provides a computing system comprising one or more processors, and memory storing one or more programs to be executed by the one or more processor. The one or more programs comprise instructions for estimating a first cell source fraction in a first biological sample in a test subject of a given species by a method that comprises obtaining a methylation state of each nucleic acid fragment in a first plurality of nucleic acid fragments in electronic form from a first plurality of cell-free nucleic acid molecules in the first biological sample at a first time period. In the method, a first score is individually assigned to each respective nucleic acid fragment in the first plurality of nucleic acid fragments, thereby obtaining a plurality of first scores. Each respective first score represents a likelihood that the corresponding nucleic acid fragment was obtained from a cell-free nucleic acid molecule that originated from the first cell source. The individually assigning comprises i) comparing a methylation state of the respective nucleic acid fragment against a first canonical set of methylation state vectors and against a second canonical set of methylation state vectors representative of a source other than the first cell source, or ii) presenting the methylation state of the respective nucleic acid fragment to a first classifier trained at least in part on the first canonical set of methylation state vectors and the second canonical set of methylation state vectors. Each canonical methylation state vector in the first canonical set of methylation state vectors is derived from a tissue sample or cell-free nucleic acid sample of a corresponding reference subject in a first plurality of reference subjects corresponding to the first cell source. Each canonical methylation state vector in the second canonical set of methylation state vectors is derived from a tissue sample or cell-free nucleic acid sample of a corresponding reference subject in a second plurality of reference subjects corresponding to a second cell source. The method continues by transforming the plurality of first scores into a first plurality of counts. Each count in the first plurality of counts is for a methylation site in a first predetermined set of methylation sites in the genome of a reference sequence of the species. The first predetermined set of methylation sites is associated with the first cell source. The method continues by estimating a first instance of the first cell source fraction in the first biological sample using the first plurality of counts by comparing the respective count of each respective methylation site in the first predetermined set of methylation sites represented by the first plurality of counts to a corresponding reference score for the respective methylation site in a first reference set. Each corresponding reference score in the first reference set is obtained by determining a frequency of methylation of the corresponding methylation site in nucleic acid fragments obtained from the tissue sample or cell-free nucleic acid sample of a corresponding reference subject in the first plurality of reference subjects. In another aspect, the one or more programs further comprise instructions for performing any of the methods disclosed above alone or in combination.

Still another aspect of the present disclosure provides non-transitory computer readable storage medium storing one or more programs for estimating a first cell source fraction in a first biological sample in a test subject of a given species. The one or more programs are configured for execution by a computer. Moreover, the one or more programs comprise instructions for obtaining a methylation state of each nucleic acid fragment in a first plurality of nucleic acid fragments in electronic form from a first plurality of cell-free nucleic acid molecules in the first biological sample at a first time period. The one or more programs further comprises instructions for individually assigning a first score to each respective nucleic acid fragment in the first plurality of nucleic acid fragments, thereby obtaining a plurality of first scores. Each respective first score represents a likelihood that the corresponding nucleic acid fragment was obtained from a cell-free nucleic acid molecule that originated from the first cell source. The individually assigning (B) comprises i) comparing a methylation state of the respective nucleic acid fragment against a first canonical set of methylation state vectors and against a second canonical set of methylation state vectors representative of a source other than the first cell source, or ii) presenting the methylation state of the respective nucleic acid fragment to a first classifier trained at least in part on the first canonical set of methylation state vectors and the second canonical set of methylation state vectors. Each canonical methylation state vector in the first canonical set of methylation state vectors is derived from a tissue sample or cell-free nucleic acid sample of a corresponding reference subject in a first plurality of reference subjects corresponding to the first cell source. Each canonical methylation state vector in the second canonical set of methylation state vectors is derived from a tissue sample or cell-free nucleic acid sample of a corresponding reference subject in a second plurality of reference subjects corresponding to a second cell source. The one or more programs further comprises instructions for transforming the plurality of first scores into a first plurality of counts. Each count in the first plurality of counts is for a methylation site in a first predetermined set of methylation sites in the genome of a reference sequence of the species. The first predetermined set of methylation sites is associated with the first cell source. The one or more programs further comprises instructions for estimating a first instance of the first cell source fraction in the first biological sample using the first plurality of counts by comparing the respective count of each respective methylation site in the first predetermined set of methylation sites represented by the first plurality of counts to a corresponding reference score for the respective methylation site in a first reference set, wherein each corresponding reference score in the first reference set is obtained by determining a frequency of methylation of the corresponding methylation site in nucleic acid fragments obtained from the tissue sample or cell-free nucleic acid sample of a corresponding reference subject in the first plurality of reference subjects. Still another aspect of the present disclosure provides non-transitory computer readable storage medium in which the one or more programs further comprise instructions for performing any of the methods disclosed above alone or in combination.

B. Embodiments that estimate the cell source fraction for each of a plurality of cell sources by making use of the transformation of nucleic acid fragment scores to methylation counts. Another aspect of the present disclosure provides a method of estimating a respective cell source fraction in a first biological sample in a test subject of a given species for each cell source in a plurality of cell sources thereby estimating a plurality of cell source fractions. In some such embodiments, the plurality of cell sources comprises two different cell sources, three different cell sources, four different cell sources, five different cell sources, or more than five different cell sources. In accordance with this aspect of the present disclosure, a method is provided that comprises, at a computer system having one or more processors, and memory storing one or more programs for execution by the one or more processors, obtaining a methylation state of each nucleic acid fragment in a first plurality of nucleic acid fragments in electronic form from a first plurality of cell-free nucleic acid molecules in a first biological sample of a test subject at a first time period. In the method, a plurality of scores is individually assigned to each respective nucleic acid fragment in the first plurality of nucleic acid fragments, thereby obtaining a first plurality of score sets where each score set comprises a plurality of scores corresponding to the number of reference cell sources available. Each respective score set in the plurality of score sets is for a corresponding nucleic acid fragment in the first plurality of nucleic acid fragments. Each respective score in each respective score set in the plurality of score sets represents a likelihood that the corresponding nucleic acid fragment was obtained from a cell-free nucleic acid molecule that originated from the corresponding different cell source in the plurality of cell sources. The individually assigning comprises i) comparing a methylation state of the respective nucleic acid fragment against a plurality of canonical sets of methylation state vectors, or ii) presenting the methylation state of the respective nucleic acid fragment to a classifier trained at least in part on the plurality of canonical sets of methylation state vectors, each corresponding to a cell source. Each canonical methylation state vector in each canonical set of methylation state vectors in the plurality of canonical sets of methylation state vectors is derived from a tissue sample or cell-free nucleic acid sample of a corresponding reference subject in a plurality of reference subjects. The plurality of reference subjects includes at least one representative reference subject for each respective cell source in the plurality of cell sources. In the method, each score set, in the plurality of scores sets, is transformed into a plurality of count sets. Each respective count set in the plurality of count sets represents a different cell source in the plurality of cell sources. For each respective count set, each count in the respective count set is for a methylation site in a corresponding predetermined set of methylation sites in the genome of a reference sequence of the species that corresponds to the cell source represented by the respective count set. In the method, the plurality of cell source fractions in the test subject is estimated using the plurality of count sets. Such estimation comprises, for each respective count set in the plurality of count sets, comparing the respective count of each respective methylation site in the predetermined set of methylation sites in the respective count set to a corresponding reference score for the respective methylation site in a corresponding reference set. Each corresponding reference score in the corresponding reference set is obtained by determining a frequency of methylation of the corresponding methylation site in nucleic acid fragments obtained from a tissue sample or cell-free nucleic acid sample of a corresponding reference subject in the plurality of reference subjects corresponding to the cell source represented by the count set.

In some such embodiments, each canonical methylation state vector in a first canonical set of methylation state vectors in the plurality of canonical sets of methylation state vectors represents the methylation state across the genome of the corresponding reference subject in the first plurality of reference subjects.

In alternative embodiments, each canonical methylation state vector in a first canonical set of methylation state vectors in the plurality of canonical sets of methylation state vectors represents the methylation state across a subset of the genome of the corresponding reference subject. A methylation state of the subset of the genome is representative of causative biology underlying a first cell source in the plurality of cell sources.

In some embodiments, each cell source in the plurality of cell sources is a different cancer type in a plurality of cancer types, and a canonical methylation state vector in a first canonical set of methylation state in the plurality of canonical sets of methylation state vectors is derived from a sample of a tumor of a type of cancer in the plurality of cancer types obtained from the corresponding reference subject.

In some embodiments, each cell source in the plurality of cell sources is a different cancer type in a plurality of cancer types, and a canonical methylation state vector in a first set of canonical methylation state vectors in the plurality of canonical sets of methylation state vectors is derived from cell-free nucleic acids of a reference biological sample from a reference subject. In such embodiments, a tumor fraction in the reference biological sample, with respect to a first cancer type in the plurality of cancer types, for the corresponding reference subject is at least at least two percent, at least four percent, at least six percent, at least eight percent, at least ten percent, at least twelve percent, at least fourteen percent, at least sixteen percent, at least eighteen percent, at least twenty percent, at least thirty percent, at least forty percent, at least fifty percent, at least sixty percent, at least seventy percent, at least eighty percent or at least ninety percent.

In some embodiments, a first cell source in the plurality of cell sources is a type of cancer and a second cell source in the plurality of cell sources is cancer-free cells.

In some embodiments, a first cell source in the plurality of cell sources is a type of cancer and the method further comprises using an estimated cell source fraction for the first cell source in the plurality of cell source fractions as a basis or a partial basis for determining a stage of the type of cancer in the test subject.

In some embodiments, a first cell source in the plurality of cell sources is lymphocytes and the method further comprises using an estimated cell source fraction for the first cell source in the plurality of cell source fractions as a basis or a partial basis for evaluating a cancer condition of the test subject.

In some embodiments, a first cell source in the plurality of cell sources is a type of cancer and the method further comprises using an estimated cell source fraction for the first cell source in the plurality of cell source fractions as a basis or a partial basis for determining a treatment option for the type of cancer in the test subject.

In some embodiments, the individually assigning comprises presenting the methylation state of the respective nucleic acid fragment to the classifier trained at least in part on the plurality of canonical sets of methylation state vectors, and the classifier is based on a multinomial logistic regression algorithm.

In some embodiments, the individually assigning comprises presenting the methylation state of the respective nucleic acid fragment to the classifier trained at least in part on the plurality of canonical sets of methylation state vectors, and the classifier is based on a neural network algorithm, a support vector machine algorithm, a Naive Bayes algorithm, a nearest neighbor algorithm, a boosted trees algorithm, a random forest algorithm, a convolutional neural network, a decision tree algorithm a mixture model, or a hidden Markov model.

In some embodiments, a corresponding predetermined set of methylation sites comprises fifty methylation sites in the genome of the species, one hundred methylation sites in the genome of the species, or five hundred methylation sites in the genome of the species.

In some embodiments, the transforming the plurality of score sets into the plurality of count sets comprises, for each respective methylation site in a corresponding predetermined set of methylation sites (a) determining a first number of nucleic acid fragments in the first plurality of nucleic acid fragments that (i) map to the respective methylation site and (ii) have a first score satisfying a threshold value, (b) determining a second number of nucleic acid fragments in the plurality of nucleic acid fragments that (i) map to the respective methylation site and (ii) have a first score satisfying or not satisfying the threshold value, and (c) assigning the respective count for the methylation site as a quotient of the first number and the second number. In some such embodiments, the first score is a likelihood and the threshold value is 0.5. In some embodiments, a count of each respective nucleic acid fragment in the first number of nucleic acid fragments is down-weighted by its corresponding first score.

In some embodiments, the first biological sample comprises blood, whole blood, plasma, serum, urine, cerebrospinal fluid, fecal, saliva, sweat, tears, pleural fluid, pericardial fluid, or peritoneal fluid of the test subject.

In some embodiments, the first biological sample consists of blood, whole blood, plasma, serum, urine, cerebrospinal fluid, fecal, saliva, sweat, tears, pleural fluid, pericardial fluid, or peritoneal fluid of the test subject.

In some embodiments, a cell source in the plurality of cell sources is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof.

In some embodiments, a cell source in the plurality of cell sources is a stage of a breast cancer, a stage of a lung cancer, a stage of a prostate cancer, a stage of a colorectal cancer, a stage of a renal cancer, a stage of a uterine cancer, a stage of a pancreatic cancer, a stage of a cancer of the esophagus, a stage of a lymphoma, a stage of a head/neck cancer, a stage of an ovarian cancer, a stage of a hepatobiliary cancer, a stage of a melanoma, a stage of a cervical cancer, a stage of a multiple myeloma, a stage of a leukemia, a stage of a thyroid cancer, a stage of a bladder cancer, or a stage of a gastric cancer.

In some embodiments, the test subject is human and each reference subject is human.

In some embodiments a source in the plurality of cell source is any source identified in Example 8. In some embodiments each cell source in the plurality of cell source is any source identified in Example 8.

Another aspect of the present disclosure provides a computing system, comprising one or more processors, and memory storing one or more programs to be executed by the one or more processor. The one or more programs comprise instructions of estimating a respective cell source fraction in a first biological sample in a test subject of a given species for each cell source in a plurality of cell sources thereby estimating a plurality of cell source fractions by a method. The method comprises obtaining a methylation state of each nucleic acid fragment in a first plurality of nucleic acid fragments in electronic form from a first plurality of cell-free nucleic acid molecules in a first biological sample of the test subject at a first time period. The method further comprises individually assigning a plurality of scores to each respective nucleic acid fragment in the first plurality of nucleic acid fragments, thereby obtaining a first plurality of score sets where each score set comprises a plurality of scores corresponding to the number of reference cell sources available. Each respective score set in the plurality of score sets is for a corresponding nucleic acid fragment in the first plurality of nucleic acid fragments. Each respective score in each respective score set in the plurality of score sets represents a likelihood that the corresponding nucleic acid fragment was obtained from a cell-free nucleic acid molecule that originated from the corresponding different cell source in the plurality of cell sources. The individually assigning comprises i) comparing a methylation state of the respective nucleic acid fragment against a plurality of canonical sets of methylation state vectors, or ii) presenting the methylation state of the respective nucleic acid fragment to a classifier trained at least in part on the plurality of canonical sets of methylation state vectors, each corresponding to a cell source. Each canonical methylation state vector in each canonical set of methylation state vectors in the plurality of canonical sets of methylation state vectors is derived from a tissue sample or cell-free nucleic acid sample of a corresponding reference subject in a plurality of reference subjects. The plurality of reference subjects includes at least one representative reference subject for each respective cell source in the plurality of cell sources. The method further comprises transforming the plurality of scores sets into a plurality of count sets. Each respective count set in the plurality of count sets represents a different cell source in the plurality of cell sources, where, for each respective count set, each count in the respective count set is for a methylation site in a corresponding predetermined set of methylation sites in the genome of a reference sequence of the species that corresponds to the cell source represented by the respective count set. The method further comprises estimating the plurality of cell source fractions in the test subject using the plurality of count sets. This estimation comprises, for each respective count set in the plurality of count sets, comparing the respective count of each respective methylation site in the predetermined set of methylation sites in the respective count set to a corresponding reference score for the respective methylation site in a corresponding reference set. Each corresponding reference score in the corresponding reference set is obtained by determining a frequency of methylation of the corresponding methylation site in nucleic acid fragments obtained from a tissue sample or cell-free nucleic acid sample of a corresponding reference subject in the plurality of reference subjects corresponding to the cell source represented by the count set. Another aspect of the present disclosure provides a computing system including the above disclosed one or more programs that further comprise instructions for performing any of the above disclosed methods alone or in combination.

Another aspect of the present disclosure provides a non-transitory computer readable storage medium storing one or more programs for of estimating a respective cell source fraction in a first biological sample in a test subject of a given species for each cell source in a plurality of cell sources thereby estimating a plurality of cell source fractions. The one or more programs are configured for execution by a computer. Moreover, the one or more programs comprise instructions for obtaining a methylation state of each nucleic acid fragment in a first plurality of nucleic acid fragments in electronic form from a first plurality of cell-free nucleic acid molecules in a first biological sample of the test subject at a first time period. The one or more programs further comprise instructions for individually assigning a plurality of scores to each respective nucleic acid fragment in the first plurality of nucleic acid fragments, thereby obtaining a first plurality of score sets where each score set comprises a plurality of scores corresponding to the number of reference cell sources available. Each respective score set in the plurality of score sets is for a corresponding nucleic acid fragment in the first plurality of nucleic acid fragments. Each respective score in each respective score set in the plurality of score sets represents a likelihood that the corresponding nucleic acid fragment was obtained from a cell-free nucleic acid molecule that originated from the corresponding different cell source in the plurality of cell sources. The individually assigning comprises i) comparing a methylation state of the respective nucleic acid fragment against a plurality of canonical sets of methylation state vectors, or ii) presenting the methylation state of the respective nucleic acid fragment to a classifier trained at least in part on the plurality of canonical sets of methylation state vectors, each corresponding to a cell source. Each canonical methylation state vector in each canonical set of methylation state vectors in the plurality of canonical sets of methylation state vectors is derived from a tissue sample or cell-free nucleic acid sample of a corresponding reference subject in a plurality of reference subjects. The plurality of reference subjects includes at least one representative reference subject for each respective cell source in the plurality of cell sources. The one or more programs further comprise instructions for transforming the plurality of scores sets into a plurality of count sets. Each respective count set in the plurality of count sets represents a different cell source in the plurality of cell sources. For each respective count set, each count in the respective count set is for a methylation site in a corresponding predetermined set of methylation sites in the genome of a reference sequence of the species that corresponds to the cell source represented by the respective count set. The one or more programs further comprise instructions for estimating the plurality of cell source fractions in the test subject using the plurality of count sets. The estimating (D) comprises, for each respective count set in the plurality of count sets, comparing the respective count of each respective methylation site in the predetermined set of methylation sites in the respective count set to a corresponding reference score for the respective methylation site in a corresponding reference set. Each corresponding reference score in the corresponding reference set is obtained by determining a frequency of methylation of the corresponding methylation site in nucleic acid fragments obtained from a tissue sample or cell-free nucleic acid sample of a corresponding reference subject in the plurality of reference subjects corresponding to the cell source represented by the count set.

In some embodiments, a cell source is from a non-cancerous tissue. In some embodiments, a cell source is from cells that derive from healthy tissue. In some embodiments, a cell source is from a healthy tissue such as breast, lung, prostate, colorectal, renal, uterine, pancreatic, esophageal, lymph, ovarian, cervical, epidermal, thyroid, bladder, gastric, or a combination thereof.

Another aspect of the present disclosure provides non-transitory computer readable storage medium comprising the above-disclosed one or more programs in which the one or more programs further comprise instructions for performing any of the above-disclosed methods alone or in combination.

C. Embodiments that train a classifier to discriminate between a first cell source and a second cell source. Another aspect of the present disclosure provides a classification method comprising, at a computer system having one or more processors, and memory storing one or more programs for execution by the one or more processors, for each respective reference subject in a first plurality of reference subjects, where each reference subject in the first plurality of reference subjects has a first cell source, obtaining a methylation state of each nucleic acid fragment in a first plurality of nucleic acid fragments in electronic form from a biological sample of the respective reference subject. The one or more programs use the methylation state of each nucleic acid fragment in the first plurality of nucleic acid fragments to generate a corresponding methylation state vector, thereby obtaining a first canonical set of methylation state vectors. The one or more programs, for each respective reference subject in a second plurality of reference subjects, where each reference subject in the second plurality of reference subjects has a second cell source, obtain a methylation state of each nucleic acid fragment in a second plurality of nucleic acid fragments in electronic form from a biological sample of the respective reference subject. The one or more programs use the methylation state of each nucleic acid fragment in the second plurality of nucleic acid fragments to generate a corresponding methylation state vector, thereby obtaining a second canonical set of methylation state vectors. The one or more programs apply the first and second canonical sets of methylation state vectors collectively to an untrained or partially trained classifier, in conjunction with a cell source of each respective reference subject in the first plurality of reference subjects and the second plurality of reference subjects, thereby obtaining a trained classifier that discriminates between the first cell source and the second cell source.

In some such embodiments, the first cell source is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, or gastric cancer.

In some embodiments, the second cell source is healthy cancer-free cells.

In some embodiments, the first cell source or the second cell source is from a non-cancerous tissue. In some embodiments, the first cell source or the second cell source is from cells that derive from healthy tissue. In some embodiments, the first cell source or the second cell source is from a healthy tissue such as breast, lung, prostate, colorectal, renal, uterine, pancreatic, esophageal, lymph, ovarian, cervical, epidermal, thyroid, bladder, gastric, or a combination thereof.

In some embodiments the first cell source is any cell source identified in Example 8. In some embodiments the second cell source is any cell source identified in Example 8.

In some alternative embodiments, the second cell source is other than the first cell source, and the second cell source is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, or gastric cancer.

In some embodiments, each first plurality of nucleic acid fragments is derived from a tissue sample or cell-free nucleic acid sample of a corresponding first reference subject.

In some embodiments, each second plurality of nucleic acid fragments is derived from a tissue sample or cell-free nucleic acid sample of a corresponding second reference subject.

In some embodiments, the untrained or partially trained classifier is based on a neural network algorithm, a support vector machine algorithm, a decision tree algorithm, an unsupervised clustering algorithm, a supervised clustering algorithm, a logistic regression algorithm, a mixture model, or a hidden Markov model. In some embodiments, the untrained or partially trained classifier is a multinomial classifier.

In some embodiments, the method further comprises obtaining a methylation state of each nucleic acid fragment in a plurality of test nucleic acid fragments in electronic form from a plurality of cell-free nucleic acid molecules in a test biological sample from a test subject that is not in the first plurality of reference subjects or the second plurality of reference subjects. In such embodiments, the method further comprises individually assigning a first score to each respective nucleic acid fragment in the plurality of test nucleic acid fragments, thereby obtaining a plurality of first scores. Each respective first score represents a likelihood that the corresponding nucleic acid fragment was obtained from a cell-free nucleic acid molecule that originated from the first cell source. The individually assigning comprises presenting the methylation state of the respective test nucleic acid fragment to the trained classifier. The method further comprises transforming the plurality of first scores into a first plurality of counts. Each count in the first plurality of counts is for a methylation site in a first predetermined set of methylation sites in the genome of a reference sequence of the species. The first predetermined set of methylation sites is associated with the first cell source. The method further comprises estimating a first cell source fraction in the first biological sample using the first plurality of counts by comparing the respective count of each respective methylation site in the first predetermined set of methylation sites represented by the first plurality of counts to a corresponding reference score for the respective methylation site in the first reference set.

Another aspect of the present disclosure provides a computing system. The computing system comprises one or more processors and memory storing one or more programs to be executed by the one or more processor. The one or more programs comprises instructions for classification by a method. In the method, for each respective reference subject in a first plurality of reference subjects, where each reference subject in the first plurality of reference subjects has a first cell source, a methylation state of each nucleic acid fragment in a first plurality of nucleic acid fragments is obtained in electronic form from a biological sample of the respective reference subject. The methylation state of each nucleic acid fragment in the first plurality of nucleic acid fragments is used to generate a corresponding methylation state vector, thereby obtaining a first canonical set of methylation state vectors. In the method, for each respective reference subject in a second plurality of reference subjects, where each reference subject in the second plurality of reference subjects has a second cell source, a methylation state of each nucleic acid fragment in a second plurality of nucleic acid fragments is obtained in electronic form from a biological sample of the respective reference subject. The methylation state of each nucleic acid fragment in the second plurality of nucleic acid fragments is used to generate a corresponding methylation state vector, thereby obtaining a second canonical set of methylation state vectors. In the method, the first and second canonical sets of methylation state vectors are collectively applied to an untrained or partially trained classifier, in conjunction with a cell source of each respective reference subject in the first plurality of reference subjects and the second plurality of reference subjects, thereby obtaining a trained classifier that discriminates between the first cell source and the second cell source. Another aspect of the present disclosure provides the above-disclosed computing system where the one or more programs further comprise instructions for performing any of the methods disclosed above alone or in combination.

Another aspect of the present disclosure provides a non-transitory computer readable storage medium storing one or more programs for classification. The one or more programs are configured for execution by a computer. The one or more programs comprise instructions that, for each respective reference subject in a first plurality of reference subjects, where each reference subject in the first plurality of reference subjects has a first cell source, obtaining a methylation state of each nucleic acid fragment in a first plurality of nucleic acid fragments in electronic form from a biological sample of the respective reference subject. The one or more programs comprise instructions for using the methylation state of each nucleic acid fragment in the first plurality of nucleic acid fragments to generate a corresponding methylation state vector, thereby obtaining a first canonical set of methylation state vectors. The one or more programs further comprise instructions that, for each respective reference subject in a second plurality of reference subjects, where each reference subject in the second plurality of reference subjects has a second cell source, obtain a methylation state of each nucleic acid fragment in a second plurality of nucleic acid fragments in electronic form from a biological sample of the respective reference subject. The one or more programs further comprise instructions that use the methylation state of each nucleic acid fragment in the second plurality of nucleic acid fragments to generate a corresponding methylation state vector, thereby obtaining a second canonical set of methylation state vectors. The one or more programs comprise instructions for applying the first and second canonical sets of methylation state vectors collectively to an untrained or partially trained classifier, in conjunction with a cell source of each respective reference subject in the first plurality of reference subjects and the second plurality of reference subjects, thereby obtaining a trained classifier that discriminates between the first cell source and the second cell source. Another aspect of the present disclosure provides the above-disclosed non-transitory computer readable storage medium in which the one or more programs further comprise instructions for performing any of the above-disclosed methods alone or in combination.

D. Embodiments that estimate the cell source fraction for at least one cell source without making use of a transformation of nucleic acid fragment scores to methylation counts. The above disclosed methods are useful particularly in instances when the cell source fraction is below levels such as one in ten thousand, one in five thousand or one in five hundred. In instances where the cell source fraction is higher, such as 1 in one hundred, or five in one hundred, more coarse-grained methods can be used to estimate cell source fraction. In such methods, nucleic acid fragments are scored for cell source origin and such scores are directly used to ascertain cell source fraction without transforming such nucleic acid fragments into sets of methylation scores. In accordance with one such embodiment, a method of estimating a first cell source fraction in a first biological sample in a test subject of a given species is provides in which, at a computer system having one or more processors, and memory storing one or more programs for execution by the one or more processors, a methylation state of each nucleic acid fragment in a first plurality of nucleic acid fragments is obtained in electronic form from a first plurality of cell-free nucleic acid molecules in the first biological sample at a first time period. In the method, a first score is individually assigned to each respective nucleic acid fragment in the first plurality of nucleic acid fragments, thereby obtaining a plurality of first scores. Each respective first score represents a likelihood that the corresponding nucleic acid fragment was obtained from a cell-free nucleic acid molecule that originated from the first cell source. The individually assigning comprises i) comparing a methylation state of the respective nucleic acid fragment against a first canonical set of methylation state vectors and against a second canonical set of methylation state vectors representative of a source other than the first cell source, or ii) presenting the methylation state of the respective nucleic acid fragment to a first classifier trained at least in part on the first canonical set of methylation state vectors and the second canonical set of methylation state vectors. Each canonical methylation state vector in the first canonical set of methylation state vectors is derived from a tissue sample or cell-free nucleic acid sample of a corresponding reference subject in a first plurality of reference subjects corresponding to the first cell source. Each canonical methylation state vector in the second canonical set of methylation state vectors is derived from a tissue sample or cell-free nucleic acid sample of a corresponding reference subject in a second plurality of reference subjects corresponding to a second cell source. In the method, a first instance of the first cell source fraction in the first biological sample is estimated using the first score of each respective nucleic acid fragment in the first plurality of nucleic acid fragments by evaluating (i) a number of nucleic acid fragments that have a first score that satisfies a first predetermined threshold against (ii) the total number of nucleic acid fragments in the first plurality of nucleic acid fragments.

In some such embodiments, each canonical methylation state vector in the first canonical set of methylation state vectors represents the methylation state across the genome of the corresponding reference subject in the first plurality of reference subjects.

In some embodiments, each canonical methylation state vector in the first canonical set of methylation state vectors represents the methylation state across a subset of the genome of the corresponding reference subject. In such embodiments, a methylation state of the subset of the genome is representative of causative biology underlying the first cell source.

In some embodiments, the first cell source is a type of cancer, and a canonical methylation state vector in the first canonical set of methylation state vectors is derived from a sample of a tumor of the type of cancer obtained from the corresponding reference subject.

In some embodiments, the first cell source is a type of cancer, a canonical methylation state vector in the first set of canonical methylation state vectors is derived from cell-free nucleic acids of a reference biological sample from the corresponding reference subject, and the tumor fraction in the reference biological sample, with respect to the first cell source, for the corresponding reference subject is at least at least two percent, at least four percent, at least six percent, at least eight percent, at least ten percent, at least twelve percent, at least fourteen percent, at least sixteen percent, at least eighteen percent, at least twenty percent, at least thirty percent, at least forty percent, at least fifty percent, at least sixty percent, at least seventy percent, at least eighty percent or at least ninety percent.

In some embodiments, the second cell source is one or more cell types that are cancer-free. In some embodiments the first cell source is any source identified in Example 8. In some embodiments the second cell source is any source identified in Example 8.

In some embodiments, the first cell source or the second cell source is from a non-cancerous tissue. In some embodiments, the first cell source or the second cell source is from cells that derive from healthy tissue. In some embodiments, the first cell source or the second cell source is from a healthy tissue such as breast, lung, prostate, colorectal, renal, uterine, pancreatic, esophageal, lymph, ovarian, cervical, epidermal, thyroid, bladder, gastric, or a combination thereof.

In some embodiments, the method further comprises obtaining a methylation state of each nucleic acid fragment in a second plurality of nucleic acid fragments in electronic form from a second plurality of cell-free nucleic acid molecules in a second biological sample of the test subject at a second time period. The method further comprises individually assigning a second score to each respective nucleic acid fragment in the second plurality of nucleic acid fragments, thereby obtaining a plurality of second scores. Each respective second score represents a likelihood that the nucleic acid fragment was obtained from a cell-free nucleic acid molecule that originated from the first cell source. The individually assigning comprises: i) comparing the methylation state of the respective nucleic acid fragment against the first canonical set of methylation state vectors and against the second canonical set of methylation state vectors, or ii) presenting the methylation state of the respective nucleic acid fragment to the first classifier. The method further comprises estimating a second instance of the first cell source fraction in the second biological sample using the second score of each respective nucleic acid fragment in the second plurality of nucleic acid fragments by evaluating (i) a number nucleic acid fragments that have the second score that satisfies a predetermined threshold against (ii) the total number of nucleic acid fragments in the second plurality of nucleic acid fragments.

In some embodiments, the second time period is between a month and a year after the first time period. In some embodiments, the second time period is between a day and a month after the first time period.

In some embodiments, the method further comprises using a difference between the first instance of the first cell source fraction and the second instance of the first cell source fraction as a basis or a partial basis for determining an aggressiveness of a disease condition associated with the first cell source in the test subject.

In some embodiments, the method further comprises using a difference between the first instance of the first cell source fraction and the second instance of the first cell source fraction as a basis or a partial basis for determining a treatment option for a disease condition associated with the first cell source in the test subject.

In some embodiments, the first cell source is a type of cancer and the method further comprises using the first instance of the first cell source fraction as a basis or a partial basis for determining a stage of the type of cancer in the test subject.

In some embodiments, the first cell source is lymphocytes and the method further comprises using the first instance of the first cell source fraction as a basis or a partial basis for evaluating a cancer condition of the test subject.

In some embodiments, the first cell source is a type of cancer and the method further comprises using the first cell source fraction as a basis or a partial basis for determining a treatment option for the cancer in the test subject.

In some embodiments, the first canonical set of methylation state vectors is a single consensus methylation state vector of the genome of the species formed from a methylation state of nucleic acids in the tissue sample or cell-free nucleic acid sample across the first plurality of reference subjects, and the second canonical set of methylation state vectors is a single consensus methylation state vector of the genome of the species formed from a methylation state of nucleic acids in the tissue sample or cell-free nucleic acid sample across the second plurality of reference subjects.

In some embodiments, the first canonical set of methylation state vectors includes a different consensus methylation state vector of the genome of the species for each respective reference subject in the first plurality of reference subjects formed from a methylation state of nucleic acids in the tissue sample or cell-free nucleic acid sample of the respective reference subject, and the second canonical set of methylation state vectors includes a different consensus methylation state vector of the genome of the species for each respective reference subject in the second plurality of reference subjects formed from a methylation state of nucleic acids in the tissue sample or cell-free nucleic acid sample of the respective reference subject.

In some embodiments, the first plurality of reference subjects comprises at least ten reference subjects, and the second plurality of reference subjects comprises at least ten reference subjects other than the first plurality of reference subjects. In some embodiments, the first plurality of reference subjects comprises at least one hundred reference subjects, and the second plurality of reference subjects comprises at least one hundred reference subjects other than the first plurality of reference subjects. In some embodiments, the first plurality of reference subjects includes more or less reference subjects than the second plurality of reference subjects.

In some embodiments, the individually assigning comprises presenting the methylation state of the respective nucleic acid fragment to the first classifier, and the first classifier is based on a multinomial logistic regression algorithm. In some embodiments, the individually assigning comprises presenting the methylation state of the respective nucleic acid fragment to the first classifier, and the first classifier is based on a neural network algorithm, a support vector machine algorithm, a Naive Bayes algorithm, a nearest neighbor algorithm, a boosted trees algorithm, a random forest algorithm, a convolutional neural network, a decision tree algorithm a mixture model, or a hidden Markov model.

In some embodiments, the individually assigning further assigns a second score to each respective nucleic acid fragment in the first plurality of nucleic acid fragments, thereby obtaining a plurality of second scores, each respective second score in the plurality of second scores for a nucleic acid fragment in the first plurality of nucleic acid fragments, where each respective second score represents a likelihood that the corresponding nucleic acid fragment was obtained from a cell-free nucleic acid molecule that originated from a third cell source, the individually assigning further comprises i) comparing a methylation state of the respective nucleic acid fragment against at least a third canonical set of methylation state vectors and against the second canonical set of methylation state vectors, or ii) presenting the methylation state of the respective nucleic acid fragment to a second classifier trained at least in part on the third canonical set of methylation state vectors and the second canonical set of methylation state vectors, each canonical methylation state vector in the third canonical set of methylation state vectors is derived from a tissue sample or cell-free nucleic acid sample of a corresponding reference subject in a third plurality of reference subjects corresponding to the third cell source, and the estimating further comprises estimating a second cell source fraction in the first biological sample using the second score of each respective nucleic acid fragment in the first plurality of nucleic acid fragments by evaluating (i) a number of nucleic acid fragments that have a second score that satisfies a second predetermined threshold against (ii) the total number of nucleic acid fragments in the first plurality of nucleic acid fragments.

In some such embodiments, the individually assigning provides the methylation state of the respective nucleic acid fragment against the second classifier, the first classifier and the second classifier are the same, and the first classifier is trained at least in part on the first canonical set of methylation state vectors, the second canonical set of methylation state vectors, and the third canonical set of methylation state vectors.

In some embodiments, wherein the first classifier is other than the second classifier and the first classifier is not trained on the third canonical set of methylation state vectors.

In some embodiments, the first biological sample comprises blood, whole blood, plasma, serum, urine, cerebrospinal fluid, fecal, saliva, sweat, tears, pleural fluid, pericardial fluid, or peritoneal fluid of the test subject. In some embodiments, the first biological sample consists of blood, whole blood, plasma, serum, urine, cerebrospinal fluid, fecal, saliva, sweat, tears, pleural fluid, pericardial fluid, or peritoneal fluid of the test subject.

In some embodiments, the first cell source is one or more cells of a first cancer of a common primary site of origin. In some such embodiments the first cancer is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof. Alternatively, in some such embodiments, the first cancer is a stage of a breast cancer, a stage of a lung cancer, a stage of a prostate cancer, a stage of a colorectal cancer, a stage of a renal cancer, a stage of a uterine cancer, a stage of a pancreatic cancer, a stage of a cancer of the esophagus, a stage of a lymphoma, a stage of a head/neck cancer, a stage of a ovarian cancer, a stage of a hepatobiliary cancer, a stage of a melanoma, a stage of a cervical cancer, a stage of a multiple myeloma, a stage of a leukemia, a stage of a thyroid cancer, a stage of a bladder cancer, or a stage of a gastric cancer.

In some embodiments the test subject is human and each reference subject in the first plurality and second plurality of reference subjects is human.

Another aspect of the present disclosure provides a computing system comprising one or more processors and memory storing one or more programs to be executed by the one or more processors, the one or more programs comprising instructions for estimating a first cell source fraction in a first biological sample in a test subject of a given species by any of the methods disclosed above.

Still another aspect of the present disclosure provides a non-transitory computer readable storage medium storing one or more programs for estimating a first cell source fraction in a first biological sample in a test subject of a given species. The one or more programs are configured for execution by a computer. The one or more programs comprise instructions for performing any of the methods disclosed above.

Various embodiments of systems, methods and devices within the scope of the appended claims each have several aspects, no single one of which is solely responsible for the desirable attributes described herein. Without limiting the scope of the appended claims, some prominent features are described herein. After considering this discussion, and particularly after reading the section entitled “Detailed Description” one will understand how the features of various embodiments are used.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIGS. 1A and 1B illustrate an example block diagram illustrating a computing device in accordance with some embodiments of the present disclosure.

FIGS. 2A and 2B collectively illustrate an example flowchart of a method of classifying a subject in which dashed boxes represent optional steps in accordance with some embodiments of the present disclosure.

FIG. 3 illustrates a plot of ctDNA fraction of subjects separated by cancer type in accordance with some embodiments of the present disclosure.

FIG. 4 illustrates a plot of the ctDNA fraction of subjects with any of the cancers illustrated in FIG. 3, as a function of cancer stage in accordance with some embodiments of the present disclosure.

FIG. 5 illustrates a plot comparing the TCGA and WGBS reference sets in accordance with some embodiments of the present disclosure.

FIG. 6 illustrates that the classification method verifies patterns of differentially methylated regions in accordance with some embodiments of the present disclosure.

FIG. 7 illustrates a flowchart of a method for preparing a nucleic acid sample for sequencing in accordance with some embodiments of the present disclosure.

FIG. 8 graphical representation of the process for obtaining nucleic acid fragments in accordance with some embodiments of the present disclosure

FIG. 9 illustrates an example flowchart of a method for obtaining methylation information for the purposes of screening for a cancer condition in a test subject in accordance with some embodiments of the present disclosure

FIG. 10 provides the cumulative density function across a range of trial estimated cfDNA shedding rates in accordance with some embodiments of the present disclosure.

FIG. 11 illustrates comparing a methylation state of respective nucleic acid fragments against a first canonical set of methylation state vectors representative of a first cell source and against a second canonical set of methylation state vectors representative of a source other than the first cell source, in accordance with some embodiments of the present disclosure.

FIG. 12 illustrates transforming a plurality of first scores into a first plurality of counts, where each count in the first plurality of counts is for a methylation site in a first predetermined set of methylation sites in the genome of a reference sequence of a species, and the first predetermined set of methylation sites is associated with a first cell source in accordance with an embodiment of the present disclosure.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

The implementations described herein provide various technical solutions for determining an estimated tumor fraction of a subject. Nucleic acid fragments are obtained from a biological sample of a subject. The biological sample comprises cell-free nucleic acid. Thus, the nucleic acid fragments are cell-free nucleic acids. The nucleic acid fragments are evaluated for methylation status for a predefined set of methylation sites, and are each assigned a score based on methylation state. The plurality of methylation state scores is transformed into a plurality of counts, which are compared to a corresponding methylation score for each methylation site in the predefined set of methylation sites. The corresponding methylation scores are from analysis of methylation patterns in a first cell source. This comparison determines a frequency of methylation in the subject, which is then used to estimate tumor fraction, with regard to the first cell source.

### Definitions

As used herein, the term “about” or “approximately” mean within an acceptable error range for the particular value as determined by one of ordinary skill in the art, which depends in part on how the value is measured or determined, e.g., the limitations of the measurement system. For example, in some embodiments “about” mean within 1 or more than 1 standard deviation, per the practice in the art. In some embodiments, “about” means a range of ±20%, ±10%, ±5%, or ±1% of a given value. In some embodiments, the term “about” or “approximately” means within an order of magnitude, within 5-fold, or within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. In some embodiments, the term “about” refers to ±10%. In some embodiments, the term “about” refers to ±5%.

As used herein, the term “assay” refers to a technique for determining a property of a substance, e.g., a nucleic acid, a protein, a cell, a tissue, or an organ. An assay (e.g., a first assay or a second assay) can comprise a technique for determining the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay known to a person having ordinary skill in the art can be used to detect any of the properties of nucleic acids mentioned herein. Properties of nucleic acid molecules can include a sequence, genomic identity, copy number, methylation state at one or more nucleotide positions, size of the nucleic acid, presence or absence of a mutation in the nucleic acid at one or more nucleotide positions, and pattern of fragmentation of a nucleic acid (e.g., the nucleotide position(s) at which a nucleic acid fragments). An assay or method can have a particular sensitivity and/or specificity, and their relative usefulness as a diagnostic tool can be measured using ROC-AUC statistics. As disclosed herein, a sequencing assay can be a whole genome sequencing assay (e.g., non-methylated or methylated) or a targeted sequencing assay (e.g., non-methylated or methylated).

As used herein, the terms “biological sample,” “patient sample,” and “sample” are interchangeably used and refer to any sample taken from a subject, which can reflect a biological state associated with the subject. In some embodiments such samples contain cell-free nucleic acids such as cell-free DNA. In some embodiments, such samples include nucleic acids other than or in addition to cell-free nucleic acids. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. In some embodiments, the biological sample consists of blood, whole blood, plasma, serum, urine, cerebrospinal fluid, fecal, saliva, sweat, tears, pleural fluid, pericardial fluid, or peritoneal fluid of the subject. In such embodiments, the biological sample is limited to blood, whole blood, plasma, serum, urine, cerebrospinal fluid, fecal, saliva, sweat, tears, pleural fluid, pericardial fluid, or peritoneal fluid of the subject and does not contain other components (e.g., solid tissues, etc.) of the subject. A biological sample can include any tissue or material derived from a living or dead subject. A biological sample can be a cell-free sample. A biological sample can comprise a nucleic acid (e.g., DNA or RNA) or a fragment thereof. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis. A biological sample can be obtained from a subject invasively (e.g., surgical means) or non-invasively (e.g., a blood draw, a swab, or collection of a discharged sample).

In some embodiments, a biological sample is derived from one tissue type (e.g., from a single organ such as breast, lung, prostate, colorectal, renal, uterine, pancreatic, esophageal, lymph, ovarian, cervical, epidermal, thyroid, bladder, or gastric). In some embodiments, a biological sample is derived from one tissue type under a particular condition (e.g., a breast cancer tissue, a lung cancer tissue, a tissue of a fatty liver sample, and etc.) In some embodiments, a biological sample is derived from a two or more tissue types (e.g., a combination of tissue from two or more organs). In some embodiments, a biological sample is derived from one or more cell types (e.g., cells originating from a single organ or from a predetermined set of organs).

As disclosed herein, the terms “nucleic acid” and “nucleic acid molecule” are used interchangeably. The terms refer to nucleic acids of any composition form, such as deoxyribonucleic acid (DNA, e.g., complementary DNA (cDNA), genomic DNA (gDNA) and the like), ribonucleic acid (RNA, e.g., message RNA (mRNA), short inhibitory RNA (siRNA), ribosomal RNA (rRNA), transfer RNA (tRNA), microRNA, RNA highly expressed by the fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid in some embodiments can be from a single chromosome or fragment thereof (e.g., a nucleic acid sample may be from one chromosome of a sample obtained from a diploid organism). In certain embodiments nucleic acids comprise nucleosomes, fragments or parts of nucleosomes or nucleosome-like structures. Nucleic acids sometimes comprise protein (e.g., histones, DNA binding proteins, and the like). Nucleic acids analyzed by processes described herein sometimes are substantially isolated and are not substantially associated with protein or other molecules. Nucleic acids also include derivatives, variants and analogs of RNA or DNA synthesized, replicated or amplified from single-stranded (“sense” or “antisense,” “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. For RNA, the base cytosine is replaced with uracil and the sugar 2′ position includes a hydroxyl moiety. A nucleic acid may be prepared using a nucleic acid obtained from a subject as a template.

As used herein, the term “cell-free nucleic acids” refers to nucleic acid molecules that can be found outside cells, in bodily fluids such as blood, whole blood, plasma, serum, urine, cerebrospinal fluid, fecal, saliva, sweat, sweat, tears, pleural fluid, pericardial fluid, or peritoneal fluid of a subject. Cell-free nucleic acids originate from one or more healthy cells and/or from one or more cancer cells Cell-free nucleic acids are used interchangeably as circulating nucleic acids. Examples of the cell-free nucleic acids include but are not limited to RNA, mitochondrial DNA, or genomic DNA. As used herein, the terms “cell free nucleic acid,” “cell free DNA,” and “cfDNA” are used interchangeably. As used herein, the term “circulating tumor DNA” or “ctDNA” refers to nucleic acid fragments that originate from tumor cells or other types of cancer cells, which may be released into a fluid from an individual's body (e.g., bloodstream) as result of biological processes such as apoptosis or necrosis of dying cells or actively released by viable tumor cells. Examples of the cell-free nucleic acids include but are not limited to RNA, mitochondrial DNA, or genomic DNA.

As disclosed herein, the term “circulating tumor DNA” or “ctDNA” refers to nucleic acid fragments that originate from aberrant tissue, such as the cells of a tumor or other types of cancer, which may be released into a subject's bloodstream as results of biological processes such as apoptosis or necrosis of dying cells or actively released by viable tumor cells.

As disclosed herein, the term “reference genome” refers to any particular known, sequenced or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

As disclosed herein, the term “regions of a reference genome,” “genomic region,” or “chromosomal region” refers to any portion of a reference genome, contiguous or non-contiguous. It can also be referred to, for example, as a bin, a partition, a genomic portion, a portion of a reference genome, a portion of a chromosome and the like. In some embodiments, a genomic section is based on a particular length of genomic sequence. In some embodiments, a method can include analysis of multiple mapped nucleic acid fragments to a plurality of genomic regions. Genomic regions can be approximately the same length or the genomic sections can be different lengths. In some embodiments, genomic regions are of about equal length. In some embodiments genomic regions of different lengths are adjusted or weighted. In some embodiments, a genomic region is about 10 kilobases (kb) to about 500 kb, about 20 kb to about 400 kb, about 30 kb to about 300 kb, about 40 kb to about 200 kb, and sometimes about 50 kb to about 100 kb. In some embodiments, a genomic region is about 100 kb to about 200 kb. A genomic region is not limited to contiguous runs of sequence. Thus, genomic regions can be made up of contiguous and/or non-contiguous sequences. A genomic region is not limited to a single chromosome. In some embodiments, a genomic region includes all or part of one chromosome or all or part of two or more chromosomes. In some embodiments, genomic regions may span one, two, or more entire chromosomes. In addition, the genomic regions may span joint or disjointed portions of multiple chromosomes.

As used herein, the term “fragment” is used interchangeably with “nucleic acid fragment” (e.g., a DNA fragment), and refers to a portion of a polynucleotide or polypeptide sequence that comprises at least three consecutive nucleotides. In the context of sequencing of cell-free nucleic acid molecules found in a biological sample, the terms “fragment” and “nucleic acid fragment” interchangeably refer to a cell-free nucleic acid molecule that is found in the biological sample or a representation thereof. In such a context, sequencing data (e.g., sequence reads from whole genome sequencing, targeted sequencing, etc.) are used to derive one or more copies of all or a portion of such a nucleic acid fragment. As disclosed herein, methylation status information can be obtained in connection with either whole genome or targeted methylation sequencing. Such sequence reads, which in fact may be obtained from sequencing of PCR duplicates of the original nucleic acid fragment, therefore “represent” or “support” the nucleic acid fragment. There may be a plurality of sequence reads that each represent or support a particular nucleic acid fragment in the biological sample (e.g., PCR duplicates). In some embodiments, nucleic acid fragments can be considered cell-free nucleic acids. In some embodiments, sequence reads from PCR duplicates can be misleading; for example, when the abundance level of a particular cell-free nucleic acid molecule needs to be determined. In such embodiments, only one copy of a nucleic acid fragment is used to represent the original cell-free nucleic acid molecule (e.g., duplicates are removed through molecular identifiers that are attached to the cell-free nucleic acid molecule during the library preparation process). In some embodiments, methylation sequencing data can be used to further distinguish these nucleic acid fragments. For example, two nucleic acid fragments that share identical or near identical sequences may still correspond to different original cell-free nucleic acid molecules if they each harbor a different methylation pattern. As disclosed here, nucleic acid fragments are defined based on sequence information and methylation status embedded therein. One of skill in the art would understand that fragment identification and subsequent analysis can be performed regardless of whether the initial sequencing assay targets the entire genome (e.g., whole genome methylation sequencing) or only selected regions of the genome (e.g., targeted methylation sequencing).

In some embodiments, two fragments are considered to share near identical nucleic acid sequences when the respective fragment sequences differ from each other by fewer than 2 nucleotides, by fewer than 3 nucleotides, by fewer than 4 nucleotides, by fewer than 5 nucleotides, by fewer than 6 nucleotides, by fewer than 7 nucleotides, by fewer than 8 nucleotides, by fewer than 9 nucleotides, by fewer than 10 nucleotides, by fewer than 15 nucleotides, by fewer than 20 nucleotides, by fewer than 25 nucleotides, by fewer than 30 nucleotides, by fewer than 35 nucleotides, by fewer than 40 nucleotides, by fewer than 45 nucleotides, or by fewer than 50 nucleotides. In some embodiments, two fragments are considered to share near identical sequences when the respective fragment sequences differ from each other by less than 1% of the total nucleotides, by less than 2% of the total nucleotides, by less than 3% of the total nucleotides, by less than 4% of the total nucleotides, or by less than 5% of the total nucleotides.

In some embodiments, a first fragment from a respective (e.g., a first or second) plurality of nucleic acid fragments is aligned to a first location in a reference genome and a second fragment from the respective (e.g., the first or second) plurality of nucleic acid fragments is aligned to a second location in a reference genome. In some embodiments, the first and second location correspond to distinct regions in the reference genome. In some embodiments, the first and second locations are the same location (e.g., the first and second locations correspond to the same region of the reference genome). In some embodiments, the first and second locations overlap in the reference genome by at least 1 residue, at least 2 residues, at least 3 residues, at least 4 residues, at least 5 residues, at least 6 residues, at least 7 residues, at least 8 residues, at least 9 residues, at least 10 residues, by at least 11 residues, by at least 12 residues, by at least 13 residues, by at least 14 residues, by at least 15 residues, by at least 16 residues, by at least 17 residues, by at least 18 residues, by at least 19 residues, by at least 20 residues, by at least 30 residues, by at least 40 residues, by at least 50 residues, by at least 60 residues, by at least 70 residues, by at least 80 residues, by at least 90 residues, or by at least 100 residues. In some embodiments, the first and second location overlap in the reference genome by between 1 and 50 residues. In some embodiments, the first and second location map to different genes in the reference genome. In some embodiments, the first and second locations are on different chromosomes of the reference genome.

As used herein, the term “sequence reads” or “reads” refers to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). In some embodiments, sequence reads (e.g., single-end or paired-end reads) can be generated from one or both strands of a targeted nucleic acid fragment. The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As disclosed herein, the terms “sequencing,” “sequence determination,” and the like as used herein refers generally to any and all biochemical processes that may be used to determine the order of biological macromolecules such as nucleic acids or proteins. For example, sequencing data can include all or a portion of the nucleotide bases in a nucleic acid molecule such as a DNA fragment.

As disclosed herein, the term “single nucleotide variant” or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.”

As used herein, the term “methylation profile” (also called methylation status) can include information related to DNA methylation for a region. Information related to DNA methylation can include a methylation index of a CpG site, a methylation density of CpG sites in a region, a distribution of CpG sites over a contiguous region, a pattern or level of methylation for each individual CpG site within a region that contains more than one CpG site, and non-CpG methylation. A methylation profile of a substantial part of the genome can be considered equivalent to the methylome. “DNA methylation” in mammalian genomes can refer to the addition of a methyl group to position 5 of the heterocyclic ring of cytosine (e.g., to produce 5-methylcytosine) among CpG dinucleotides. Methylation of cytosine can occur in cytosines in other sequence contexts, for example 5′-CHG-3′ and 5′-CHH-3′, where H is adenine, cytosine or thymine. Cytosine methylation can also be in the form of 5-hydroxymethylcytosine. Methylation of DNA can include methylation of non-cytosine nucleotides, such as N6-methyladenine.

As used herein a “methylome” can be a measure of an amount of DNA methylation at a plurality of sites or loci in a genome. The methylome can correspond to all of a genome, a substantial part of a genome, or relatively small portion(s) of a genome. A “tumor methylome” can be a methylome of a tumor of a subject (e.g., a human). A tumor methylome can be determined using tumor tissue or cell-free tumor DNA in plasma. A tumor methylome can be one example of a methylome of interest. A methylome of interest can be a methylome of an organ that can contribute nucleic acid, e.g., DNA into a bodily fluid (e.g., a methylome of brain cells, a bone, lungs, heart, muscles, kidneys, etc.). The organ can be a transplanted organ.

As used herein the term “methylation index” for each genomic site (e.g., a CpG site, a region of DNA where a cytosine nucleotide is followed by a guanine nucleotide in the linear sequence of bases along its 5′→3′ direction) can refer to the proportion of nucleic acid fragments showing methylation at the site over the total number of nucleic acid fragments covering that site. The “methylation density” of a region can be the number of nucleic acid fragments at sites within a region showing methylation divided by the total number of nucleic acid fragments covering the sites in the region. The sites can have specific characteristics, (e.g., the sites can be CpG sites). The “CpG methylation density” of a region can be the number of nucleic acid fragments showing CpG methylation divided by the total number of nucleic acid fragments covering CpG sites in the region (e.g., a particular CpG site, CpG sites within a CpG island, or a larger region). For example, the methylation density for each 100-kb bin in the human genome can be determined from the total number of unconverted cytosines (which can correspond to methylated cytosine) at CpG sites as a proportion of all CpG sites covered by nucleic acid fragments mapped to the 100-kb region. In some embodiments, this analysis is performed for other bin sizes, e.g., 50-kb or 1-Mb, etc. In some embodiments, a region is an entire genome or a chromosome or part of a chromosome (e.g., a chromosomal arm). A methylation index of a CpG site can be the same as the methylation density for a region when the region only includes that CpG site. The “proportion of methylated cytosines” can refer the number of cytosine sites, “C's,” that are shown to be methylated (for example unconverted after bisulfite conversion) over the total number of analyzed cytosine residues, e.g., including cytosines outside of the CpG context, in the region. The methylation index, methylation density and proportion of methylated cytosines are examples of “methylation levels.”

As used herein, the term “relative abundance” can refer to a ratio of a first amount of nucleic acid fragments having a particular characteristic (e.g., a specified length, ending at one or more specified coordinates/ending positions, aligning to a particular region of the genome, or having a particular methylation status) to a second amount nucleic acid fragments having a particular characteristic (e.g., a specified length, ending at one or more specified coordinates/ending positions, aligning to a particular region of the genome, or having a particular methylation status). In one example, relative abundance may refer to a ratio of the number of DNA fragments ending at a first set of genomic positions to the number of DNA fragments ending at a second set of genomic positions. In some aspects, a “relative abundance” can be a type of separation value that relates an amount (one value) of cell-free DNA molecules ending within one window of genomic position to an amount (other value) of cell-free DNA molecules ending within another window of genomic positions. The two windows can overlap, but can be of different sizes. In other embodiments, the two windows cannot overlap. Further, in some embodiments, the windows are of a width of one nucleotide, and therefore are equivalent to one genomic position.

As used herein, the term “methylation” refers to a modification of deoxyribonucleic acid (DNA) where a hydrogen atom on the pyrimidine ring of a cytosine base is converted to a methyl group, forming 5-methylcytosine. In particular, methylation tends to occur at dinucleotides of cytosine and guanine referred to herein as “CpG sites”. In other instances, methylation may occur at a cytosine not part of a CpG site or at another nucleotide that's not cytosine; however, these are rarer occurrences. In this present disclosure, methylation is discussed in reference to CpG sites for the sake of clarity. Anomalous cfDNA methylation can identified as hypermethylation or hypomethylation, both of which may be indicative of cancer status. As is well known in the art, DNA methylation anomalies (compared to healthy controls) can cause different effects, which may contribute to cancer.

Various challenges arise in the identification of anomalously methylated cfDNA fragments. First, determining a subject's cfDNA to be anomalously methylated only holds weight in comparison with a group of control subjects, such that if the control group is small in number, the determination loses confidence with the small control group. Additionally, among a group of control subjects' methylation status can vary which can be difficult to account for when determining a subject's cfDNA to be anomalously methylated. On another note, methylation of a cytosine at a CpG site causally influences methylation at a subsequent CpG site.

Those of skill in the art will appreciate that the principles described herein are equally applicable for the detection of methylation in a non-CpG context, including non-cytosine methylation. Further, the methylation state vectors may contain elements that are generally vectors of sites where methylation has or has not occurred (even if those sites are not CpG sites specifically). With that substitution, the remainder of the processes described herein are the same, and consequently the inventive concepts described herein are applicable to those other forms of methylation.

As disclosed herein, the term “subject” refers to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. The terms “subject” and “patient” are used interchangeably herein and refer to a human or non-human animal who is known to have, or potentially has, a medical condition or disorder, such as, e.g., a cancer. In some embodiments, a subject is a male or female of any stage (e.g., a man, a woman, or a child).

A subject from whom a sample is taken, or is treated by any of the methods or compositions described herein can be of any age and can be an adult, infant or child. In some cases, the subject, e.g., patient is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 years old, or within a range therein (e.g., between about 2 and about 20 years old, between about 20 and about 40 years old, or between about 40 and about 90 years old). A particular class of subjects, e.g., patients that can benefit from a method of the present disclosure is subjects, e.g., patients over the age of 40.

Another particular class of subjects, e.g., patients that can benefit from a method of the present disclosure is pediatric patients, who can be at higher risk of chronic heart symptoms. Furthermore, a subject, e.g., patient from whom a sample is taken, or is treated by any of the methods or compositions described herein, can be male or female.

The term “normalize” as used herein means transforming a value or a set of values to a common frame of reference for comparison purposes. For example, when a diagnostic ctDNA level is “normalized” with a baseline ctDNA level, the diagnostic ctDNA level is compared to the baseline ctDNA level so that the amount by which the diagnostic ctDNA level differs from the baseline ctDNA level can be determined.

As used herein the term “cancer” or “tumor” refers to an abnormal mass of tissue in which the growth of the mass surpasses and is not coordinated with the growth of normal tissue. A cancer or tumor can be defined as “benign” or “malignant” depending on the following characteristics: degree of cellular differentiation including morphology and functionality, rate of growth, local invasion and metastasis. A “benign” tumor can be well differentiated, have characteristically slower growth than a malignant tumor and remain localized to the site of origin. In addition, in some cases a benign tumor does not have the capacity to infiltrate, invade or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites.

As used herein, the term “level of cancer” refers to whether cancer exists (e.g., presence or absence), a stage of a cancer, a size of tumor, presence or absence of metastasis, the total tumor burden of the body, and/or other measure of a severity of a cancer (e.g., recurrence of cancer). The level of cancer can be a number or other indicia, such as symbols, alphabet letters, and colors. The level can be zero. The level of cancer can also include premalignant or precancerous conditions (states) associated with mutations or a number of mutations. The level of cancer can be used in various ways. For example, screening can check if cancer is present in someone who is not known previously to have cancer. Assessment can investigate someone who has been diagnosed with cancer to monitor the progress of cancer over time, study the effectiveness of therapies or to determine the prognosis. In one embodiment, the prognosis can be expressed as the chance of a subject dying of cancer, or the chance of the cancer progressing after a specific duration or time, or the chance of cancer metastasizing. Detection can comprise ‘screening’ or can comprise checking if someone, with suggestive features of cancer (e.g., symptoms or other positive tests), has cancer.

The terms “cancer load,” “tumor load,” “cancer burden” and “tumor burden” are used interchangeably herein to refer to a concentration or presence of tumor-derived nucleic acids in a test sample. As such, the terms “cancer load,” “tumor load,” “cancer burden” and “tumor burden” are non-limiting examples of a cell source fraction (e.g., tumor fraction) in a biological sample. In some embodiments, tumor fraction is a specific version of cell source fraction.

As used herein, the term “tissue” corresponds to a group of cells that group together as a functional unit. More than one type of cell can be found in a single tissue. Different types of tissue may consist of different types of cells (e.g., hepatocytes, alveolar cells or blood cells), but also can correspond to tissue from different organisms (mother vs. fetus) or to healthy cells vs. tumor cells. The term “tissue” can generally refer to any group of cells found in the human body (e.g., heart tissue, lung tissue, kidney tissue, nasopharyngeal tissue, oropharyngeal tissue). In some aspects, the term “tissue” or “tissue type” can be used to refer to a tissue from which a cell-free nucleic acid originates. In one example, viral nucleic acid fragments can be derived from blood tissue. In another example, viral nucleic acid fragments can be derived from tumor tissue.

As used herein the term “untrained classifier” refers to a classifier that has not been trained on a target dataset. For instance, consider the case of a first canonical set of methylation state vectors and a second canonical set of methylation state vectors discussed below. The respective canonical sets of methylation state vectors are applied as collective input to an untrained classifier, in conjunction with the cell source of each respective reference subject represented by the first canonical set of methylation state vectors (hereinafter “primary training dataset”) to train the untrained classifier on cell source thereby obtaining a trained classifier. Moreover, it will be appreciated that the term “untrained classifier” does not exclude the possibility that transfer learning techniques are used in such training of the untrained classifier. For instance, Fernandes et al., 2017, “Transfer Learning with Partial Observability Applied to Cervical Cancer Screening,” Pattern Recognition and Image Analysis: 8^{th} Iberian Conference Proceedings, 243-250, which is hereby incorporated by reference, provides non-limiting examples of such transfer learning. In instances where transfer learning is used, the untrained classifier described above is provided with additional data over and beyond that of the primary training dataset. That is, in non-limiting examples of transfer learning embodiments, the untrained classifier receives (i) canonical sets of methylation state vectors and the cell source labels of each of the reference subjects represented by canonical sets of methylation state vectors (“primary training dataset”) and (ii) additional data. Typically, this additional data is in the form of coefficients (e.g., regression coefficients) that were learned from another, auxiliary training dataset. Moreover, while a description of a single auxiliary training dataset has been disclosed, it will be appreciated that there is no limit on the number of auxiliary training datasets that may be used to complement the primary training dataset in training the untrained classifier in the present disclosure. For instance, in some embodiments, two or more auxiliary training datasets, three or more auxiliary training datasets, four or more auxiliary training datasets or five or more auxiliary training datasets are used to complement the primary training dataset through transfer learning, where each such auxiliary dataset is different than the primary training dataset. Any manner of transfer learning may be used in such embodiments. For instance, consider the case where there is a first auxiliary training dataset and a second auxiliary training dataset in addition to the primary training dataset. The coefficients learned from the first auxiliary training dataset (by application of a classifier such as regression to the first auxiliary training dataset) may be applied to the second auxiliary training dataset using transfer learning techniques (e.g., the above described two-dimensional matrix multiplication), which in turn may result in a trained intermediate classifier whose coefficients are then applied to the primary training dataset and this, in conjunction with the primary training dataset itself, is applied to the untrained classifier. Alternatively, a first set of coefficients learned from the first auxiliary training dataset (by application of a classifier such as regression to the first auxiliary training dataset) and a second set of coefficients learned from the second auxiliary training dataset (by application of a classifier such as regression to the second auxiliary training dataset) may each individually be applied to a separate instance of the primary training dataset (e.g., by separate independent matrix multiplications) and both such applications of the coefficients to separate instances of the primary training dataset in conjunction with the primary training dataset itself (or some reduced form of the primary training dataset such as principal components or regression coefficients learned from the primary training set) may then be applied to the untrained classifier in order to train the untrained classifier. In either example, knowledge regarding cell source (e.g., cancer type, etc.) derived from the first and second auxiliary training datasets is used, in conjunction with the cell source labeled primary training dataset), to train the untrained classifier.

The term “classification” can refer to any number(s) or other characters(s) that are associated with a particular property of a sample. For example, a “+” symbol (or the word “positive”) can signify that a sample is classified as having deletions or amplifications. In another example, the term “classification” refers to an amount of tumor tissue in the subject and/or sample, a size of the tumor in the subject and/or sample, a stage of the tumor in the subject, a tumor load in the subject and/or sample, and presence of tumor metastasis in the subject. In some embodiments, the classification is binary (e.g., positive or negative) or has more levels of classification (e.g., a scale from 1 to 10 or 0 to 1). In some embodiments, the terms “cutoff” and “threshold” refer to predetermined numbers used in an operation. In one example, a cutoff size refers to a size above which fragments are excluded. In some embodiments, a threshold value is a value above or below which a particular classification applies. Either of these terms can be used in either of these contexts.

As used herein, the term “cancer-associated changes” or “cancer-specific changes” can include cancer-derived mutations (including single nucleotide mutations, deletions or insertions of nucleotides, deletions of genetic or chromosomal segments, translocations, inversions), amplification of genes, virus-associated sequences (e.g., viral episomes, viral insertions, viral DNA that is infected into a cell and subsequently released by the cell, and circulating or cell-free viral DNA), aberrant methylation profiles or tumor-specific methylation signatures, aberrant cell-free nucleic acid (e.g., DNA) size profiles, aberrant histone modification marks and other epigenetic modifications, and locations of the ends of cell-free DNA fragments that are cancer-associated or cancer-specific.

As used herein, the terms “control,” “control sample,” “reference,” “reference sample,” “normal,” and “normal sample” describe a sample from a subject that does not have a particular condition, or is otherwise healthy. In an example, a method as disclosed herein can be performed on a subject having a tumor, where the reference sample is a sample taken from a healthy tissue of the subject. A reference sample can be obtained from the subject, or from a database. The reference can be, e.g., a reference genome that is used to map nucleic acid fragments obtained from sequencing a sample from the subject. A reference genome can refer to a haploid or diploid genome to which nucleic acid fragment from the biological sample and a constitutional sample can be aligned and compared. An example of constitutional sample can be DNA of white blood cells obtained from the subject. For a haploid genome, there can be only one nucleotide at each locus. For a diploid genome, heterozygous loci can be identified; each heterozygous locus can have two alleles, where either allele can allow a match for alignment to the locus.

The terminology used herein is for the purpose of describing particular cases only and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

Exemplary System Embodiments

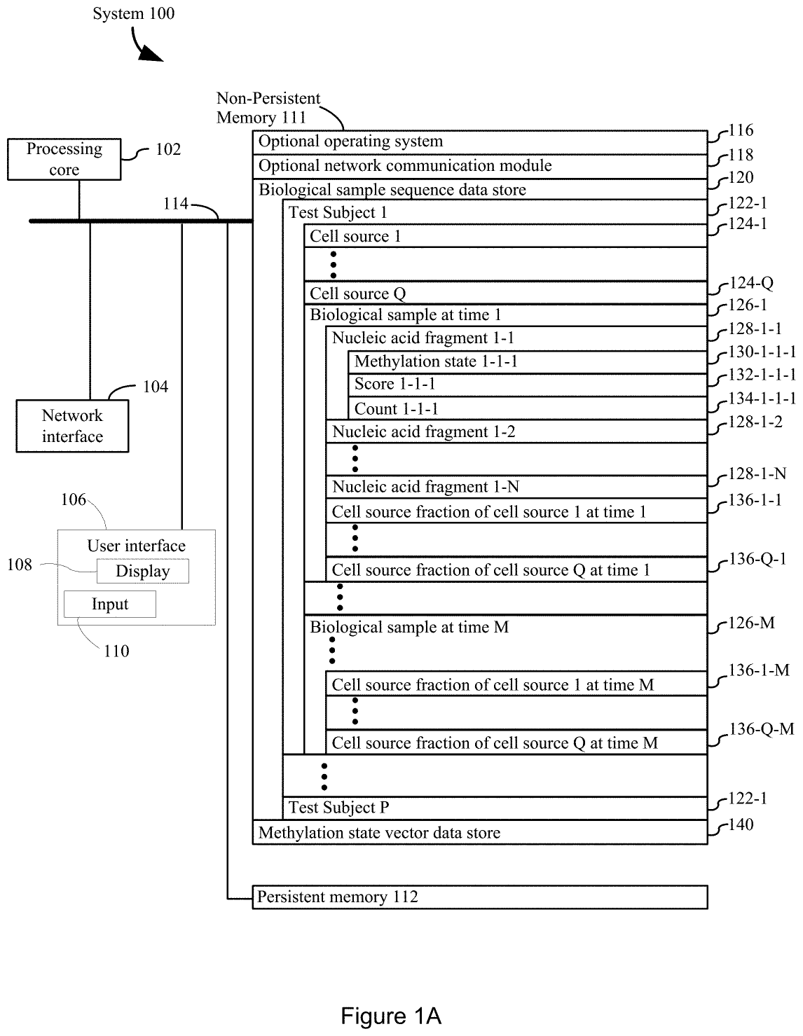

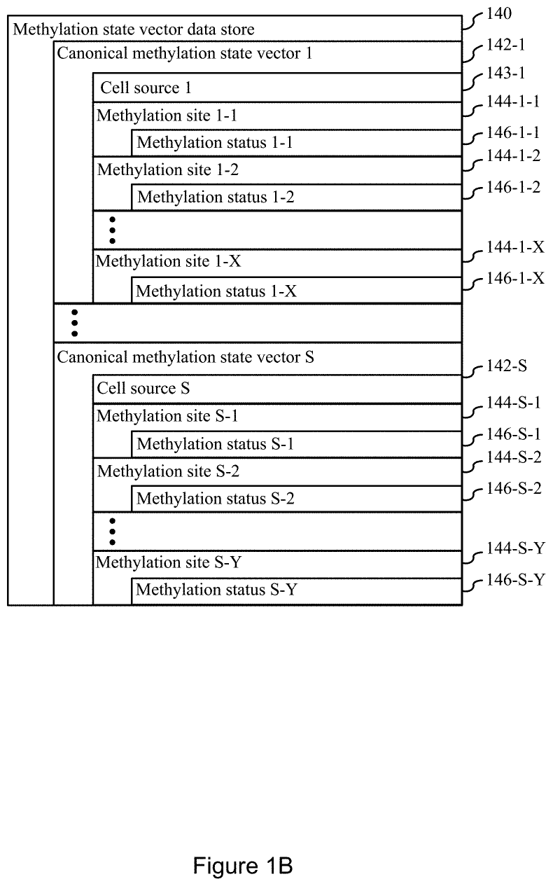

Details of an exemplary system are now described in conjunction with FIG. 1. FIG. 1 is a block diagram illustrating system 100 in accordance with some implementations. Device 100 in some implementations includes one or more processing units CPU(s) 102 (also referred to as processors or processing core), one or more network interfaces 104, user interface 106, non-persistent memory 111, persistent memory 112, and one or more communication buses 114 for interconnecting these components. One or more communication buses 114 optionally include circuitry (sometimes called a chipset) that interconnects and controls communications between system components. Non-persistent memory 111 typically includes high-speed random access memory, such as DRAM, SRAM, DDR RAM, ROM, EEPROM, flash memory, whereas persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. Persistent memory 112 optionally includes one or more storage devices remotely located from the CPU(s) 102. Persistent memory 112, and the non-volatile memory device(s) within non-persistent memory 112, comprise non-transitory computer readable storage medium. In some implementations, non-persistent memory 111 or alternatively non-transitory computer readable storage medium stores the following programs, modules and data structures, or a subset thereof, sometimes in conjunction with persistent memory 112:
optional operating system 116, which includes procedures for handling various basic system services and for performing hardware dependent tasks;
optional network communication module (or instructions) 118 for connecting the system 100 with other devices, or a communication network;
a biological sample sequence data store 120 for determining a cell source fraction 136 of a test subject 122 in a biological sample collected at a time 126;
information for each respective test subject 122 including (i) possible cell sources 124 of the at least one biological sample collected at a time 126 for the respective subject, where each biological sample comprises at least one nucleic acid fragment 128, and where information for each nucleic acid fragment includes (i) at least one methylation state 130, (ii) a score 132, and (iii) optionally a count 134; and
a methylation state vector data store 140 that comprises one or more canonical methylation states vectors 142, each methylation state vector comprising a cell source 143 and a plurality of methylation sites 144 (e.g., genomic locations for methylation sites in a reference genome), each methylation site with a corresponding methylation status 146.

In some implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above identified elements is stored in a computer system, other than that of visualization system 100, that is addressable by visualization system 100 so that visualization system 100 may retrieve all or a portion of such data when needed.

Although FIG. 1 depicts a “system 100,” the figure is intended more as a functional description of the various features which may be present in computer systems than as a structural schematic of the implementations described herein. In practice, and as recognized by those of ordinary skill in the art, items shown separately could be combined and some items could be separated. Moreover, although FIG. 1 depicts certain data and modules in non-persistent memory 111, some or all of these data and modules may be in persistent memory 112.

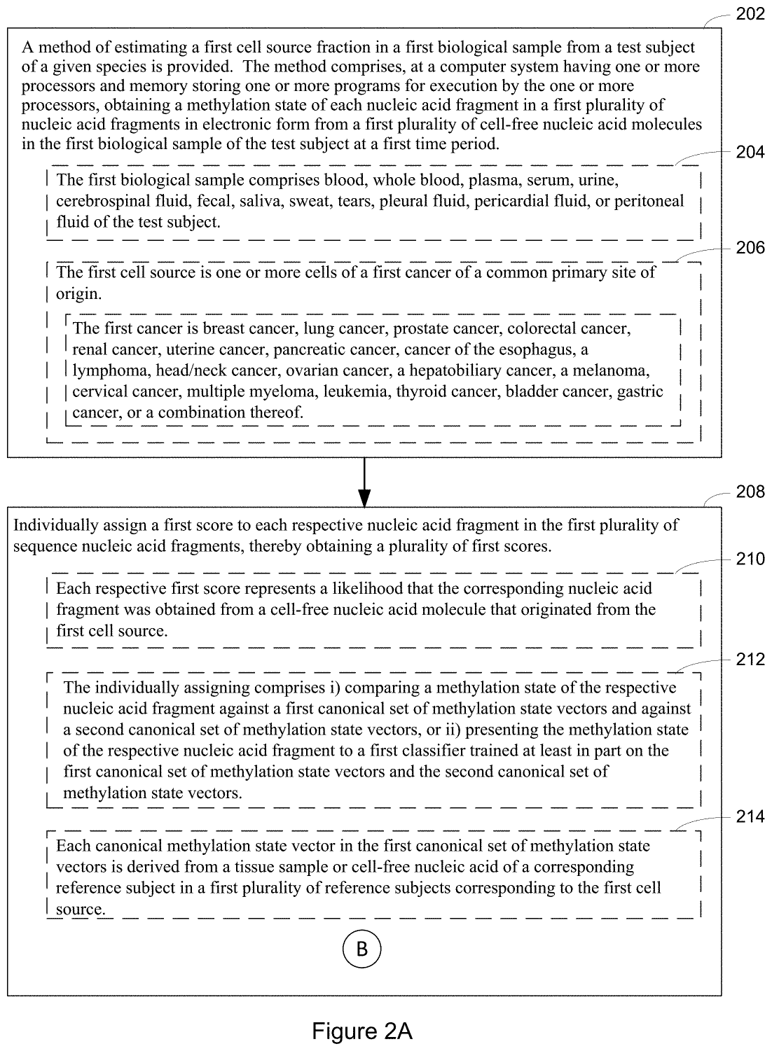

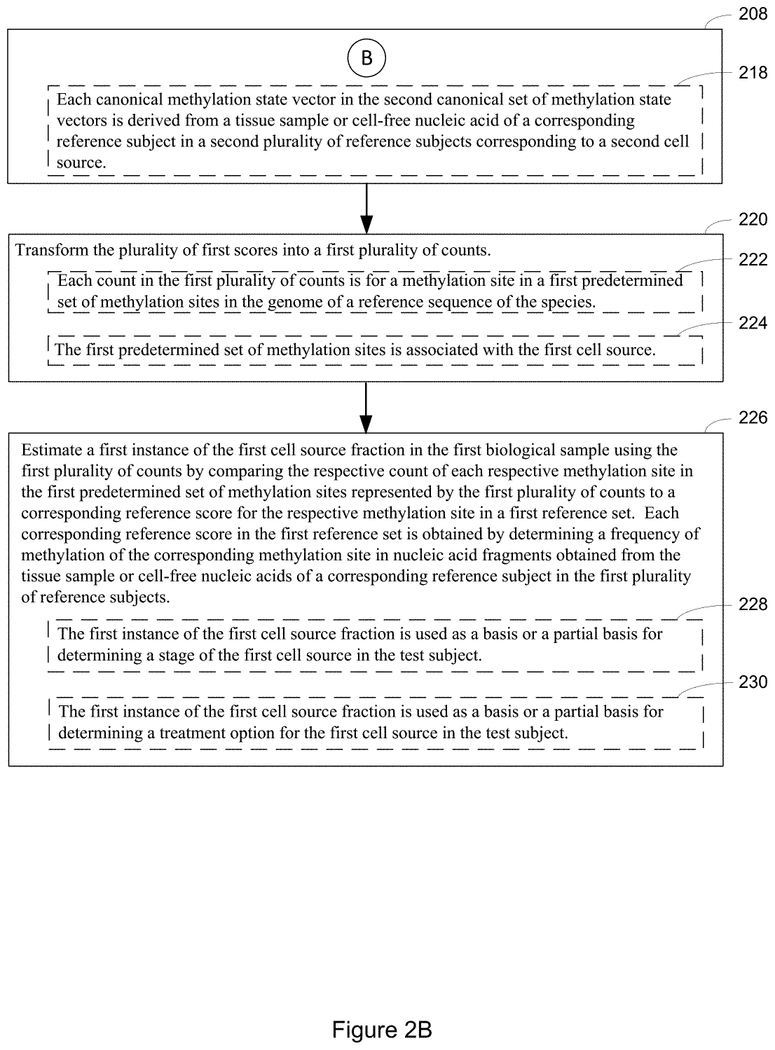

While a system in accordance with the present disclosure has been disclosed with reference to FIG. 1, methods in accordance with the present disclosure are now detailed with reference to FIG. 2. It will be appreciated that any of the disclosed methods can make use of any of the assays or algorithms disclosed in U.S. patent application Ser. No. 15/793,830, filed Oct. 25, 2017 and/or International Patent Publication No. PCT/US17/58099, having an International Filing Date of Oct. 24, 2017, each of which is hereby incorporated by reference, in order to determine a cancer condition in a test subject or a likelihood that the subject has the cancer condition. For instance, any of the disclosed methods can work in conjunction with any of the disclosed methods or algorithms disclosed in U.S. patent application Ser. No. 15/793,830, filed Oct. 25, 2017, and/or International Patent Publication No. PCT/US17/58099, having an International Filing Date of Oct. 24, 2017.

Determining an estimated first cell source fraction for a test subject with respect to a first condition.

Block 202. A method of estimating a first cell source fraction in a first biological sample from a test subject of a given species is provided. In some embodiments, the test subject is a human subject. In some embodiments, the test subject is a mammalian. Using computer system 100 there is obtained a methylation state 130 of each nucleic acid fragment 128 in a first plurality of nucleic acid fragments in electronic form from a first plurality of cell-free nucleic acid molecules in a first biological sample of the test subject at a first time period 126. The methylation state of each nucleic acid fragment 128 is in fact inferred from that portion of the sequence of each nucleic acid fragment that is mappable to a reference genome as discussed in more detail below. In some embodiments, nucleic acid fragments are obtained as discussed in Example 2 below.

In some embodiments, the subject is any living or non-living organism, including but not limited to a human (e.g., a male human, female human, fetus, pregnant female, child, or the like), a non-human animal, a plant, a bacterium, a fungus or a protist. In some embodiments, the subject is a mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any stage (e.g., a man, a women or a child).

In some embodiments, the biological sample comprises blood, whole blood, plasma, serum, urine, cerebrospinal fluid, fecal, saliva, sweat, tears, pleural fluid, pericardial fluid, or peritoneal fluid of the subject. In such embodiments, the biological sample may include the blood, whole blood, plasma, serum, urine, cerebrospinal fluid, fecal, saliva, sweat, tears, pleural fluid, pericardial fluid, or peritoneal fluid of the subject as well as other components (e.g., solid tissues, etc.) of the subject.

In some embodiments, the biological sample consists of blood, whole blood, plasma, serum, urine, cerebrospinal fluid, fecal, saliva, sweat, tears, pleural fluid, pericardial fluid, or peritoneal fluid of the subject. In such embodiments, the biological sample is limited to blood, whole blood, plasma, serum, urine, cerebrospinal fluid, fecal, saliva, sweat, tears, pleural fluid, pericardial fluid, or peritoneal fluid of the subject and does not contain other components (e.g., solid tissues, etc.) of the subject.

In some embodiments, the biological sample comprises or consists of one or more specific cell types (e.g., the biological sample is derived from one or more cell types). In some embodiments, the one or more cell types comprise a combination of healthy, non-cancerous cells and cancerous cells.

Such biological samples contain cell-free nucleic acid fragments (e.g., cfDNA fragments). In some embodiments, the biological sample is processed to extract the cell-free nucleic acids in preparation for sequencing analysis. By way of a non-limiting example, in some embodiments, cell-free nucleic acid fragments are extracted from a biological sample (e.g., blood sample) collected from a subject in K2 EDTA tubes. In the case where the biological samples are blood, the samples are processed within two hours of collection by double spinning of the biological sample first at ten minutes at 1000 g, and then the resulting plasma is spun ten minutes at 2000 g. The plasma is then stored in 1 ml aliquots at −80° C. In this way, a suitable amount of plasma (e.g. 1-5 ml) is prepared from the biological sample for the purposes of cell-free nucleic acid extraction. In some such embodiments cell-free nucleic acid is extracted using the QIAamp Circulating Nucleic Acid kit (Qiagen) and eluted into DNA Suspension Buffer (Sigma). In some embodiments, the purified cell-free nucleic acid is stored at −20° C. until use. See, for example, Swanton, et al., 2017, “Phylogenetic ctDNA analysis depicts early stage lung cancer evolution,” Nature, 545(7655): 446-451, which is hereby incorporated by reference. Other equivalent methods can be used to prepare cell-free nucleic acid from biological methods for the purpose of sequencing, and all such methods are within the scope of the present disclosure.

In some embodiments, the cell-free nucleic acid fragments that are obtained from a biological sample are any form of nucleic acid defined in the present disclosure, or a combination thereof. For example, in some embodiments, the cell-free nucleic acid that is obtained from a biological sample is a mixture of RNA and DNA.

In some embodiments, the cell-free nucleic acid fragments are treated to convert unmethylated cytosines to uracils. In one embodiment, the method uses a bisulfite treatment of the DNA that converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, Calif.)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, Mass.).

From the converted cell-free nucleic acid fragments, a sequencing library is prepared. Optionally, the sequencing library is enriched for cell-free nucleic acid fragments, or genomic regions, that are informative for cell origin using a plurality of hybridization probes. The hybridization probes are short oligonucleotides that hybridize to particularly specified cell-free nucleic acid fragments, or targeted regions, and enrich for those fragments or regions for subsequent sequencing and analysis. In some embodiments, hybridization probes are used to perform a targeted, high-depth analysis of a set of specified CpG sites that are informative for cell origin. Once prepared, the sequencing library or a portion thereof is sequenced to obtain a plurality of nucleic acid fragments.

In this way, in some embodiments, more than 1000 nucleic acid fragments 128 are recovered from the biological sample. In some embodiments, more than 5000 nucleic acid fragments 128 are recovered from the biological sample. In some embodiments, more than 10,000, 100,000, 200,000, 300,000, 400,000, 500,000, 600,000, 700,000, 800,000, 900,000, 1 million, 2 million, 3 million, 4 million, 5 million, 6 million, 7 million, 8 million, 9 million, 10 million, 15 million or 20 million nucleic acid fragments 128 are recovered from the biological sample. In some embodiments, the nucleic acid fragments 128 recovered from the biological sample are based on nucleic acid sequencing that provides a coverage rate of 1× or greater, 2× or greater, 5× or greater, 10× or greater, or 50× or greater for at least two percent, at least five percent, at least ten percent, at least twenty percent, at least thirty percent, at least forty percent, at least fifty percent, at least sixty percent, at least seventy percent, at least eighty percent, at least ninety percent, at least ninety-eight percent, or at least ninety-nine percent of the genome of the subject.

Any form of sequencing can be used to obtain the nucleic acid fragments 128 from the cell-free nucleic acid obtained from the biological sample including, but not limited to, high-throughput sequencing systems such as the Roche 454 platform, the Applied Biosystems SOLID platform, the Helicos True Single Molecule DNA sequencing technology, the sequencing-by-hybridization platform from Affymetrix Inc., the single molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and nanopore sequencing also can be used to obtain nucleic acid fragments 128 from the cell-free nucleic acid obtained from the biological sample.

In some embodiments, sequencing-by-synthesis and reversible terminator-based sequencing (e.g., Illumina's Genome Analyzer; Genome Analyzer II; HISEQ 2000; HISEQ 2500 (Illumina, San Diego Calif.)) is used to obtain nucleic acid fragments 128 from the cell-free nucleic acid obtained from the biological sample. In some such embodiments, millions of cell-free nucleic acid (e.g., DNA) fragments are sequenced in parallel. In one example of this type of sequencing technology, a flow cell is used that contains an optically transparent slide with eight individual lanes on the surfaces of which are bound oligonucleotide anchors (e.g., adaptor primers). A flow cell often is a solid support that is configured to retain and/or allow the orderly passage of reagent solutions over bound analytes. In some instance, flow cells are planar in shape, optically transparent, generally in the millimeter or sub-millimeter scale, and often have channels or lanes in which the analyte/reagent interaction occurs. In some embodiments, a cell-free nucleic acid sample can include a signal or tag that facilitates detection. In some such embodiments, the acquisition of nucleic acid fragments 128 from the cell-free nucleic acid obtained from the biological sample includes obtaining quantification information of the signal or tag via a variety of techniques such as, for example, flow cytometry, quantitative polymerase chain reaction (qPCR), gel electrophoresis, gene-chip analysis, microarray, mass spectrometry, cytofluorimetric analysis, fluorescence microscopy, confocal laser scanning microscopy, laser scanning cytometry, affinity chromatography, manual batch mode separation, electric field suspension, sequencing, and combination thereof.

In some embodiments, the nucleic acid fragments are corrected for background copy number. For instance, nucleic acid fragments that arise from chromosomes or portions of chromosomes that are duplicated in the subject are corrected for this duplication. This can be done either by normalizing before running this inference, or allowing for more than one value of first cell source fraction. Allowing for more than one first cell source fraction also enables assessment of heterogeneity within a test subject. As such, in some embodiments, the assumption that each nucleic acid fragment represents an independent observation of the single estimated first cell source fraction is corrected for background copy number.

In some embodiments the plurality of nucleic acid fragments 128, obtained from cell-free nucleic acid sample of a biological sample, comprises more than ten, one hundred, five hundred, one thousand, two thousand, five thousand, ten thousand, 100,000, 200,000, 300,000, 400,000, 500,000, 600,000, 700,000, 800,000, 900,000, 1 million, 2 million, 3 million, 4 million, 5 million, 6 million, 7 million, 8 million, 9 million, 10 million, 15 million or 20 million nucleic acid fragments of the cell-free nucleic acid. In some embodiments, each of these nucleic acid fragments is of a different portion of the cell-free nucleic acid. In some embodiments one nucleic acid fragment 128 in the first plurality of nucleic acid fragments maps to the same over overlapping portion of a reference genome as another nucleic acid fragment in the first plurality of nucleic acid fragments.

In some embodiments, each nucleic acid fragment represents a different cell-free nucleic acid fragment. In such instances, the coverage of the cell-free nucleic acid fragments is deemed to be 1 because of the 1 to 1 relationship.

In some embodiments, on average, each cell-free nucleic acid fragment in the plurality of nucleic acid fragments is represented by two different sequence reads. In such instances, the coverage of the cell-free nucleic acid fragments is deemed to be 2 because of the 2 to 1 relationship between sequence reads and the cell-free nucleic acid fragments. In other words, when coverage is 2, for each respective cell-free nucleic acid fragment represented by the plurality of nucleic acid fragments, there will be, on average, two different sequence reads from the nucleic acid sequencing that map onto the respective cell-free nucleic acid fragment.

In some embodiments, on average, each cell-free nucleic acid fragment in the plurality of nucleic acid fragments is represented by three, four, five, six, seven, eight, nine, or ten different sequence reads from the nucleic acid sequencing. In such instances, the coverage of the cell-free nucleic acid fragments is respectively deemed to be 3, 4, 5, 6, 7, 8, 9, or 10 because of the 3 to 1, 4 to 1, 5 to 1, 6 to 1, 7 to 1, 8 to 1, 9 to 1, or 10 to 1 relationship between nucleic acid fragments in the plurality of nucleic acid fragments and the sequence reads.

In some embodiments, on average, each cell-free nucleic acid fragment in the plurality of nucleic acid fragments is represented by 20, 25, 30, 35, 40, 45, 50, or 55 different sequence reads from the nucleic acid sequencing. In such instances, the coverage of the cell-free nucleic acid fragments is respectively deemed to be 20, 25, 30, 35, 40, 45, 50, or 55 because of the 20 to 1, 25 to 1, 30 to 1, 40 to 1, 45 to 1, 50 to 1, or 55 to 1 relationship between nucleic acid fragments in the plurality of nucleic acid fragments and the sequence reads.

In some embodiments, each nucleic acid fragment corresponds to (contains) one respective methylation site. In some such embodiments, each nucleic acid fragment has a single respective methylation state. In some such embodiments, each nucleic acid fragment may have more than a single respective methylation state but only the single respective methylation state is polled and the remaining methylation sites are not evaluated.

In some embodiments, each nucleic acid fragment corresponds to (contains) one or more respective methylation sites. In such embodiments, each nucleic acid fragment has one or more methylation states, where each methylation state corresponds to a respective methylation site. In some embodiments, each nucleic acid fragment includes at least one methylation site, at least two methylation sites, at least five methylation sites, or at least ten methylation sites. In some embodiments, each nucleic acid fragment in the plurality of nucleic acid fragments includes the same number of methylation sites. In some embodiments, each respective nucleic acid fragment in the plurality of nucleic acid fragments includes an independent number of methylation sites which may be the same or different than the number methylation sites in other nucleic acid fragments. In some embodiments, nucleic acid fragments from at least one set of nucleic acid fragments from the plurality of nucleic acid fragments include a different number of methylation sites than the number of methylation sites included in the nucleic acid fragments in a second set of nucleic acid fragments.

The methylation state of a respective nucleic acid fragment in the plurality of nucleic acid fragments, embodied in the sequence of the nucleic acid fragment, represents the methylation state of the cell-free nucleic acid fragment.

In some embodiments, the first cell source of block 202 of FIG. 2A is a first cancer of a common primary site of origin. In some embodiments, the first cancer is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof.

In some embodiments, the first cell source is a tumor of a certain cancer type, or a fraction thereof. In some embodiments, the tumor is an adrenocortical carcinoma, a childhood adrenocortical carcinoma, a tumor of an AIDS-related cancer, kaposi sarcoma, a tumor associated with anal cancer, a tumor associated with an appendix cancer, an astrocytoma, a childhood (brain cancer) tumor, an atypical teratoid/rhabdoid tumor, a central nervous system (brain cancer) tumor, a basal cell carcinoma of the skin, a tumor associated with bile duct cancer, a bladder cancer tumor, a childhood bladder cancer tumor, a bone cancer (e.g., ewing sarcoma and osteosarcoma and malignant fibrous histiocytoma) tissue, a brain tumor, breast cancer tissue, childhood breast cancer tissue, a childhood bronchial tumor, burkitt lymphoma tissue, a carcinoid tumor (gastrointestinal), a childhood carcinoid tumor, a carcinoma of unknown primary, a childhood carcinoma of unknown primary, a childhood cardiac (heart) tumor, a central nervous system (e.g., brain cancer such as childhood atypical teratoid/rhabdoid) tumor, a childhood embryonal tumor, a childhood germ cell tumor, cervical cancer tissue, childhood cervical cancer tissue, cholangiocarcinoma tissue, childhood chordoma tissue, a chronic myeloproliferative neoplasm, a colorectal cancer tumor, a childhood colorectal cancer tumor, childhood craniopharyngioma tissue, a ductal carcinoma in situ (DCIS), a childhood embryonal tumor, endometrial cancer (uterine cancer) tissue, childhood ependymoma tissue, esophageal cancer tissue, childhood esophageal cancer tissue, esthesioneuroblastoma (head and neck cancer) tissue, a childhood extracranial germ cell tumor, an extragonadal germ cell tumor, eye cancer tissue, an intraocular melanoma, a retinoblastoma, fallopian tube cancer tissue, gallbladder cancer tissue, gastric (stomach) cancer tissue, childhood gastric (stomach) cancer tissue, a gastrointestinal carcinoid tumor, a gastrointestinal stromal tumor (GIST), a childhood gastrointestinal stromal tumor, a germ cell tumor (e.g., a childhood central nervous system germ cell tumor, a childhood extracranial germ cell tumor, an extragonadal germ cell tumor, an ovarian germ cell tumor, or testicular cancer tissue), head and neck cancer tissue, a childhood heart tumor, hepatocellular cancer (HCC) tissue, an islet cell tumor (pancreatic neuroendocrine tumors), kidney or renal cell cancer (RCC) tissue, laryngeal cancer tissue, leukemia, liver cancer tissue, lung cancer (non-small cell and small cell) tissue, childhood lung cancer tissue, male breast cancer tissue, a malignant fibrous histiocytoma of bone and osteosarcoma, a melanoma, a childhood melanoma, an intraocular melanoma, a childhood intraocular melanoma, a merkel cell carcinoma, a malignant mesothelioma, a childhood mesothelioma, metastatic cancer tissue, metastatic squamous neck cancer with occult primary tissue, a midline tract carcinoma with NUT gene changes, mouth cancer (head and neck cancer) tissue, multiple endocrine neoplasia syndrome tissue, a multiple myeloma/plasma cell neoplasm, myelodysplastic syndrome tissue, a myelodysplastic/myeloproliferative neoplasm, a chronic myeloproliferative neoplasm, nasal cavity and paranasal sinus cancer tissue, nasopharyngeal cancer (NPC) tissue, neuroblastoma tissue, non-small cell lung cancer tissue, oral cancer tissue, lip and oral cavity cancer and oropharyngeal cancer tissue, osteosarcoma and malignant fibrous histiocytoma of bone tissue, ovarian cancer tissue, childhood ovarian cancer tissue, pancreatic cancer tissue, childhood pancreatic cancer tissue, papillomatosis (childhood laryngeal) tissue, paraganglioma tissue, childhood paraganglioma tissue, paranasal sinus and nasal cavity cancer tissue, parathyroid cancer tissue, penile cancer tissue, pharyngeal cancer tissue, pheochromocytoma tissue, childhood pheochromocytoma tissue, a pituitary tumor, a plasma cell neoplasm/multiple myeloma, a pleuropulmonary blastoma, a primary central nervous system (CNS) lymphoma, primary peritoneal cancer tissue, prostate cancer tissue, rectal cancer tissue, a retinoblastoma, a childhood rhabdomyosarcoma, salivary gland cancer tissue, a sarcoma (e.g., a childhood vascular tumor, osteosarcoma, uterine sarcoma, etc.), Sezary syndrome (lymphoma) tissue, skin cancer tissue, childhood skin cancer tissue, small cell lung cancer tissue, small intestine cancer tissue, a squamous cell carcinoma of the skin, a squamous neck cancer with occult primary, a cutaneous t-cell lymphoma, testicular cancer tissue, childhood testicular cancer tissue, throat cancer (e.g., nasopharyngeal cancer, oropharyngeal cancer, hypopharyngeal cancer) tissue, a thymoma or thymic carcinoma, thyroid cancer tissue, transitional cell cancer of the renal pelvis and ureter tissue, unknown primary carcinoma tissue, ureter or renal pelvis tissue, transitional cell cancer (kidney (renal cell) cancer tissue, urethral cancer tissue, endometrial uterine cancer tissue, uterine sarcoma tissue, vaginal cancer tissue, childhood vaginal cancer tissue, a vascular tumor, vulvar cancer tissue, a Wilms tumor or other childhood kidney tumor.

In some embodiments, the first cell source of block 202 of FIG. 2A is a first cancer. In some such embodiments, the first cancer is a stage of a breast cancer, a stage of a lung cancer, a stage of a prostate cancer, a stage of a colorectal cancer, a stage of a renal cancer, a stage of a uterine cancer, a stage of a pancreatic cancer, a stage of a cancer of the esophagus, a stage of a lymphoma, a stage of a head/neck cancer, a stage of a ovarian cancer, a stage of a hepatobiliary cancer, a stage of a melanoma, a stage of a cervical cancer, a stage of a multiple myeloma, a stage of a leukemia, a stage of a thyroid cancer, a stage of a bladder cancer, or a stage of a gastric cancer.

In some embodiments, the first cell source of block 202 of FIG. 2A is a predetermined stage of a breast cancer, a predetermined stage of a lung cancer, a predetermined stage of a prostate cancer, a predetermined stage of a colorectal cancer, a predetermined stage of a renal cancer, a predetermined stage of a uterine cancer, a predetermined stage of a pancreatic cancer, a predetermined stage of a cancer of the esophagus, a predetermined stage of a lymphoma, a predetermined stage of a head/neck cancer, a predetermined stage of a ovarian cancer, a predetermined stage of a hepatobiliary cancer, a predetermined stage of a melanoma, a predetermined stage of a cervical cancer, a predetermined stage of a multiple myeloma, a predetermined stage of a leukemia, a predetermined stage of a thyroid cancer, a predetermined stage of a bladder cancer, or a predetermined stage of a gastric cancer.

In some embodiments, the first cell source of block 202 of FIG. 2A is from a non-cancerous tissue. In some embodiments, the first cell source is from cells that derive from healthy tissue. In some embodiments, the first cell source is from a healthy tissue such as breast, lung, prostate, colorectal, renal, uterine, pancreatic, esophageal, lymph, ovarian, cervical, epidermal, thyroid, bladder, gastric, or a combination thereof. In some embodiments, the first cell source is a composite healthy source that contains healthy cells from several different healthy tissues (e.g., breast, lung, prostate, colorectal, renal, uterine, pancreatic, esophageal, lymph, ovarian, cervical, epidermal, thyroid, bladder, gastric, or a combination thereof).

In some embodiments, the first cell source is derived from one tissue type. In some embodiments, the first cell source is derived from two or more tissue types. In some embodiments, a tissue type includes one or more cell types (e.g., a combination of healthy, non-cancerous cells and cancerous cells). In some embodiments, a tissue type includes one cell type (e.g., one of either cancerous or healthy, non-cancerous cells).

In some embodiments, the first cell source constitutes one cell type, two cell types, three cell types, four cell types, five cell types, six cell types, seven cell types, eight cell types, nine cell types, ten cell types, or more than ten cell types.

In some embodiments, the first cell source is liver cells. In some such embodiments, the cell source is hepatocytes, hepatic stellate fat storing cells (ITO cells), Kupffer cells, sinusoidal endothelial cells, or any combination thereof.

In some embodiments, the first cell source is stomach cells. In some such embodiments, the first cell source is parietal cells.

In some embodiments, the first cell source is any combination of cell types provided that such cell types originated from a single organ. In some such embodiments this single organ is breast, lung, prostate, colon/rectum, kidney, uterus, pancreas, esophagus, blood, head/neck, ovary, liver, cervix, thyroid, bladder, or stomach. In some embodiments this single organ is healthy. In alternative embodiments this single organ is afflicted with cancer that originated in the single organ. In still further alternative embodiments, this single organ is afflicted with cancer that originated in an organ other than the single organ and metastasized to the single organ.

In some embodiments, the first cell source is any combination of cell types provided that such cell types originated from a predetermined set of organs. In some such embodiments this predetermined set of organs is any two organs in the set breast, lung, prostate, colon/rectum, kidney, uterus, pancreas, esophagus, blood, head/neck, ovary, liver, cervix, thyroid, bladder, and stomach. In some embodiments this predetermined set of organs is healthy. In alternative embodiments this predetermined set of organs is afflicted with cancer that originated in one of the organs in the predetermined set of organs. In still further alternative embodiments, the predetermined set of organs is afflicted with cancer that originated in an organ other than the predetermined set of organs and metastasized to the predetermined set of organs.

In some embodiments, the first cell source is any combination of cell types provided that such cell types originated from a predetermined set of organs. In some such embodiments this predetermined set of organs is any three organs in the set breast, lung, prostate, colon/rectum, kidney, uterus, pancreas, esophagus, blood, head/neck, ovary, liver, cervix, thyroid, bladder, and stomach. In some embodiments this predetermined set of organs is healthy. In alternative embodiments this predetermined set of organs is afflicted with cancer that originated in one of the organs in the predetermined set of organs. In still further alternative embodiments, the predetermined set of organs is afflicted with cancer that originated in an organ other than the predetermined set of organs and metastasized to the predetermined set of organs.

In some embodiments, the first cell source is any combination of cell types provided that such cell types originated from a predetermined set of organs. In some such embodiments this predetermined set of organs is any four organs, five organs, six organs, or seven organs in the set breast, lung, prostate, colon/rectum, kidney, uterus, pancreas, esophagus, blood, head/neck, ovary, liver, cervix, thyroid, bladder, and stomach. In some embodiments this predetermined set of organs is healthy. In alternative embodiments this predetermined set of organs is afflicted with cancer that originated in one of the organs in the predetermined set of organs. In still further alternative embodiments, the predetermined set of organs is afflicted with cancer that originated in an organ other than the predetermined set of organs and metastasized to the predetermined set of organs.

In some specific embodiments, the first cell source is white blood cells. In some such embodiments, the first cell source is neutrophils, eosinophils, basophils, lymphocytes, B lymphocytes, T lymphocytes, cytotoxic T cells, monocytes, or any combination thereof.

In some embodiments, the sequence reads for nucleic acid fragments 128 are pre-processed to correct biases or errors using one or more methods such as normalization, correction of GC biases, and/or correction of biases due to PCR over-amplification.

In some embodiments, the sequence reads for the nucleic acid fragments 126 taken from the biological sample provide a coverage rate of 1× or greater, 2× or greater, 5× or greater, 10× or greater, or 50× or greater for at least three methylation sites, at least five methylation sites, at least ten methylation sites, at least twenty methylation sites, at least thirty methylation sites, at least forty methylation sites, at least fifty methylation sites, at least sixty methylation sites, at least seventy methylation sites, at least eighty methylation sites, at least ninety methylation sites, at least 200 methylation sites, at least 300 methylation sites, at least 400 methylation sites, at least 500 methylation sites or at least 1000 methylation sites from the genome of the subject.

In some embodiments, the subject is human and the first plurality of nucleic acid fragments 128 are obtained through whole genome bisulfite sequencing where a nucleic sample undergoes a bisulfite treatment before the converted nucleic acid molecules are evaluated for sequencing information and methylation status on a genome-wide basis. In some embodiments, the whole genome bisulfite sequencing assay looks for variations in methylation patterns in the genome. See, for example, Example 7. See also, United States Patent Publication No. 20190287652, entitled “Anomalous Fragment Detection and Classification,” filed Mar. 13, 2019, which is hereby incorporated by reference. In some embodiments, enzymatic conversion processes may be used to treat the nucleic acids prior to sequencing, which can be performed in various ways. An example of a bisulfite-free conversion is described in Liu et at. that describe a bisulfite-free and base-resolution sequencing method, TET-assisted pyridine borane sequencing (TAPS), for non-destructive and direct detection of 5-methylcytosine and 5-hydroxymethylcytosine without affecting unmodified cytosines. See, Liu et al., 2019, “Bisulfite-free direct detection of 5-methylcytosine and 5-hydroxymethylcytosine at base resolution,” Nat Biotechnol. 37, pages 424-429, which is hereby incorporated by reference herein in its entirety. In some embodiments, regardless of the specific enzymatic conversion approach, only the methylated cytosines are converted.

In some embodiments, the targeted sequencing is targeted DNA methylation sequencing. The targeted DNA methylation sequencing can be performed in various ways. Different enzymatic treatments and combination with chemical treatment(s) can convert either methylated cytosines or unmethylated cytosines. For example, in some embodiments, the targeted DNA methylation sequencing detects one or more 5-methylcytosine (5mC) and/or 5-hydroxymethylcytosine (5hmC) in the plurality of nucleic acids. As another example, the targeted DNA methylation sequencing may comprise conversion of one or more unmethylated cytosines or one or more methylated cytosines, in the plurality of nucleic acids, to a corresponding one or more uracils. As another example, in some embodiments, the targeted DNA methylation sequencing may comprise conversion of one or more unmethylated cytosines, in the plurality of nucleic acids, to a corresponding one or more uracils, and the DNA methylation sequence reads out the one or more uracils as one or more corresponding thymines. In some embodiments, the targeted DNA methylation sequencing comprises conversion of one or more methylated cytosines, in the plurality of nucleic acids, to one or more corresponding uracils, and the DNA methylation sequence reads out the one or more 5mC or 5hmC as one or more corresponding thymines.

In targeted methylation sequencing process, probes are used to enrich the nucleic acid samples. In some embodiments, probes may be designed such that they bind to sequences after cytosines in methylated CpG sites or un-methylated CpG sites are converted (e.g., in a chemical or enzymatic conversion process). In embodiments in which methylation sequencing is used, sequences of the probes may not be complementary to the corresponding genomic sequence but rather to the sequences of the converted DNA fragments.

Block 208. The method proceeds by individually assigning a first score 132 to each respective nucleic acid fragment 128 in the first plurality of nucleic acid fragments, thereby obtaining a plurality of first scores. In some embodiments, each respective first score represents a likelihood that the corresponding nucleic acid fragment originated from the first cell source. In some embodiments, each respective first score represents a binary indicator (e.g., positive or negative) indicating whether the corresponding nucleic acid fragment was obtained from the first cell source. In some embodiments, the binary indicator indicates that the corresponding nucleic acid fragment is derived from the first cell source when the first score is over an indicator predefined threshold. In some embodiments, the indicator predefined threshold is at least fifty percent, at least sixty percent, at least seventy-five percent, at least eighty-five percent, at least ninety percent, at least ninety-five percent, or at least ninety-eight percent.

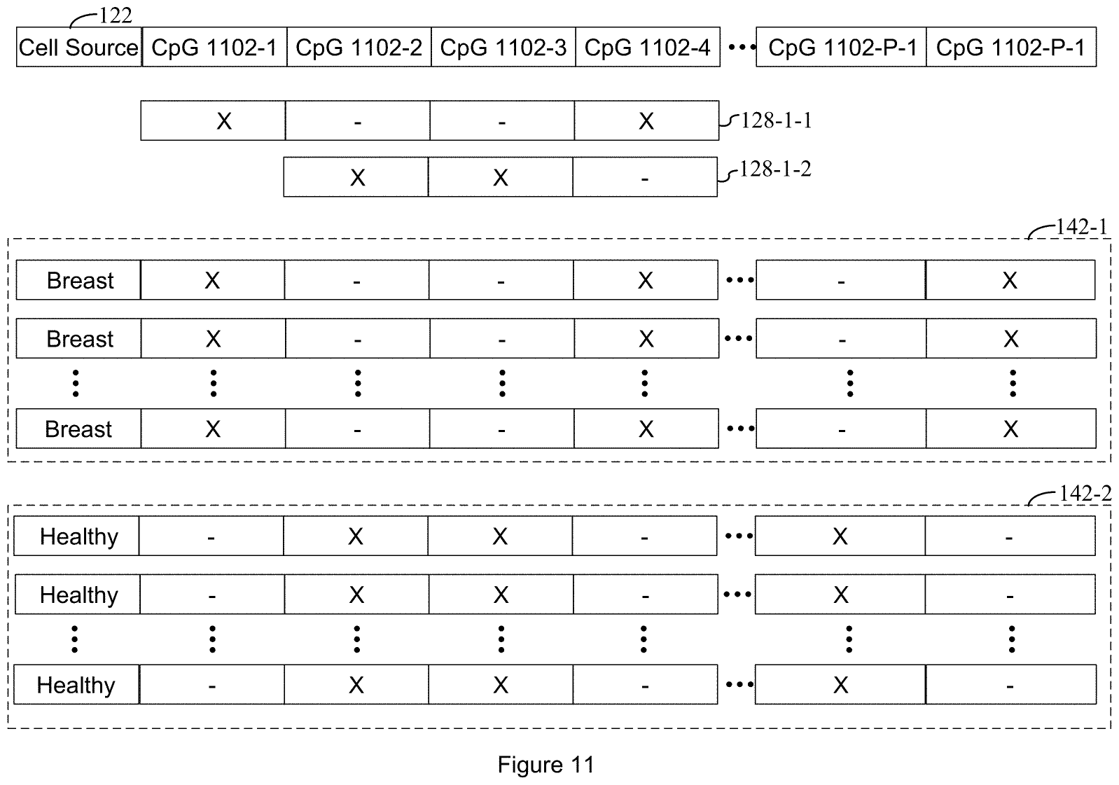

In some embodiments, the individually assigning comprises i) comparing a methylation state of the respective nucleic acid fragment against a first canonical set of methylation state vectors and against a second canonical set of methylation state vectors, or ii) presenting the methylation state of the respective nucleic acid fragment to a first classifier trained at least in part on the first canonical set of methylation state vectors and the second canonical set of methylation state vectors. FIG. 11 illustrates a non-limiting example in which the first canonical set of methylation state vectors is derived from reference subjects having breast cancer (142-1 in FIG. 11), and the second canonical set of methylation state vectors is derived from biological samples of reference subjects that are healthy (142-2 in FIG. 11). In FIG. 11, the methylation state of two nucleic acid fragments, 128-1-1 and 128-1-2 from the biological sample of a test subject are assigned scores by comparing a methylation state of nucleic acid fragments 128-1-1 and 128-1-2 against the canonical set of methylation state vectors for breast cancer 142-1 and against the canonical set of methylation state vectors representative of healthy tissue 142-2.

In some embodiments, the individually assigning comprises comparing a methylation state of the respective nucleic acid fragment against a first canonical set of methylation vectors. In such embodiments, no second canonical set of methylation state vectors is required.

It is seen in FIG. 11 that, of the four methylation sites in nucleic acid fragment 128-1-1, they match the pattern of methylation found in the canonical set of methylation state vectors for breast cancer 142-1 rather than the canonical set of methylation state vectors representative of healthy tissue 142-2. Therefore, nucleic acid fragment 128-1-1 is assigned a first score 132 that represents a strong likelihood that the nucleic acid fragment originated from breast cancer.

It is further seen in FIG. 11 that, of the three methylation sites in nucleic acid fragment 128-1-2, they match the pattern of methylation found in the canonical set of methylation state vectors for healthy tissue/cells 142-2 rather than the canonical set of methylation state vectors representative of breast cancer 142-1. Therefore, nucleic acid fragment 128-1-1 is assigned first score 132 that represents a very low likelihood that the nucleic acid fragment originated from breast cancer.

FIG. 11 illustrates some pertinent points. First, the present application leverages the observation that the methylation pattern of particular regions of the genome, for any given cell type (e.g., a particular cancer type) is quite stable, meaning that circulating nucleic acid fragments of such portions of the genome from such cell types have a stable methylation pattern, meaning that methylation sites in such regions are consistently methylated or not methylated in the same manner. As such, such regions of the genome are informative for discerning that nucleic acid fragments mapping encompassing such regions and that have the same hallmark methylation pattern, in fact, originate from such cell sources. This is seen in canonical set 142-1, where the methylation pattern, nominally “X” for methylated and “−” for unmethylated, is the same at each respective methylation (CpG) site across the canonical breast cancer set. This is also seen in canonical set 142-2, where the methylation pattern, nominally “X” for methylated and “−” for unmethylated, is the same at each respective methylation (CpG) site across the canonical healthy set.

Of course, there can be variation in the methylation pattern even in the informative regions of the genome, for any given cell type. This may arise due to additional factors such as patient age, confounding disease conditions, and other conditions. As such, contrary to what is illustrated in FIG. 11, in some embodiments, the methylation pattern of each reference subject in the canonical set 142 may not be identical.

In some embodiments, the first score 132 a nucleic acid fragment 128 obtained is a binary score for the first cell source, meaning that the nucleic acid fragment 128 either has been deemed to originate from the first cell source or not. This is exemplified in FIG. 11.

However, in some embodiments, the first score 132 that a nucleic acid fragment 128 obtains is a likelihood for the first cell source, meaning that the nucleic acid fragment 128 is assigned a likelihood that it originates from the first cell source. In some embodiments, this likelihood falls into a range of zero (meaning it did not originate from the first cell source) to 1 (meaning that the probability that the nucleic acid fragment, based on the methylation state vector matching, originated from the first cell source is one hundred percent). Non-binary scoring is not illustrated in FIG. 11 because illustrated nucleic acid fragments 128-1-1 and 128-1-2 each exactly match the methylation state consensus sequence of a canonical set of methylation state vectors. However, the present disclosure encompasses embodiments in which either (i) the methylation state vector across the canonical set of methylation state vectors is not identical and or (ii) the nucleic acid fragment does not exactly match the methylation state vectors of any of the canonical sets of methylation state vectors that the nucleic acid fragment is compared to.

Another point that is illustrated in FIG. 11 is that a nucleic acid fragment can have more than one methylation state. That is, the nucleic acid fragment can have multiple methylation sites, each with a methylation state (e.g., either methylated or not methylated). This is advantageously used to score the nucleic acid fragment since it is clear that the entire nucleic acid fragment had to be derived from the same cell source. Thus, the methylation state vector of the nucleic acid fragment, having more than one element, is used to score the entire nucleic acid fragment, thereby compounding and concurrently leveraging the informative contribution of more than methylation site in the nucleic acid fragment to improve the confidence of the score of the nucleic acid fragment with respect to a cell source.

Yet another point to disclose with respect to FIG. 11 is that the present disclosure is not limited to assigning a single score to a nucleic acid fragment for a single cell source. Indeed, in the case of FIG. 11, for the sake of bookkeeping, a second score can be assigned to each nucleic acid fragment, where the first score still represents the likelihood that the nucleic acid fragment originated from the first cell source (breast cancer in FIG. 11) and the second score represents the likelihood that the nucleic acid fragment originated from a second cell source (healthy cells). In the case where only two cell sources are considered, the second score is not strictly necessary since it can be inferred from the first score. However, in instances where there are more than two canonical sets of nucleic acid fragments and the score assigned is a probability, more than one score may be needed. For example, consider the case where the methylation state of each nucleic acid fragment is compared to three canonical sets of methylation state vectors and, from this comparison, the nucleic acid fragment is determined to have a seventy percent chance of arising from the cell source associated with the first canonical set of methylation state vectors, a twenty percent chance of arising from the cell source associated with the second canonical set of methylation state vectors, and a ten percent chance of arising from the cell source associated with the third canonical set of methylation state vectors. In such an instance the nucleic acid fragment can be assigned a corresponding first score of seventy percent, a corresponding second score of twenty percent, and a corresponding third score of ten percent to reflect these likelihoods. As such, in some embodiments, a respective nucleic acid fragment is assigned two, three, four, five, six, seven, eight, nine or 10 or more first scores, where each such score is an indication of a probability (or other form of metric) that the respective nucleic acid fragment originates from a corresponding cell sources in a plurality of cell sources.

In some embodiments, the comparing the respective nucleic acid fragment against any other canonical set of methylation state vectors other than the first the canonical set of methylation state vectors (such as the second canonical set of methylation state vectors) is optional.

Another point that is illustrated in FIG. 11 is that each nucleic acid fragment is mapped to a reference genome and thus it is understood which part of the canonical methylation state vectors the nucleic acid fragment is to be scored against. In typical embodiments, the canonical methylation state vectors are across the entire genome, or at least the portions of the genome that are informative, with respect to methylation state, for the cell source represented by the set of canonical methylation state vectors that the respective methylation state vectors are in. As such, in typical embodiments, the score assigned to a nucleic acid fragment is only based on all or a portion of the methylation sites that are in the nucleic acid fragment. In some embodiments, the score assigned to a nucleic acid fragment is only based on all the methylation sites that are in the nucleic acid fragment. In some embodiments, the score assigned to a nucleic acid fragment is only based on a single methylation site in the nucleic acid fragment.

Another point to disclose with respect to FIG. 11 is that, in some embodiments, the comparison of the methylation state of the respective nucleic acid fragment against a first canonical set of methylation state vectors compares the methylation state of the respective nucleic acid fragment against that portion of a methylation pattern consensus vector of the first canonical set of methylation state vectors that the respective nucleic acid fragment maps onto. Correspondingly, the comparison of the methylation state of the respective nucleic acid fragment against a second canonical set of methylation state vectors compares the methylation state of the respective nucleic acid fragment against that portion of a methylation pattern consensus vector of the second canonical set of methylation state vectors that the respective nucleic acid fragment maps onto.

In alternative embodiments, the comparison of the methylation state of the respective nucleic acid fragment against a first canonical set of methylation state vectors compares the methylation state of the respective nucleic acid fragment against the methylation pattern of each methylation state vector in the canonical set of methylation state vectors that the respective nucleic acid fragment maps onto. Correspondingly, the comparison of the methylation state of the respective nucleic acid fragment against a second canonical set of methylation state vectors compares the methylation state of the respective nucleic acid fragment against the methylation pattern of each methylation state vector in the second canonical set of methylation state vectors that the respective nucleic acid fragment maps onto.

In some embodiments, rather than comparing a methylation state of the respective nucleic acid fragment against a first canonical set of methylation state vectors and against a second canonical set of methylation state vectors representative of a source other than the first cell source as illustrated in FIG. 11, the label information (cell source 122) together with each methylation state vector in the first and second set of methylation state vectors is used to train a first classifier and the methylation state of the respective nucleic acid fragment of the test subject is applied to this trained first classifier trained to determine the score for cell source for the nucleic acid fragment.

In some embodiments, each canonical methylation state vector in the first canonical set of methylation state vectors is derived from a tissue sample or cell-free nucleic acid sample of a corresponding reference subject in a first plurality of reference subjects corresponding to the first cell source. In some embodiments, each canonical methylation state vector in the first canonical set of methylation state vectors represents the methylation state across the genome of the corresponding reference subject in the first plurality of reference subjects.

In some embodiments, a canonical methylation state vector in the first canonical set of methylation state vectors is derived from a tumor sample of the corresponding reference subject.

In some embodiments, for example, if the biological sample for a respective reference subject is derived from cell-free nucleic acids, it is advantageous that the cell-free nucleic acids exhibit an appreciable tumor (or cell source) fraction. In some embodiments, a canonical methylation state vector in the first set of canonical methylation state vectors is derived from cell-free nucleic acids of a corresponding reference subject in which the tumor fraction, with respect to the first cell source, for the corresponding reference subject is at least two percent, at least five percent, at least ten percent, at least fifteen percent, at least twenty percent, at least twenty-five percent, at least fifty percent, at least seventy-five percent, at least ninety percent, at least ninety-five percent, or at least ninety-eight percent.

In some embodiments, each canonical methylation state vector in the first canonical set of methylation state vectors represents the methylation state across a subset of the genome of the corresponding reference subject, where a methylation state of the subset of the genome is representative of causative biology underlying the first cell source.

In some embodiments, each canonical methylation state vector in the second canonical set of methylation state vectors is derived from a tissue sample or cell-free nucleic acid sample of a corresponding reference subject in a second plurality of reference subjects corresponding to a second cell source.

In some embodiments, the first canonical set of methylation state vectors is a single consensus methylation state vector of the genome of the species formed from a methylation state of nucleic acids in the tissue sample or cell-free nucleic acid sample across the first plurality of reference subjects. In some embodiments, the second canonical set of methylation state vectors is a single consensus methylation state vector of the genome of the species formed from a methylation state of nucleic acids in the tissue sample or cell-free nucleic acid sample across the second plurality of reference subjects.

In some embodiments, the first canonical set of methylation state vectors includes a different consensus methylation state vector of the genome of the species for each respective reference subject in the first plurality of reference subjects formed from a methylation state of nucleic acids in the tissue sample or cell-free nucleic acid sample of the respective reference subject. In some embodiments, the second canonical set of methylation state vectors includes a different consensus methylation state vector of the genome of the species for each respective reference subject in the second plurality of reference subjects formed from a methylation state of nucleic acids in the tissue sample or cell-free nucleic acid sample of the respective reference subject

In some embodiments, the second cell source is a healthy cancer-free state. In some such embodiments, this healthy cancer-free state is formed from cell-free nucleic acids from liquid biopsies obtained from healthy subjects. In alternative embodiments, this healthy cancer-free state is formed from nucleic acids from solid biopsies obtained from one or more organs of healthy subjects. In some such embodiments, the one or more organs include biopsies from any number for different tissues (e.g., breast, lung, prostate, rectum, uterus, pancreas, esophagus, head/neck, ovaries, cervix, thyroid, bladder or a combination thereof).

In some embodiments, the second cell source is a second cancer of a common primary site of origin. In some embodiments, the second cancer is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof.

In some embodiments, the first cell source of block 202 of FIG. 2A is a first cancer of a common primary site of origin. In some embodiments, the first cancer is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof. In some such embodiments, the second cell source fulfills the twin requirements of being both (i) other than the cells of the first cell source and (ii) being breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof. In some alternative embodiments, the second cell source is all cells that are not of the first cell source. In some alternative embodiments, the second cell source is all cancer cells that are not of the first cell source. In some alternative embodiments, the second cell source is all healthy cells.

In some embodiments, the first cell source is a tumor of a certain cancer type, or a fraction thereof. In some embodiments, the tumor is an adrenocortical carcinoma, a childhood adrenocortical carcinoma, a tumor of an AIDS-related cancer, kaposi sarcoma, a tumor associated with anal cancer, a tumor associated with an appendix cancer, an astrocytoma, a childhood (brain cancer) tumor, an atypical teratoid/rhabdoid tumor, a central nervous system (brain cancer) tumor, a basal cell carcinoma of the skin, a tumor associated with bile duct cancer, a bladder cancer tumor, a childhood bladder cancer tumor, a bone cancer (e.g., ewing sarcoma and osteosarcoma and malignant fibrous histiocytoma) tissue, a brain tumor, breast cancer tissue, childhood breast cancer tissue, a childhood bronchial tumor, burkitt lymphoma tissue, a carcinoid tumor (gastrointestinal), a childhood carcinoid tumor, a carcinoma of unknown primary, a childhood carcinoma of unknown primary, a childhood cardiac (heart) tumor, a central nervous system (e.g., brain cancer such as childhood atypical teratoid/rhabdoid) tumor, a childhood embryonal tumor, a childhood germ cell tumor, cervical cancer tissue, childhood cervical cancer tissue, cholangiocarcinoma tissue, childhood chordoma tissue, a chronic myeloproliferative neoplasm, a colorectal cancer tumor, a childhood colorectal cancer tumor, childhood craniopharyngioma tissue, a ductal carcinoma in situ (DCIS), a childhood embryonal tumor, endometrial cancer (uterine cancer) tissue, childhood ependymoma tissue, esophageal cancer tissue, childhood esophageal cancer tissue, esthesioneuroblastoma (head and neck cancer) tissue, a childhood extracranial germ cell tumor, an extragonadal germ cell tumor, eye cancer tissue, an intraocular melanoma, a retinoblastoma, fallopian tube cancer tissue, gallbladder cancer tissue, gastric (stomach) cancer tissue, childhood gastric (stomach) cancer tissue, a gastrointestinal carcinoid tumor, a gastrointestinal stromal tumor (GIST), a childhood gastrointestinal stromal tumor, a germ cell tumor (e.g., a childhood central nervous system germ cell tumor, a childhood extracranial germ cell tumor, an extragonadal germ cell tumor, an ovarian germ cell tumor, or testicular cancer tissue), head and neck cancer tissue, a childhood heart tumor, hepatocellular cancer (HCC) tissue, an islet cell tumor (pancreatic neuroendocrine tumors), kidney or renal cell cancer (RCC) tissue, laryngeal cancer tissue, leukemia, liver cancer tissue, lung cancer (non-small cell and small cell) tissue, childhood lung cancer tissue, male breast cancer tissue, a malignant fibrous histiocytoma of bone and osteosarcoma, a melanoma, a childhood melanoma, an intraocular melanoma, a childhood intraocular melanoma, a merkel cell carcinoma, a malignant mesothelioma, a childhood mesothelioma, metastatic cancer tissue, metastatic squamous neck cancer with occult primary tissue, a midline tract carcinoma with NUT gene changes, mouth cancer (head and neck cancer) tissue, multiple endocrine neoplasia syndrome tissue, a multiple myeloma/plasma cell neoplasm, myelodysplastic syndrome tissue, a myelodysplastic/myeloproliferative neoplasm, a chronic myeloproliferative neoplasm, nasal cavity and paranasal sinus cancer tissue, nasopharyngeal cancer (NPC) tissue, neuroblastoma tissue, non-small cell lung cancer tissue, oral cancer tissue, lip and oral cavity cancer and oropharyngeal cancer tissue, osteosarcoma and malignant fibrous histiocytoma of bone tissue, ovarian cancer tissue, childhood ovarian cancer tissue, pancreatic cancer tissue, childhood pancreatic cancer tissue, papillomatosis (childhood laryngeal) tissue, paraganglioma tissue, childhood paraganglioma tissue, paranasal sinus and nasal cavity cancer tissue, parathyroid cancer tissue, penile cancer tissue, pharyngeal cancer tissue, pheochromocytoma tissue, childhood pheochromocytoma tissue, a pituitary tumor, a plasma cell neoplasm/multiple myeloma, a pleuropulmonary blastoma, a primary central nervous system (CNS) lymphoma, primary peritoneal cancer tissue, prostate cancer tissue, rectal cancer tissue, a retinoblastoma, a childhood rhabdomyosarcoma, salivary gland cancer tissue, a sarcoma (e.g., a childhood vascular tumor, osteosarcoma, uterine sarcoma, etc.), Sézary syndrome (lymphoma) tissue, skin cancer tissue, childhood skin cancer tissue, small cell lung cancer tissue, small intestine cancer tissue, a squamous cell carcinoma of the skin, a squamous neck cancer with occult primary, a cutaneous t-cell lymphoma, testicular cancer tissue, childhood testicular cancer tissue, throat cancer (e.g., nasopharyngeal cancer, oropharyngeal cancer, hypopharyngeal cancer) tissue, a thymoma or thymic carcinoma, thyroid cancer tissue, transitional cell cancer of the renal pelvis and ureter tissue, unknown primary carcinoma tissue, ureter or renal pelvis tissue, transitional cell cancer (kidney (renal cell) cancer tissue, urethral cancer tissue, endometrial uterine cancer tissue, uterine sarcoma tissue, vaginal cancer tissue, childhood vaginal cancer tissue, a vascular tumor, vulvar cancer tissue, a Wilms tumor or other childhood kidney tumor. In some such embodiments, the second cell source fulfills the twin requirements of being both (i) other than the first cell source and (ii) being a tumor of a certain cancer type, or a fraction thereof, where the tumor is an adrenocortical carcinoma, a childhood adrenocortical carcinoma, a tumor of an AIDS-related cancer, kaposi sarcoma, a tumor associated with anal cancer, a tumor associated with an appendix cancer, an astrocytoma, a childhood (brain cancer) tumor, an atypical teratoid/rhabdoid tumor, a central nervous system (brain cancer) tumor, a basal cell carcinoma of the skin, a tumor associated with bile duct cancer, a bladder cancer tumor, a childhood bladder cancer tumor, a bone cancer (e.g., ewing sarcoma and osteosarcoma and malignant fibrous histiocytoma) tissue, a brain tumor, breast cancer tissue, childhood breast cancer tissue, a childhood bronchial tumor, burkitt lymphoma tissue, a carcinoid tumor (gastrointestinal), a childhood carcinoid tumor, a carcinoma of unknown primary, a childhood carcinoma of unknown primary, a childhood cardiac (heart) tumor, a central nervous system (e.g., brain cancer such as childhood atypical teratoid/rhabdoid) tumor, a childhood embryonal tumor, a childhood germ cell tumor, cervical cancer tissue, childhood cervical cancer tissue, cholangiocarcinoma tissue, childhood chordoma tissue, a chronic myeloproliferative neoplasm, a colorectal cancer tumor, a childhood colorectal cancer tumor, childhood craniopharyngioma tissue, a ductal carcinoma in situ (DCIS), a childhood embryonal tumor, endometrial cancer (uterine cancer) tissue, childhood ependymoma tissue, esophageal cancer tissue, childhood esophageal cancer tissue, esthesioneuroblastoma (head and neck cancer) tissue, a childhood extracranial germ cell tumor, an extragonadal germ cell tumor, eye cancer tissue, an intraocular melanoma, a retinoblastoma, fallopian tube cancer tissue, gallbladder cancer tissue, gastric (stomach) cancer tissue, childhood gastric (stomach) cancer tissue, a gastrointestinal carcinoid tumor, a gastrointestinal stromal tumor (GIST), a childhood gastrointestinal stromal tumor, a germ cell tumor (e.g., a childhood central nervous system germ cell tumor, a childhood extracranial germ cell tumor, an extragonadal germ cell tumor, an ovarian germ cell tumor, or testicular cancer tissue), head and neck cancer tissue, a childhood heart tumor, hepatocellular cancer (HCC) tissue, an islet cell tumor (pancreatic neuroendocrine tumors), kidney or renal cell cancer (RCC) tissue, laryngeal cancer tissue, leukemia, liver cancer tissue, lung cancer (non-small cell and small cell) tissue, childhood lung cancer tissue, male breast cancer tissue, a malignant fibrous histiocytoma of bone and osteosarcoma, a melanoma, a childhood melanoma, an intraocular melanoma, a childhood intraocular melanoma, a merkel cell carcinoma, a malignant mesothelioma, a childhood mesothelioma, metastatic cancer tissue, metastatic squamous neck cancer with occult primary tissue, a midline tract carcinoma with NUT gene changes, mouth cancer (head and neck cancer) tissue, multiple endocrine neoplasia syndrome tissue, a multiple myeloma/plasma cell neoplasm, myelodysplastic syndrome tissue, a myelodysplastic/myeloproliferative neoplasm, a chronic myeloproliferative neoplasm, nasal cavity and paranasal sinus cancer tissue, nasopharyngeal cancer (NPC) tissue, neuroblastoma tissue, non-small cell lung cancer tissue, oral cancer tissue, lip and oral cavity cancer and oropharyngeal cancer tissue, osteosarcoma and malignant fibrous histiocytoma of bone tissue, ovarian cancer tissue, childhood ovarian cancer tissue, pancreatic cancer tissue, childhood pancreatic cancer tissue, papillomatosis (childhood laryngeal) tissue, paraganglioma tissue, childhood paraganglioma tissue, paranasal sinus and nasal cavity cancer tissue, parathyroid cancer tissue, penile cancer tissue, pharyngeal cancer tissue, pheochromocytoma tissue, childhood pheochromocytoma tissue, a pituitary tumor, a plasma cell neoplasm/multiple myeloma, a pleuropulmonary blastoma, a primary central nervous system (CNS) lymphoma, primary peritoneal cancer tissue, prostate cancer tissue, rectal cancer tissue, a retinoblastoma, a childhood rhabdomyosarcoma, salivary gland cancer tissue, a sarcoma (e.g., a childhood vascular tumor, osteosarcoma, uterine sarcoma, etc.), Sezary syndrome (lymphoma) tissue, skin cancer tissue, childhood skin cancer tissue, small cell lung cancer tissue, small intestine cancer tissue, a squamous cell carcinoma of the skin, a squamous neck cancer with occult primary, a cutaneous t-cell lymphoma, testicular cancer tissue, childhood testicular cancer tissue, throat cancer (e.g., nasopharyngeal cancer, oropharyngeal cancer, hypopharyngeal cancer) tissue, a thymoma or thymic carcinoma, thyroid cancer tissue, transitional cell cancer of the renal pelvis and ureter tissue, unknown primary carcinoma tissue, ureter or renal pelvis tissue, transitional cell cancer (kidney (renal cell) cancer tissue, urethral cancer tissue, endometrial uterine cancer tissue, uterine sarcoma tissue, vaginal cancer tissue, childhood vaginal cancer tissue, a vascular tumor, vulvar cancer tissue, a Wilms tumor or other childhood kidney tumor. In some alternative embodiments, the second cell source is cells of all tumor types that do not correspond to the first cell source. In some alternative embodiments, the second cell source is all healthy cells.

In some embodiments, the first cell source of block 202 of FIG. 2A is a first cancer. In some such embodiments, the first cancer is a stage of a breast cancer, a stage of a lung cancer, a stage of a prostate cancer, a stage of a colorectal cancer, a stage of a renal cancer, a stage of a uterine cancer, a stage of a pancreatic cancer, a stage of a cancer of the esophagus, a stage of a lymphoma, a stage of a head/neck cancer, a stage of a ovarian cancer, a stage of a hepatobiliary cancer, a stage of a melanoma, a stage of a cervical cancer, a stage of a multiple myeloma, a stage of a leukemia, a stage of a thyroid cancer, a stage of a bladder cancer, or a stage of a gastric cancer. In some embodiments, the second cell source is a different cancer than that associated with the first cell source. For instance, in some embodiments the first cell source is cells corresponding to breast cancer whereas the second cell source is cells corresponding to stomach cancer. In some alternative embodiments, the second cell source corresponds to all cancers other than the cancer associated with the first cell source. For instance, in some embodiments the first cell source is cells corresponding to breast cancer whereas the second cell source is cells corresponding to all other forms of cancer. In some alternative embodiments, the second cell source is all healthy cells.

In some embodiments, the first cell source of block 202 of FIG. 2A is a predetermined stage of a breast cancer, a predetermined stage of a lung cancer, a predetermined stage of a prostate cancer, a predetermined stage of a colorectal cancer, a predetermined stage of a renal cancer, a predetermined stage of a uterine cancer, a predetermined stage of a pancreatic cancer, a predetermined stage of a cancer of the esophagus, a predetermined stage of a lymphoma, a predetermined stage of a head/neck cancer, a predetermined stage of a ovarian cancer, a predetermined stage of a hepatobiliary cancer, a predetermined stage of a melanoma, a predetermined stage of a cervical cancer, a predetermined stage of a multiple myeloma, a predetermined stage of a leukemia, a predetermined stage of a thyroid cancer, a predetermined stage of a bladder cancer, or a predetermined stage of a gastric cancer. In some embodiments, the second cell source is a different stage of the same cancer associated with the first cell source. For instance, in some embodiments the first cell source is cells corresponding to stage II breast cancer whereas the second cell source is cells corresponding to stage III breast cancer. In some alternative embodiments, the second cell source is the stages of the same cancer associated with the first cell source, other than the specific stage of cancer associated with the first cell source. For instance, in some embodiments the first cell source is cells corresponding to stage I breast cancer whereas the second cell source is cells corresponding to stages, II, III and IV breast cancer. In some embodiments, the second cell source is a stage of a different cancer than that associated with the first cell source. For instance, in some embodiments the first cell source is cells corresponding to stage II breast cancer whereas the second cell source is cells corresponding to stage II stomach cancer. In some alternative embodiments, the second cell source is all healthy cells.

In some embodiments, the first cell source is derived from a first single tissue type. In some such embodiments, the second cell source is derived from a second single tissue type other than that of the first cell type. In alternative embodiments, the second cell source is derived from all tissue types other than that of the first cell type.

In some embodiments, the first cell source is derived from two or more tissue types. In some such embodiments, the second cell source is derived from two or more tissue types other than those of the first cell type. In alternative embodiments, the second cell source is derived from all tissue types other than those of the first cell type.

In some embodiments, the first cell source constitutes one cell type, two cell types, three cell types, four cell types, five cell types, six cell types, seven cell types, eight cell types, nine cell types, ten cell types, or more than ten cell types. In some such embodiments, the second cell source is derived from one cell type, two cell types, three cell types, four cell types, five cell types, six cell types, seven cell types, eight cell types, nine cell types, ten cell types, or more than ten cell types other than those of the first cell type.

In some embodiments, the first cell source is one or more types of human cells. In some such embodiments, the first cell source is adaptive NK cells, adipocytes, alveolar cells, Alzheimer type II astrocytes, amacrine cells, ameloblasts, astrocytes, B cells, basophils, basophil activation cells, basophilia cells, Betz cells, bistratified cells, Boettcher cells, cardiac muscle cells, CD4+ T cells, cementoblasts, cerebellar granule cells, cholangiocytes, cholecystocytes, chromaffin cells, cigar cells, club cells, orticotropic cells, cytotoxic T cells, dendritic cells, enterochromaffin cells, enterochromaffin-like cells, eosinophils, extraglomerular mesangial cells, faggot cells, fat pad cells, gastric chief cells, goblet cells, gonadotropic cells, hepatic stellate cells, hepatocytes, hypersegmented neutrophils, intraglomerular mesangial cells, juxtaglomerular cells, keratinocytes, kidney proximal tubule brush border cells, Kupffer cells, lactotropic cells, Leydig cells, macrophages, macula densa cells, mast cells, megakaryocytes, melanocytes, microfold cells, monocytes, natural killer cells, natural killer T cells, glitter cells, neutrophils, osteoblasts, osteoclasts, osteocytes, oxyphil cells (parathyroid), paneth cells, parafollicular cells, parasol cells, parathyroid chief cells, parietal cells, parvocellular neurosecretory cells, peg cells, pericytes, peritubular myoid cells, platelets, podocytes, regulatory T cell, reticulocytes, retina bipolar cells retina horizontal cells, retinal ganglion cells, retinal precursor cells, sentinel cells, sertoli cells, somatomammotrophic cells, somatotropic cells, stellate cells, sustentacular cells, T cells, T helper cells, telocytes, tendon cells, thyrotropic cells, transitional B cells, trichocytes (human), tuft cells, unipolar brush cells, white blood cells, zellballens, or any combination thereof. In some such embodiments, such cells of the first cell source are healthy. In alternative embodiments such cells of the first cell source are afflicted with cancer. In some such embodiments, the second cell source is derived from a cell type other than that of the first cell type. In alternative embodiments, the second cell source is derived from all cell types other than those of the first cell type.

In some embodiments, the first cell source is any combination of cell types provided that such cell types originated from a single first organ type. In some such embodiments this single first organ type is breast, lung, prostate, colon/rectum, kidney, uterus, pancreas, esophagus, blood, head/neck, ovary, liver, cervix, thyroid, bladder, or stomach. In some such embodiments, the second cell source is any combination of cell types provided that such cell types originated from a single second organ type other than the single first organ type. In some such embodiments this single second organ type is breast, lung, prostate, colon/rectum, kidney, uterus, pancreas, esophagus, blood, head/neck, ovary, liver, cervix, thyroid, bladder, or stomach. In some such embodiments, the second cell source is any combination of cell types provided that such cell types originated from any organ type other than the single first organ type. In some such embodiments the cells of the first cell type are healthy and at least some of the cells of the second cell type are cancerous. In alternative embodiments at least some of the first cell type are cancerous and the cells of the second cell type are healthy.

In some embodiments, the first plurality of reference subjects (whose methylation patterns populate the first canonical set of methylation state vectors) comprises at least ten reference subjects, and the second plurality of reference subjects (whose methylation patterns populate the second canonical set of methylation state vectors) comprises at least ten reference subjects. In some embodiments, the first plurality of reference subjects comprises at least one hundred reference subjects, and the second plurality of reference subjects comprises at least one hundred reference subjects. In some embodiments, the first plurality of reference subjects includes more or less reference subjects than the second plurality of reference subjects. In some embodiments, the first plurality of reference subjects comprises at least 10 reference subjects, at least 25 reference subjects, at least 50 reference subjects, at least 75 reference subjects, at least 100 reference subjects, at least 200 reference subjects, or at least 500 reference subjects.

In some embodiments, the first classifier, described above that is used in some embodiments as an alternative to comparing the methylation state of respective nucleic acid fragments against the first and second canonical sets of methylation state vectors, is based on a multinomial logistic regression algorithm. See for example, Agresti, An Introduction to Categorical Data Analysis, 1996, John Wiley & Sons, Inc., New York, Chapter 8; and Hastie et al., 2001, The Elements of Statistical Learning, Springer-Verlag, New York, each of which are hereby incorporated by reference.

In some embodiments, the first classifier is based on a neural network algorithm. See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. See also, U.S. patent application Ser. No. 16/428,575, entitled “Convolutional Neural Network Systems and Methods for Data Classification,” filed May 31, 2019, which is hereby incorporated by reference, for its disclosure of convolutional neural networks that can be used for classifying methylation patterns in accordance with the present disclosure.

In some embodiments, the first classifier is a support vector machine algorithm. SVMs are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety.

In some embodiments, the first classifier is a Naive Bayes algorithm, such as the tool developed by Rosen et al. to deal with metagenomic reads (See, Bioinformatics 27(1):127-129, 2011). In some embodiments, the classifier is a nearest neighbor algorithm, such as the non-parametric methods described by Kamvar et al., 2015, Front Genetics 6:208 doi: 10.3389/fgene.2015.00208). In some embodiments, the classifier is a mixture model, such as that described in McLachlan et al., 2002, Bioinformatics 18(3):413-422. In some embodiments, in particular those embodiments including a temporal component, the classifier is a hidden Markov model such as described by Schliep et al., 2003, Bioinformatics 19(1):i255-i263.

In some embodiments, the first classifier is a nearest neighbor algorithm, such as the non-parametric methods described by Kamvar et al., Front Genetics 6:208 doi: 10.3389/fgene.2015.00208, 2015). In some embodiments, the first classifier is a mixture model, such as that described in McLachlan et al., Bioinformatics 18(3):413-422, 2002. In some embodiments, in particular those embodiments including a temporal component, the first classifier is a hidden Markov model such as described by Schliep et al., Bioinformatics 19(1):i255-i263, 2003.

Block 220. The method continues by transforming the plurality of first scores into a first plurality of counts. In some embodiments, each count in the first plurality of counts is for a methylation site in a first predetermined set of methylation sites in the genome of a reference sequence of the species. In some embodiments, the first predetermined set of methylation sites is associated with the first cell source.

In some embodiments, the first predetermined set of methylation sites comprises a subset of the genome of the given species. In some embodiments, the first predetermined set of methylation sites comprises fifty methylation sites in the genome of the species. In some embodiments, the first predetermined set of methylation sites comprises one hundred methylation sites in the genome of the species. In some embodiments, the first predetermined set of methylation sites comprises five hundred methylation sites in the genome of the species. In some embodiments, the first predetermined set of methylation sites comprises at least 5 methylation sites, at least 10 methylation sites, at least 15 methylation sites, at least 20 methylation sites, at least 25 methylation sites, at least 50 methylation sites, at least 100 methylation sites, at least 200 methylation sites, at least 500 methylation sites, at least 1000 methylation sites, at least 5000 methylation sites, at least 10,000 methylation sites, or at least 20,000 methylation sites.

In some embodiments, the transforming the plurality of first scores into a first plurality of counts further comprises, for each respective methylation site in the first predetermined set of methylation sites: (a) determining a first number of nucleic acid fragments in the first plurality of nucleic acid fragments that (i) map to the respective methylation site and (ii) have a first score satisfying a threshold value; (b) determining a second number of nucleic acid fragments in the plurality of nucleic acid fragments that (i) map to the respective methylation site and (ii) have a first score satisfying or not satisfying the threshold value; and (c) assigning the respective methylation site as a quotient of the first number and the second number.

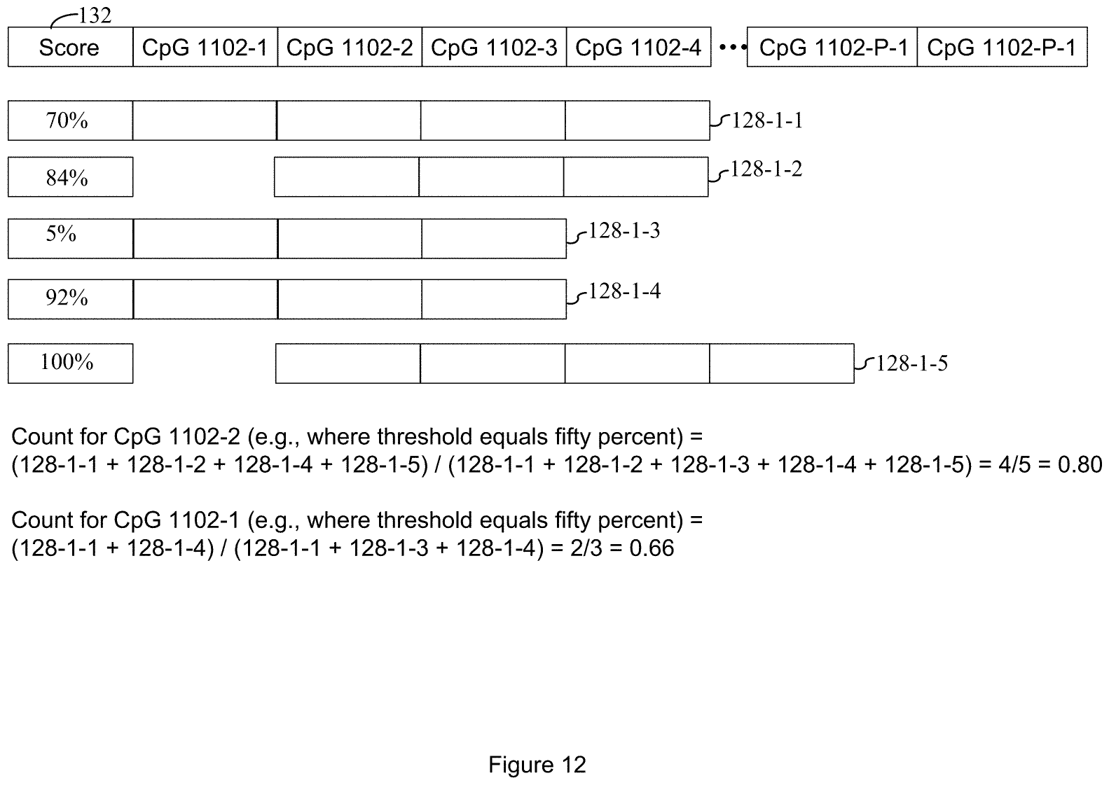

FIG. 12 illustrates. In FIG. 12, one of the methylation sites in the first predetermined set of methylation sites for the first cell source is CpG 1102-2 and there are five nucleic acid fragments that map to this methylation site, 128-1-1, 128-1-2, 128-1-3, 128-1-4, and 128-1-5. In the example the threshold value for the nucleic acid fragment score 132 is fifty percent. Of the five nucleic acid fragments 128 that map to CpG 1102-2, four of the nucleic acid fragments have a nucleic acid fragment score 132 that satisfies the fifty percent threshold. Thus the first number is four. Next a determination is made of a second number of nucleic acid fragments in the plurality of nucleic acid fragments that (i) map to the respective methylation site and (ii) have a first score (nucleic acid fragment score 132) satisfying or not satisfying the threshold value. In this case there are five nucleic acid fragments that map to methylation site CpG 1102-2 and that have a first score (nucleic acid fragment score 132) satisfying or not satisfying the threshold value: 128-1-1, 128-1-2, 128-1-3, 128-1-4, and 128-1-5. Thus, the second number is five. In accordance with the example of FIG. 12, the CpG 1102-2 is assigned a count 134 that is the quotient of the first number and the second number ⅘ or 0.80. This value of 0.80 means that eighty percent of the cell-free nucleic acid fragments in the biological sample that map onto CpG 1102-2 are methylated and twenty percent are not methylated.

In FIG. 12, another of the methylation sites in the first predetermined set of methylation sites for the first cell source is CpG 1102-1 and there are three nucleic acid fragments that map to this methylation site, 128-1-1, 128-1-3, and 128-1-4. In the example, the threshold value for the nucleic acid fragment score 132 remains fifty percent. Of the three nucleic acid fragments 128 that map to CpG 1102-1, two of the nucleic acid fragments have a nucleic acid fragment score 132 that satisfies the fifty percent threshold. Thus the first number is two for CpG 1102-1. Next a determination is made of a second number of nucleic acid fragments in the plurality of nucleic acid fragments that (i) map to the respective methylation site 1102-1 and (ii) have a first score (nucleic acid fragment score 132) satisfying or not satisfying the threshold value. In this case there are three nucleic acid fragments that map to methylation site CpG 1102-1 and that have a first score (nucleic acid fragment score 132) satisfying or not satisfying the threshold value: 128-1-1, 128-1-3, and 128-1-4. Thus, the second number, for CpG 1102-1, is three. In accordance with the example of FIG. 12, the CpG 1102-1 is assigned a count 134 that is the quotient of the first number and the second number, 2/6 or 0.67. This value of 0.67 means that sixty-seven percent of the cell-free nucleic acid fragments in the biological sample that map onto CpG 1102-1 are methylated and the remainder are not methylated.

In some embodiments, as illustrated in FIG. 12, each count in the plurality of counts corresponds to a respective quotient.

In some embodiments, the first score is a likelihood and the threshold value is 0.5 in accordance with the illustration of FIG. 12. In alternative embodiments, the threshold value is at least 0.2, at least 0.3, at least 0.4, at least 0.5, at least 0.6, at least 0.7, at least 0.8, at least 0.9, or at least 0.95.

In some embodiments, the first score (nucleic acid fragment score indicating cell source) specifies other mathematical values. For example, in some embodiments, the first score is a percentage and the threshold value is 50%. In alternative embodiments, the threshold value is at least 20%, at least 30%, at least 40%, at least 50%, at least 60%, at least 70%, at least 80%, at least 90%, or at least 95%.

In some embodiments, the error or uncertainty in the nucleic acid fragment call (e.g., as indicated by the nucleic acid fragment score 132) is propagated into the counts by down-weighting the counts by the uncertainty (e.g., in some embodiments, the count for each nucleic acid fragment is multiplied by the score value). See, for example, Bevington and Robinson, “Data Reduction and Error Analysis for the Physical Sciences,” Second Edition, 1992, The McGraw-Hill Companies, Boston, Mass., pp. 41-50, which is hereby incorporated by reference, for disclosure on exemplary methods for determining the error in a dependent variable (e.g., methylation site count 134) that is a function of one or more measured variables (e.g., the nucleic acid fragments score 132 for those nucleic acid fragments that contribute to a particular methylation site count.

Block 226. The method continues by estimating a first instance of the first cell source fraction in the first biological sample using the first plurality of counts 134 by comparing the respective count 134 of each respective methylation site 144 in the first predetermined set of methylation sites represented by the first plurality of counts to a corresponding reference score for the respective methylation site in a first reference set. Each corresponding reference score in the first reference set is obtained by determining a frequency of occurrence of methylation status at the corresponding methylation site that is in line with the methylation status called for by the first cell source at the corresponding methylation site in nucleic acid fragments obtained from the tissue samples or cell-free nucleic acid samples of corresponding reference subjects in the first plurality of reference subjects (associated with the first cell source).

In some embodiments, a single estimated first cell source fraction in the biological sample of the test subject is determined from the respective count 134 of the respective methylation site of each methylation site in the first predetermined set of methylation sites in the biological sample of the test subject determined as described above. For example, consider the case of a single methylation site. Thus, the support for this methylation site in the biological sample (e.g., blood) from the test subject, in the form of the methylation count 134 for this methylation site, is compared to the reference frequency of the same methylation site across the first plurality of reference subjects. The assumption is made that the sole source of methylation at this single methylation site arises from the first cell source. Thus, with this assumption, the single estimated first cell source fraction is computed as the ratio of the support 146 for methylation at the single methylation site in the test subject (the count 134 for this methylation site) to the reference frequency of methylation for the same methylation site in the reference set. For instance, if the count 134 for the methylation site in the biological sample of the test subject is 0.03 and the reference frequency (of methylation) of the same methylation site is 0.10 in the first plurality of reference subjects, the single estimated first cell source fraction is (0.03)/(0.10) or 0.3. In many instances, even the reference subjects do not observe a frequency of aberrant methylation at the respective methylation sites in the first predetermined set of methylation sites because some tumor tissues are not homogenous.

Further consider the case in which the first predetermined set of methylation sites consists of two methylation sites. That is, the case where the first predetermined set of methylation sites consists of a first methylation site and a second methylation site. The count 134 for the first methylation site from the biological sample (e.g., blood) of the test subject is compared to the reference frequency of methylation of the same methylation site in the first plurality of reference subjects for the first cell source. Likewise, the count 134 for the second methylation site in the first predetermined set of methylation sites from the biological sample of the test subject is compared to the reference frequency of the same methylation site in nucleic acid fragments obtained from the first plurality of reference subjects. The assumption is made that the sole source of aberrant methylation occurring at the first and second methylation sites in the cell-free nucleic acid of the test subject arises from the first cell source. Thus, with this assumption, a ratio for the first methylation site is calculated as the count 134 for the first methylation site, computed as disclosed above, to the reference frequency for the methylation site across the plurality of reference subjects. For instance, if the count 134 for the first methylation site is 0.03 in the biological sample of the test subject and the reference frequency of the first methylation site is 0.10 in the first plurality of reference subjects, the ratio for the first methylation site is (0.03)/(0.10) or 0.3. Further, a ratio for the second methylation site is calculated as the count 134 for the second methylation site in the nucleic acid fragments of the biological sample of the test subject, which is computed as described above, to the reference frequency for the second methylation site in the nucleic acid fragments from the first plurality of reference subjects. For instance, if the count 134 for the second methylation site is 5/85 (meaning that out of 85 nucleic acid fragments in the test subject that encompass the loci of the second methylation site, five include the aberrant methylation state associated with the first cell source) in the test subject and the reference frequency of methylation of the second methylation site is 0.12 across the nucleic acid fragments obtained from the first plurality of reference subjects, the ratio for the second methylation site is ( 5/85)/(0.12) or 0.49.

In some embodiments, more than one methylation site is evaluated in this manner and a ratio between the observed count 134 for each methylation site in the biological sample from the test subject and the frequency of the same methylation site across the nucleic acid fragments obtained from the first plurality of reference subjects is computed for each such methylation site. For example, in some embodiments, more than two methylation sites are evaluated in this way. In such embodiments, the examples above are extended in the sense that a ratio between the observed count 134 for each methylation site in the biological sample from the test subject and the frequency (of aberrant methylation indicative of the first cell source) of the same methylation site across the nucleic acid fragments of first plurality of reference subjects is computed for each such methylation site. Indeed, in some embodiments, between two and 200 methylation sites are compared in this way. In other words, in some embodiments, the first predetermined set of methylation sites consists of between two and 200 methylation sites in such embodiments. In some embodiments, the first predetermined set of methylation sites consists of more than 25, 50, 100, 200, 300, 400, 500, 1000, 2000, or 5000 methylation sites, each of which are compared as described above.

In this way, a number of methylation sites k (the first predetermined set of methylation sites) are evaluated using the first plurality of reference subjects, where k is a positive integer (e.g., 2, 3, more than 20, more than 100, more than 200, etc.). This can be expressed as a k-length vector f₁=(f₁₁, f₁₂, . . . , f₁ₖ) of variant frequencies (number of nucleic acid fragments that support aberrant methylation (indicative of the first cell source) at methylation site an over the total number nucleic acid fragments d₁ᵢ mapping to the genomic location corresponding to methylation site a₁ᵢ) for each methylation site in the first predetermined set of methylation sites, where each component f₁ᵢ of f₁ takes a value between zero to one, across the nucleic acid fragments of the first plurality of reference subjects. Thus f₁=(f₁₁, f₁₂, . . . , f₁ₖ) forms a reference set.

Further, the counts 134 for each methylation site in the biological sample from the test subject nucleic acid fragments overlapping the k nucleic acid fragments represented by the vector f₁ are scanned from the biological sample comprising cell-free nucleic acid molecules from the test subject in the manner disclosed above. For each respective methylation location i in the k methylation locations, the total number of nucleic acid fragments (d₂ᵢ) mapping to the genomic location corresponding to the methylation site i (e.g., covering methylation site i) and the number of these nucleic acid fragments 140 matching the variant methylation pattern (a₂ᵢ) for this site i is determined. The measurements d₂ᵢ and a₂ᵢ are non-negative integer values, from which a quotient f₂ᵢ is taken of a₂ᵢ by d₂ᵢ in the form of count 134, in the manner described above in conjunction with block 208 of FIG. 2A. The respective counts 134 for the methylation sites across the first predetermined set of methylation sites from the test subject can be expressed as the k-length vector f₂=(f₂₁, f₂₂, . . . , f₂ₖ) of respective counts 134 for each methylation site in the first predetermined set of methylation sites.

The objective is to determine a single estimated first cell source fraction of the subject from the observed frequency (support 146) of each methylation site in the first predetermined set of methylation sites. In other words, the goal is to determine the single estimated first cell source fraction, using the fraction of mutant methylation states contributed from the first cell source (e.g., tumor) to the biological sample of the test subject. The vector f₁ summarizes the measured aberrant methylation nucleic acid fragment counts across the first predetermined set of methylation sites from the first cell source across the first plurality of reference subjects. The vector and f₂ summarizes the counts 134 for the first predetermined set of methylation sites in the biological sample from the test subject, from which the underlying first cell source fraction is to be inferred. In some embodiments, methylation sites whose methylation state does not clearly associate with the first cell source are excluded from the analysis. In other words, they are excluded from the k methylation sites considered.

In some embodiments, it is assumed that the nucleic acid fragments 126 from the first cell source are generated according to a Poisson Process. For each methylation site i in k, there is observed a₂ᵢ supporting nucleic acid fragment counts (nucleic acid fragments that have the aberrant methylation at methylation site i that is indicative of the first cell source), and it is expected that f₁₁ times d₂₁ supporting nucleic acid fragment counts. For example, for methylation site 1, consider the case where a₂₁ is 100 and d₂₁ is 1000 meaning that, of the 1000 nucleic acid fragments 128 measured from the biological sample containing cell-free nucleic acid of the test subject that overlap the genomic location corresponding methylation site 1, 100 of the nucleic acid fragments 128 support the aberrant methylation state for the methylation site. Further suppose that, from the first plurality of reference subjects, it was determined that the frequency of aberrant methylation at this methylation site (f₁₁) is 0.25. It is expected, therefore, that there be f₁₁ (0.25) times d₂₁ (1000) or 250 read counts.

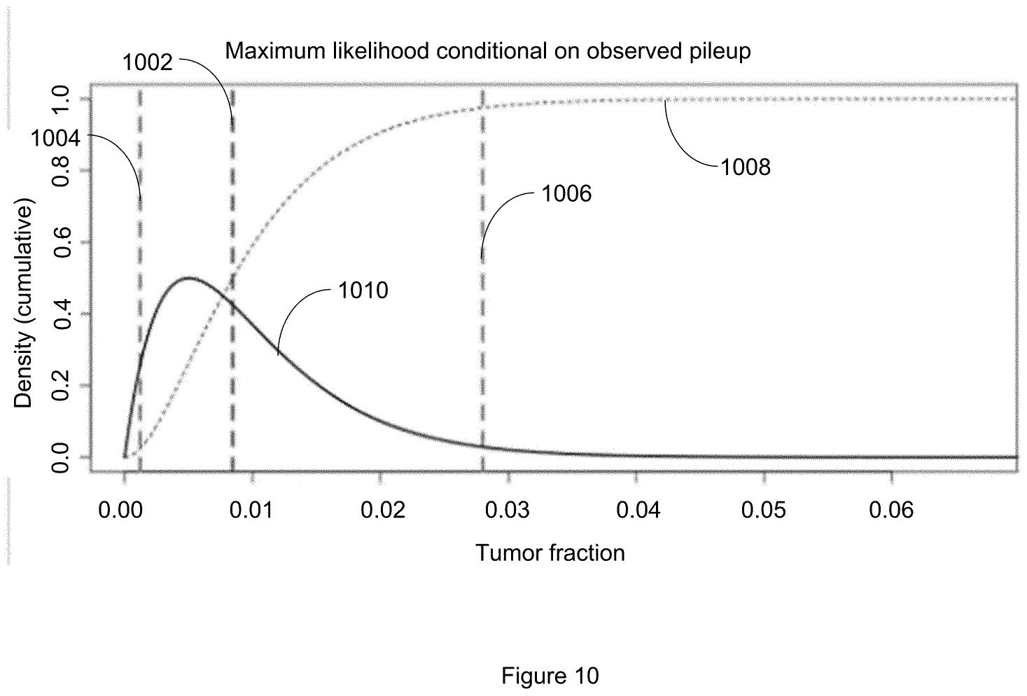

We can thus estimate the cumulative distribution function of the data conditional on t (the rate mutant nucleic acid fragments are contributed from the first cell source to the biological sample containing the cell-free nucleic acid), D(t) to estimate single estimated first cell source fractions corresponding to the 5^{th}, 50^{th} (median), and 95^{th} percentiles using the Poisson model. What is observed in the cell-free DNA biological sample of the test subject is a₂ᵢ supporting nucleic acid fragments for a respective methylation site i in the k methylation sites considered. Further, a calculation of how many sequence nucleic acid fragments supporting the respective methylation site i in the k methylation sites would be expected from the first cell source can be calculated as the variant frequency of the first cell source f₁ᵢ for the respective methylation site i in the first cell source (across the first plurality of reference subjects) multiplied by d₁ᵢ, (the number of sequence nucleic acid fragments mapping to the genomic position covering methylation site i observed in the first cell source) assuming a 100 percent shed rate (meaning that the only source of contribution to the biological sample containing cell-free nucleic acid (e.g., blood sample) is from the first cell source. So, from this t, which can be considered the fraction that converts (i) the expected number of nucleic acid fragments supporting an aberrant methylation state at methylation site i (based on the analysis of the first cell source fraction f₁ᵢ) to (ii) the actual observed number of nucleic acid fragments supporting the aberrant methylation state at methylation site i in the biological sample from the test subject (a₂ᵢ), can be calculated and introduced into a Poisson model and this can be used to estimate a cumulative density function (a probability distribution) that provides an estimate for each trial value oft (where t is sampled from anywhere between zero percent and 110 percent in some embodiments). For instance, if the observed value a₂ᵢ is equal to the expected value, then t would be 100 percent. As another example, if the observed value a₂ᵢ is equal to 110 percent of the expected value, then t would be 110 percent. As still another example, if the observed value a₂ᵢ is equal to 50 percent of the expected value, then t would be 50 percent. Thus, referring to FIG. 10, for each respective trial value oft, all the way from zero to 110 percent, the likelihood of the respective trial value of t is calculated using the cumulative density function (1008). From this, and referring to FIG. 10, the median value for t (the most likely value for t) based on the distribution of likelihoods for t across the range of values of 0 to 110 percent for t (1002), the 5th percentile value for t (lowest value for t, lower bound for t) based of the distribution of likelihoods for t across the range of values of 0 to 110 percent for t (1004), and the 95th percentile (highest value for t, upper bound for t) value for t base on the distribution of likelihoods for t across the range of values of 0 to 110 percent fort (1006), can be calculated. In FIG. 10, the solid line 1010 represents the density function whereas the line 1008 represents the cumulative distribution function. The cumulative distribution function is used to compute the percentile values for t in some embodiments. The 95th percentile value means that an observed fraction of sequence nucleic acid fragments supporting over the total number of sequence nucleic acid fragments overlapping the allele position of a k exceeding the 95^{th} percentile value for t is extremely rate and 95 percent of the time a value for t less than the 95^{th} percentile value for t (about 28 percent in FIG. 10) is expected.

Other bounds, such as the 2^{nd} percentile and 98^{th} percentile, can be used.

The above discussion relates to how t is calculated from the methylation state of a single methylation site. However, as discussed herein, in more common embodiments, multiple methylation sites are sampled, and thus each methylation sites produces an independent likelihood (probability for t) across the range of values (e.g., 0 to 100 percent) considered for t. Thus, the cumulative density function provides a first probability for t at a given trial value oft based on the observed and expected values for variant 1, a second probability for t at the given trial value of t based on the observed and expected values for variant 2, and so forth. To arrive at the cumulative likelihood for t at the given trial value of t, each of the component probabilities (the first probability for t at the given trial value of t based on the observed and expected aberrant methylation state values for methylation site 1, the second probability for t at the given trial value oft based on the observed and expected aberrant methylation state values for methylation state 2, and so forth) are combined and used to compute the cumulative distribution function. In other words, the cumulative distribution function 1008 of FIG. 10 can be drawn using the data from any number of methylation sites based on the assumption that they are independent observations of the same underlying single estimated first cell source fraction. In some embodiments, the probabilities provided by each respective methylation site in the set of k methylation sites for a given trial value oft are combined by adding them together when the probabilities are expressed in logarithmic space to arrive at the computed probability of the trial value for t (the estimated the cell source fraction). In some embodiments, the probabilities provided by each respective methylation site in the set of k methylation sites for a given trial value oft are combined by multiplying them together when the probabilities are expressed in natural scale to arrive at the computed probability of the trial value for t.

In some embodiments, the Poisson model of the likelihood oft across the trial range oft is computed individually for each methylation site k thereby computing a plurality of Poisson models, one for each methylation site. Then the plurality of Poisson models is combined (e.g., summed on log space or multiplied if on the natural scale) for each trial value oft sampled, in order to obtain the likelihood of a trial value oft for each trial value of t sampled. As such, each point in line 1008 is aggregated across the k methylation sites, where k is a positive integer (e.g., 2 or more, 20 or more, 1000 or more). In this way, the most parsimonious explanation of tumor fraction is estimating first cell source fraction as provided.

In some embodiments, the estimated first cell source fraction is taken as the median value for t taken from the distribution of likelihoods for t across the range of values of t sampled using the cumulative density function.

Importantly, this framework enables confidence intervals to be estimated on estimated first cell source fraction in instances in which zero supporting nucleic acid fragments are observed in the test biological sample over the k methylation sites.

As such, the first cell source fraction is estimated conditional on the read information for the set of methylation sites between the (i) biological sample containing the cell-free nucleic acid from the test subject and (ii) the nucleic acid fragments obtained from the respective first tissue sample or the respective first cell-free nucleic acid sample of each corresponding reference subject in the first plurality of reference subjects, where the respective first tissue sample or the respective first cell-free nucleic acid sample corresponds to the first cell source. In this embodiment, therefore, only those methylation sites that are represented in both the test subject and the first plurality of reference subjects are used to compute the single estimated first cell source fraction. In some embodiments the first cell source is a tumor and the estimated first cell source fraction is thus an estimates circulating tumor DNA (ctDNA) fraction.

In alternative embodiments, a negative binomial distribution assumption is assumed rather than a Poisson distribution in order to compute the cumulative distribution function 1008 of FIG. 10.

In some embodiments, the single expected first cell source fraction in the biological sample of the test subject is between 0.5×10⁻⁴ and 1.5×10⁻⁴, and the first cell source is a melanoma. In some embodiments, the single expected first cell source fraction in the biological sample of the test subject is between 0.5×10⁻³ and 1×10⁻², and the first cell source is a renal cancer, uterine cancer, thyroid cancer, prostate cancer, breast cancer, bladder cancer, gastric cancer, cervical cancer or a combination thereof. In some embodiments, the single expected first cell source fraction in the biological sample of the test subject is between 1×10⁻² and 0.8, and the first cell source fraction is lung cancer, esophageal cancer, a head/neck cancer, colorectal cancer, anorectal cancer, ovarian cancer, a hepatobiliary cancer, a pancreatic cancer, or a lymphoma. More discussion on the use of a negative binomial distribution assumptions and Poisson distributions in order to compute the cumulative distribution function is disclosed in International Patent Application No. PCT/US2019/027756, entitled “Systems and Methods for Determining Tumor Fraction in Cell-Free Nucleic Acid,” filed Apr. 16, 2019, which is hereby incorporated by reference.

In some embodiments, a single Poisson model or negative binomial distribution assumption is constructed based on all of the methylation sites in the first reference set (e.g., based on the observed frequency of the methylation statuses for all the methylation sites combined).

In some embodiments, each count of each respective methylation site in the first predetermined set of methylation sites is an observed frequency of methylation of the corresponding methylation site across the first plurality of nucleic acid fragments. In some such embodiments, the estimating further comprises constructing a Poisson model or a negative binomial distribution assumption using the count of each respective methylation site and the corresponding reference frequency of each respective methylation site in the first reference set.

In some embodiments, the Poisson model or the negative binomial distribution assumption is used to form a cumulative density function across a range of calculated first cell source fractions. In some embodiments, the method proceeds by deeming the first cell source fraction to be a mean of the cumulative density function across the range of calculated first cell source fractions.

In some embodiments, a respective Poisson model or negative binomial distribution assumption is constructed for each of the methylation sites in the first reference set.

In some embodiments, each count of each respective methylation site in the first predetermined set of methylation sites is an observed frequency of methylation of the corresponding methylation site across the first plurality of nucleic acid fragments. In such embodiments, the estimating further comprises constructing a respective Poisson model or a respective negative binomial distribution assumption using the count for each respective methylation site and the corresponding reference frequency of the methylation site in the first reference set, thereby constructing a plurality of Poisson models or a plurality of negative binomial distribution assumptions. In some such embodiments, each respective Poisson model or each respective negative binomial distribution assumption is used to form a corresponding cumulative density function across a range of calculated first cell source fractions.

In some embodiments, the method proceeds by deeming the first cell source fraction to be a combination of the mean of the cumulative density function across the range of calculated first cell source fractions combined across the plurality of Poisson models or the plurality of negative binomial distribution assumptions. In some embodiments, the range of calculated first cell source fractions is between zero and 110 percent. In some embodiments, the calculated cell source fraction is at least 0.5 percent, at least 1 percent, at least 2 percent, at least 3 percent, at least 5 percent, at least 7 percent, at least 10 percent, at least 12 percent, at least 15 percent, at least 20 percent, at least 30 percent, at least 40 percent, at least 50 percent, at least 60 percent, at least 70 percent, at least 80 percent, at least 90 percent, at least 100 percent or at least 110 percent.

In some embodiments, the estimated first cell source fraction is used as a basis or a partial basis for determining a stage of a cancer corresponding to the first cell source in the test subject. In some embodiments, the first cell source fraction is used as a basis or a partial basis for determining a treatment option for treating a disease (e.g., a cancer) associated with the first cell source in the test subject. In some embodiments, the first cell source fraction is used as a basis for treatment monitoring.

In some embodiments, given the first cell source fraction, it is possible to determine that certain treatment options are not being effective or will not be effective. For example, checkpoint immunotherapy will not be effective if cytotoxic T-cells are dysfunctional and undergo apoptosis. Such a situation is indicated, for example, when a plurality of nucleic acid fragments is determined to originate from cytotoxic T-cells in the blood. In some embodiments, the estimated first cell source fraction aids in monitoring minimum residual disease amount.

Other ways of estimating first cell source fraction.

In some embodiments, a subject is classified by deeming the subject to have a first condition associated with a first cell source when the observed frequency (support) of aberrant methylation state of each methylation site in a first predetermined set of methylation sites in the genome of a reference sequence of the species satisfies a first threshold. In some embodiments, the first threshold is determined based on a quantification of the reference frequency for aberrant methylation state in methylation sites in the first predetermined set of methylation sites in the genome of a reference sequence of the species. In some embodiments, for instance, the observed frequency of each methylation site in the first predetermined set of methylation sites in the genome of a reference sequence of the species is normalized by the reference frequency (of aberrant methylation) for the corresponding methylation sites in the first predetermined set of methylation sites in the genome of a reference sequence of the species in order to realize an estimated first cell source fraction for the test subject. For instance, in some embodiments, the observed frequency of each methylation site in the first predetermined set of methylation sites in the genome of a reference sequence of the species is divided by the reference frequency (of aberrant methylation state) for the corresponding methylation sites across the first plurality of reference subjects in order to realize the first cell source fraction for the test subject. In this way, the first threshold is determined by a frequency of aberrant methylation state of each methylation site in a first predetermined set of methylation sites in the genome of a reference sequence of the species across the first plurality of reference subjects.

Evaluating first cell source fraction over a time course.

In some embodiments the method further comprises using the estimating of the first cell source fraction at each time point in a plurality of time points (e.g., an epoch) to determine the state or progression (e.g., aggressiveness) of the first cell source in the subject.

In some such embodiments, the method includes obtaining a methylation state of each nucleic acid fragment in a second plurality of nucleic acid fragments in electronic form from a second plurality of cell-free nucleic acid molecules in a second biological sample of the test subject at a second time period.

In some embodiments, the second time period, relative to the first time period, is calibrated for an ability to measure changes in cell-free nucleic acid on the order of hours (e.g., to measure surgery success in removing aberrant tissue from a subject), weeks/months (e.g., to monitor success of therapy for a subject), or years (e.g., to monitor for disease remission in a subject). Thus, in some embodiments, the second time period, relative to the first time period, is a period of months and each time point in the plurality of time points is a different time point in the period of months. In some such embodiments, the period of months is less than four months. In some embodiments, the second time period, relative to the first time period, is a period of years and each time point in the plurality of time points is a different time point in the period of years. In some such embodiments, the period of years is between two and ten years. In some embodiments, the second time period, relative to the first time period, is a period of hours and each time point in the plurality of time points is a different time point in the period of hours. In some such embodiments, the period of hours is between one hour and six hours.

In some embodiments, the second time period is between a month and a year after the first time period. In some embodiments, the second time period is between a day and a week after the first time period. In some embodiments, the second time period is between an hour and a day after the first time period. In some embodiments, the second time period is between one year and five years after the first time period.

The method continues by individually assigning a second score to each respective nucleic acid fragment in the second plurality of nucleic acid fragments, thereby obtaining a plurality of second scores. In some embodiments, each respective second score represents a likelihood that the nucleic acid fragment was obtained from a cell-free nucleic acid molecule that originated from the first cell source.

In some embodiments, the individually assigning comprises: i) comparing the methylation state of the respective nucleic acid fragment against the first canonical set of methylation state vectors and against the second canonical set of methylation state vectors, or ii) presenting the methylation state of the respective nucleic acid fragment to the first classifier.

In some embodiments, the method continues by transforming the plurality of second scores into a second plurality of counts. In some embodiments, each count in the second plurality of counts is for a methylation site in the first predetermined set of methylation sites in the genome of the reference sequence of the species.

In some embodiments, the method continues by estimating a second instance of the first cell source fraction in the second biological sample using the second plurality of counts by comparing the respective count of each respective methylation site in the first predetermined set of methylation sites represented by the second plurality of counts to a corresponding reference score for the respective methylation site in the first reference set.

In some embodiments, the method further comprising using a difference between the first and second instance of the first cell source fraction as a basis or a partial basis for determining an aggressiveness of the first cell source in the test subject. In some embodiments, the method further comprises using methylation features, single nucleotide variants, somatic copy-number alterations, translocations, or other genomic features combined with the difference between the first and second instance of the first cell source fraction as a basis or a partial basis for determining an aggressiveness of the first cell source (e.g., a stage of cancer, an acceleration in metastasis of the cancerous cells).

In some embodiments, the method further comprising using a difference between the first and second instance of the first cell source fraction as a basis or a partial basis for determining a treatment option for the first cell source in the test subject (e.g., a treatment option focused or primarily focused on the cancer state indicated by the presence of the first cell source). In some embodiments, the method further comprises using methylation features, single nucleotide variants, somatic copy-number alterations, translocations, or other genomic features combined with the difference between the first and second instance of the first cell source fraction as a basis or a partial basis for determining a treatment option for the test subject.

In some such embodiments, the method further comprises changing a diagnosis of the subject when the respective instance of the first cell source fraction of the subject is observed to change by a threshold amount over time. For instance, in some embodiments the first cell source fraction at each time point in an epoch is a number between 0 and 1 and, when the first cell source fraction changes by a predetermined amount during the epoch, the diagnosis of the subject is changed. In one example, when the first cell source fraction increases more than two percent, more than three percent, more than four percent, more than five percent, more than ten percent or more than twenty percent during the epoch (e.g., the time period between the first time point at which the first instance of the first cell source fraction was calculated and the second time point at which the second instance of the first cell source fraction was calculated), the diagnosis of the subject is downgraded, indicating that the subject has a more aggressive form of the disease condition and/or a later stage of the disease condition (associated with the first cell source) than initially diagnosed. In another example, when the first cell source fraction decreases more than two percent, more than three percent, more than four percent, more than five percent, more than ten percent or more than twenty percent during the epoch, the diagnosis of the subject is upgraded, indicating that the subject has a less aggressive form of the disease condition and/or an earlier stage of the disease condition associated with the first cell source than initially diagnosed.

In some embodiments, the method further comprises changing a prognosis of the subject when the respective first cell source fraction is observed to change by a threshold amount across an epoch. For instance, in some embodiments the first cell source fraction at each time point in an epoch is a number between 0 and 1 and, when the first cell source fraction changes by a predetermined amount during the epoch the prognosis of the subject is changed. In one example, when the first cell source fraction increases more than two percent, more than three percent, more than four percent, more than five percent, more than ten percent or more than twenty percent during the epoch, the prognosis of the subject is downgraded, indicating that the likelihood of recovery of the subject from the disease condition associated with the first cell source decreases. In another example, when the first cell source fraction decreases more than two percent, more than three percent, more than four percent, more than five percent, more than ten percent or more than twenty percent during the epoch, the prognosis of the subject is upgraded, indicating that the likelihood of recovery of the subject from the disease condition associated with the first cell source improves.

In some embodiments, the method further comprises changing a treatment of the subject when the respective the first cell source fraction is observed to change by a threshold amount across the epoch. For instance, in one example, when the first cell source fraction increases more than two percent, more than three percent, more than four percent, more than five percent, more than ten percent or more than twenty percent during the epoch, the treatment regimen of the subject is changed to a more aggressive treatment. In another example, when the first cell source fraction decreases more than two percent, more than three percent, more than four percent, more than five percent, more than ten percent or more than twenty percent during the epoch, the treatment regimen of the subject is changed to a less aggressive treatment.

In some embodiments, the second biological sample comprises blood, whole blood, plasma, serum, urine, cerebrospinal fluid, fecal, saliva, sweat, tears, pleural fluid, pericardial fluid, and/or peritoneal fluid of the subject. That is, the second biological sample is a mixture of blood, whole blood, plasma, serum, urine, cerebrospinal fluid, fecal, saliva, sweat, tears, pleural fluid, pericardial fluid, and/or peritoneal fluid of the subject and one or more other components of the subject.

In some embodiments, the second biological sample consists of blood, whole blood, plasma, serum, urine, cerebrospinal fluid, fecal, saliva, sweat, tears, pleural fluid, pericardial fluid, and/or peritoneal fluid of the subject. That is, the second biological sample is blood, whole blood, plasma, serum, urine, cerebrospinal fluid, fecal, saliva, sweat, tears, pleural fluid, pericardial fluid, and/or peritoneal fluid of the subject and no other components of the subject.

Defining a classifier. Another aspect of the present disclosure provides a classification method that is performed at a computer system having one or more processors, and memory storing one or more programs for execution by the one or more processors. The method proceeds by obtaining information for each respective reference subject in a first plurality of reference subjects. Each reference subject in the first plurality of reference subjects has a first cell source.

The method proceeds by obtaining a methylation state of each nucleic acid fragment in a first plurality of nucleic acid fragments in electronic form, and using the methylation state of each nucleic acid fragment in the first plurality of nucleic acid fragments to generate a first methylation state vector, thereby obtaining a first canonical set of methylation state vectors.

The method continues by obtaining information for each respective reference subject in a second plurality of reference subjects, wherein each reference subject in the second plurality of reference subjects has a second cell source.

The method proceeds by obtaining a methylation state of each nucleic acid fragment in a second plurality of nucleic acid fragments in electronic form, and using the methylation state of each nucleic acid fragment in the second plurality of nucleic acid fragments to generate a second methylation state vector, thereby obtaining a second canonical set of methylation state vectors.

The method continues by applying the first and second canonical sets of methylation vectors collectively to an untrained or partially trained classifier, in conjunction with cell source of each respective reference subject, thereby obtaining a trained classifier.

In some embodiments, the first cell source is a cell from a cancer and the cancer is one of the set of breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof.

In some embodiments, the classifier determines whether a test subject has a first cell source or is healthy. In some embodiments, the second cell source is from one or more cells in a healthy cancer-free state. In some embodiments, the classifier determines whether a test subject has a first cell source or a second cell source.

In some embodiments, the estimated cell source (e.g., tumor) fraction of the test subject is used as an additional feature of classification.

In some embodiments, the second cell source is distinct from the first cell source, and the second cell source is from one or more cells of breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof.

In some embodiments, each first plurality of nucleic acid fragments is derived from a tissue sample or cell-free nucleic acid sample of a corresponding first reference subject. In some embodiments, each second plurality of nucleic acid fragments is derived from a tissue sample or cell-free nucleic acid sample of a corresponding second reference subject.

In some embodiments, the classifier is based on a neural network algorithm, a support vector machine algorithm, a decision tree algorithm, an unsupervised clustering algorithm, a supervised clustering algorithm, or a logistic regression algorithm, a mixture model, or a hidden Markov model. In some embodiments, the trained classifier is a multinomial classifier.

In some embodiments the classifier makes use of the B score classifier described in United States Patent Publication No. 62/642,461, entitled “Method and System for Selecting, Managing, and Analyzing Data of High Dimensionality,” filed 62/642,461, which is hereby incorporated by reference.

In some embodiments, the classifier makes use of the M score classifier described in U.S. Patent Application No. 62/642,480, entitled “Methylation Fragment Anomaly Detection,” filed Mar. 13, 2018, which is hereby incorporated by reference.

In some embodiments, the classifier is a neural network or a convolutional neural network. See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. See also, U.S. Patent Application No. 62/679,746, entitled “Convolutional Neural Network Systems and Methods for Data Classification,” filed Jun. 1, 2018, which is hereby incorporated by reference, for its disclosure of convolutional neural networks that can be used for classifying methylation patterns in accordance with the present disclosure.

In some embodiments, the classifier is a support vector machine (SVM). SVMs are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space corresponds to a non-linear decision boundary in the input space.

In some embodiments, the classifier is a decision tree. Decision trees are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety.

In some embodiments, the classifier is an unsupervised clustering model. In some embodiments, the classifier is a supervised clustering model. Clustering is described at pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (e.g., similarity measure) is used to ensure that the samples in one cluster are more like one another than they are to samples in other clusters. Second, a mechanism for partitioning the data into clusters using the similarity measure is determined. Similarity measures are discussed in Section 6.7 of Duda 1973, where it is stated that one way to begin a clustering investigation is to define a distance function and to compute the matrix of distances between all pairs of samples in the training set. If distance is a good measure of similarity, then the distance between reference entities in the same cluster will be significantly less than the distance between the reference entities in different clusters. However, as stated on page 215 of Duda 1973, clustering does not require the use of a distance metric. For example, a nonmetric similarity function s(x, x′) can be used to compare two vectors x and x′. Conventionally, s(x, x′) is a symmetric function whose value is large when x and x′ are somehow “similar.” An example of a nonmetric similarity function s(x, x′) is provided on page 218 of Duda 1973. Once a method for measuring “similarity” or “dissimilarity” between points in a dataset has been selected, clustering requires a criterion function that measures the clustering quality of any partition of the data. Partitions of the data set that extremize the criterion function are used to cluster the data. See page 217 of Duda 1973. Criterion functions are discussed in Section 6.8 of Duda 1973. More recently, Duda et al., Pattern Classification, 2^{nd} edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J., each of which is hereby incorporated by reference. Particular exemplary clustering techniques that can be used in the present disclosure include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering where no preconceived notion of what clusters should form when the training set is clustered are imposed.

In some embodiments, the classifier is a regression model, such as the multi-category logit models described in Agresti, An Introduction to Categorical Data Analysis, 1996, John Wiley & Sons, Inc., New York, Chapter 8, which is hereby incorporated by reference in its entirety. In some embodiments, the classifier makes use of a regression model disclosed in Hastie et al., 2001, The Elements of Statistical Learning, Springer-Verlag, New York.

In some embodiments, the classifier is a Naive Bayes algorithm, such as the tool developed by Rosen et al. to deal with metagenomic reads (See, Bioinformatics 27(1):127-129, 2011). In some embodiments, the classifier is a nearest neighbor algorithm, such as the non-parametric methods described by Kamvar et al., Front Genetics 6:208 doi: 10.3389/fgene.2015.00208, 2015). In some embodiments, the classifier is a mixture model, such as that described in McLachlan et al., Bioinformatics 18(3):413-422, 2002. In some embodiments, in particular those embodiments including a temporal component, the classifier is a hidden Markov model such as described by Schliep et al., 2003, Bioinformatics 19(1):i255-i263.

### Additional Embodiments

Determining an estimated fraction for a test subject with respect to a third cell source. In some embodiments, the method analyzes the nucleic acid fragments of the test subject in cases where the second cell source is a second cancer type or a second cancer stage.

In some embodiments, the individually assigning further assigns a second score to each respective nucleic acid fragment in the first plurality of nucleic acid fragments, thereby obtaining a plurality of second scores. Each respective second score in the plurality of second scores for a nucleic acid fragment in the first plurality of nucleic acid fragments. Each respective second score represents a likelihood that the nucleic acid fragment was obtained from a cell-free nucleic acid molecule that originated from a circulating tumor nucleic acid associated with a third cell source.

In some embodiments, the individually assigning compares the methylation state of the respective nucleic acid fragment against a third canonical set of methylation state vectors and against the second canonical set of methylation state vectors, or a second classifier trained at least in part on the third canonical set of methylation state vectors and the second canonical set of methylation state vectors. Each canonical methylation state vector in the third canonical set of methylation state vectors is derived from a tissue sample or cell-free nucleic acid sample of a corresponding reference subject in a third plurality of reference subjects corresponding to the third cell source.

In some embodiments, the transforming further comprises transforming the second plurality of scores into a second plurality of counts. Each count in the second plurality of counts is for a methylation site in a second predetermined set of methylation sites in the genome of a reference sequence of the species. The second predetermined set of methylation sites is associated with the third cell source.

In some embodiments, the method further comprises estimating a second cell source or tumor fraction, with respect to the second cell source, in the test subject using the second plurality of counts. The method proceeds by comparing the respective count of each respective methylation site in the second predetermined set of methylation sites represented by the second plurality of counts to a corresponding reference score for the respective methylation site in a second reference set. Each corresponding reference score in the second reference set is obtained by determining a frequency of methylation of the corresponding methylation site in nucleic acid fragments obtained from the tissue sample or cell-free nucleic acids of a corresponding reference subject in the third plurality of reference subjects.

In some embodiments, the individually assigning compares the methylation state of the respective nucleic acid fragment against the second classifier. In some embodiments, the first classifier and the second classifier are the same, and the first classifier is trained at least in part on the first canonical set of methylation state vectors, the second canonical set of methylation state vectors, and the third canonical set of methylation state vectors.

In some embodiments, the first classifier is other than the second classifier and the first classifier is not trained on the third canonical set of methylation state vectors.

Determining estimated cell fractions for a test subject with respect to a plurality of cell sources. Another aspect of the present disclosure provides for a method for estimating cell source (e.g., tumor) fraction with respect to each cell source in a plurality of cell sources in a test subject of a given species. The method comprises obtaining in electronic form a methylation state of each nucleic acid fragment in a first plurality of nucleic acid fragments from a first plurality of cell-free nucleic acid molecules in a first biological sample of the test subject at a first time period.

In some embodiments, the method proceeds by individually assigning a plurality of scores to each respective nucleic acid fragment in the first plurality of nucleic acid fragments, thereby obtaining a first plurality of score sets. In some embodiments, each set includes a plurality of scores each corresponding to a cell source in the plurality of cell sources. In some embodiments, each respective score set in the first plurality of score sets is for a corresponding nucleic acid fragment in the first plurality of nucleic acid fragments. In some embodiments, each respective score in each respective score set, in the plurality of score sets, represents a likelihood that the corresponding nucleic acid fragment was obtained from a cell-free nucleic acid molecule that originated from a circulating tumor nucleic acid associated with a corresponding different cell source in the plurality of cell sources. In some embodiments, the individually assigning compares the methylation state of the respective nucleic acid fragment against a plurality of canonical sets of methylation state vectors, or a classifier trained at least in part on the plurality of canonical sets of methylation state vectors. In some embodiments, each canonical methylation state vector in each canonical set of methylation state vectors in the plurality of canonical sets of methylation state vectors is derived from a tissue sample or cell-free nucleic acid sample of a corresponding reference subject in a plurality of reference subjects. In some embodiments, the plurality of reference subjects includes at least one representative reference subject for each respective cell source in the plurality of cell sources.

In some embodiments, the method continues by transforming the plurality of scores sets into a plurality of count sets, wherein each respective count set in the plurality of count sets represents a different cell source in the plurality of cell sources. In some embodiments, for each respective count set, each count in the respective count set is for a methylation site in a corresponding predetermined set of methylation sites in the genome of a reference sequence of the species that corresponds to the cell source represented by the respective count set.

In some embodiments, the method continues by estimating the plurality of cell source fractions, each respective cell source fraction in the plurality of cell source fractions being with respect to a corresponding cell source in the plurality of cell sources, in the test subject using the plurality of count sets. In some embodiments, the estimating comprises, for each respective count set in the plurality of count sets, comparing the respective count of each respective methylation site in the predetermined set of methylation sites corresponding to the count set to a corresponding reference score for the respective methylation site in a corresponding reference set. In some embodiments, each corresponding reference score in the corresponding reference set is obtained by determining a frequency of methylation of the corresponding methylation site in nucleic acid fragments obtained from a tissue sample or cell-free nucleic acids of a corresponding reference subject in the plurality of reference subjects corresponding to the cell source represented by the count set.

In some embodiments, the first cancer type can be the same as the second cancer type. Alternatively, the first cancer type can be different than the second cancer type. In some embodiments, the first cancer type and the second cancer type are each selected from the group consisting of breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, and gastric cancer.

### EXAMPLE 1—INCREASE IN MEDIAN ctDNA FRACTION BY CANCER BY STAGE

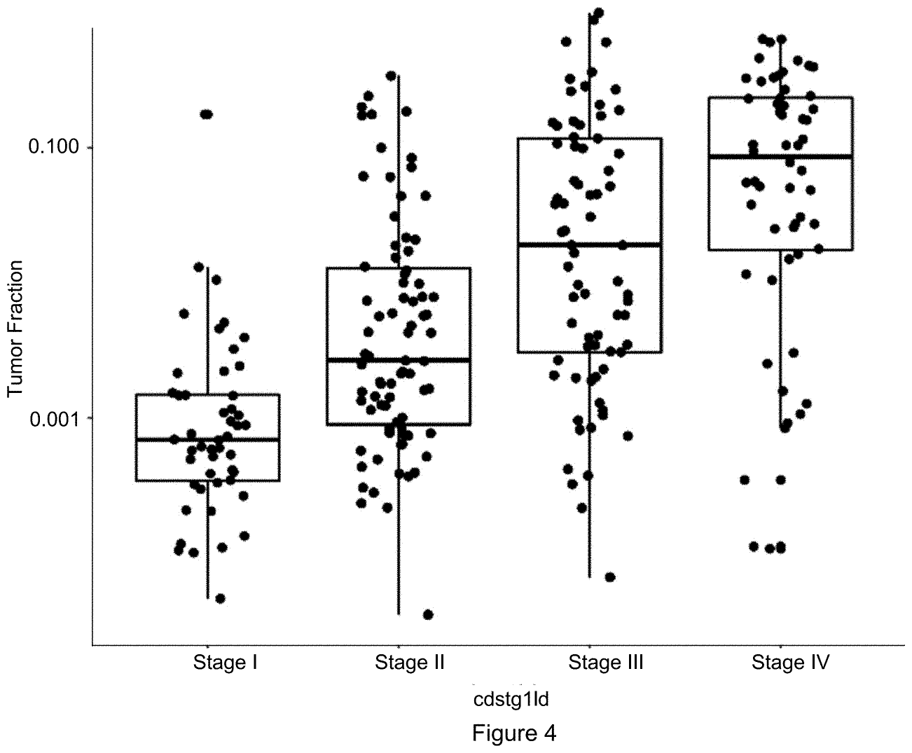

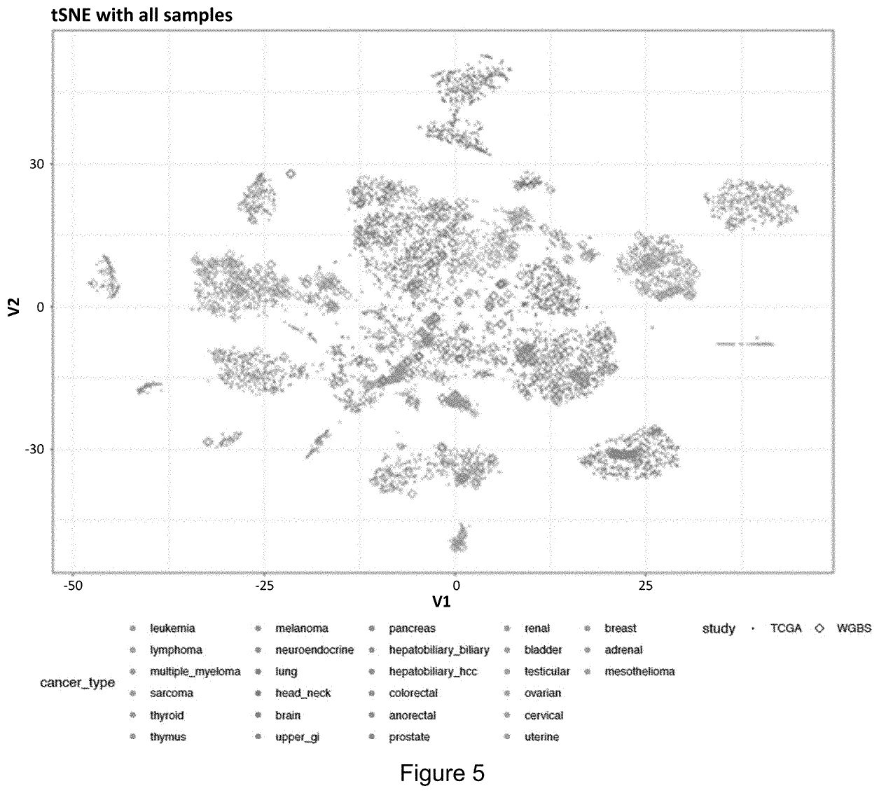

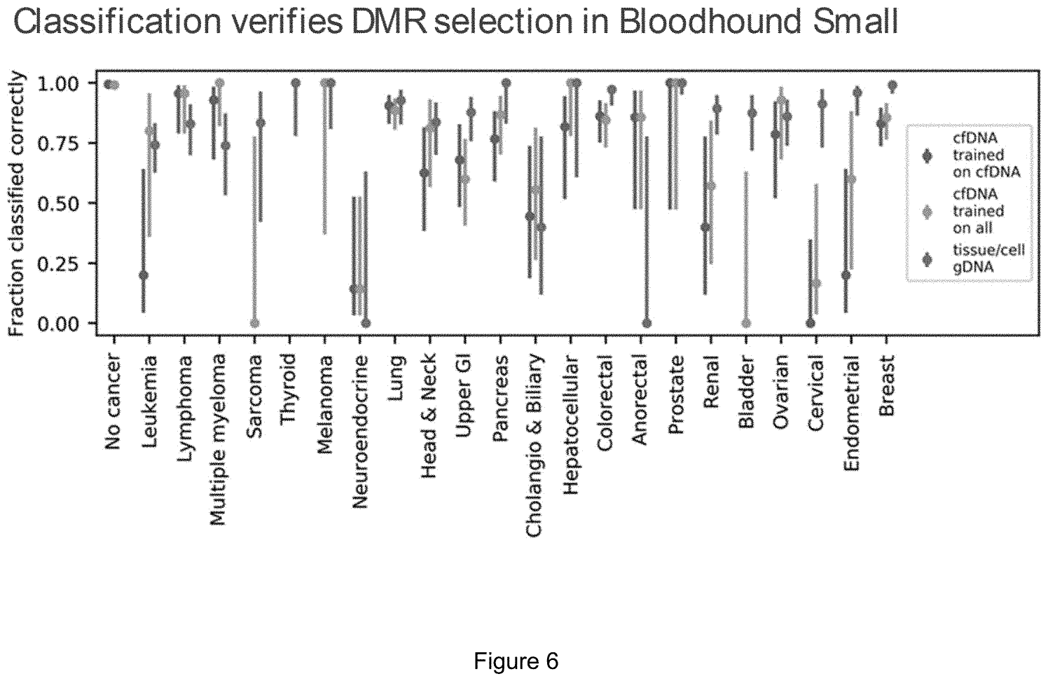

Referring to FIG. 4, subjects are grouped by cancer stages I, II, III, and IV, regardless of the type of cancer that they have. In FIG. 4, the x-axis indicates which cancer stage each subject has and while the y-axis indicates the observed ctDNA fraction for each subject. The method used to compute the cfDNA fraction for each subject comprises obtaining a first plurality of nucleic acid fragments 128 in electronic form from a biological sample of each subject in a cohort, where the biological sample comprises cell-free nucleic acid molecules

FIG. 4 provides an analysis of how ctDNA fraction varies by cancer stage regardless of cancer type, among subjects that have cell-free nucleic acid fragments that indicate their underlying cancer. FIG. 4 thus shows that, as the disease is more severe as determined by clinically staging (stages 1 through 4), more evidence of tumor fraction (larger ctDNA fraction) is found in the cfDNA. While FIG. 4 shows that while this is the general case across the CCGA cohort (see Example 6 for details of the CCGA cohort), there are violations (outliers) to this trend. Such outliers in FIG. 4 are suggestive and best explained by clinical misclassification. FIG. 4 thus shows a fundamental component of the underlying disease, which is general expected tumor fraction rates in the cfDNA. FIG. 4 also shows that stage 4 has some individuals that have very low shedding rates indicating that there are different sub-states within stage 4.

FIG. 4 illustrates that shedding rates (ctDNA fraction) can be used as a basis for establishing meaningful and informative thresholds.

### EXAMPLE 2—OBTAINING A PLURALITY OF NUCLEIC ACID FRAGMENTS

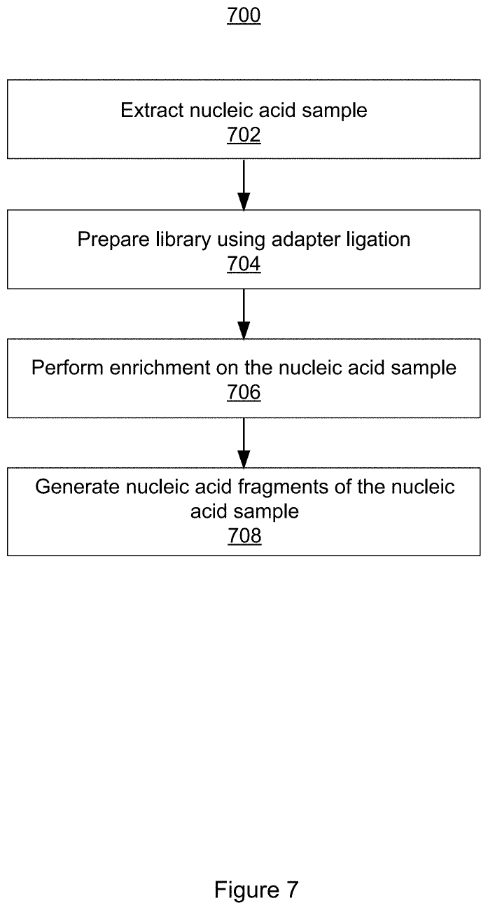

FIG. 7 is a flowchart of method 700 for preparing a nucleic acid sample for sequencing according to one embodiment. The method 700 includes, but is not limited to, the following steps. For example, any step of method 700 may comprise a quantitation sub-step for quality control or other laboratory assay procedures known to one skilled in the art.

In block 702, a nucleic acid sample (DNA or RNA) is extracted from a subject. The sample may be any subset of the human genome, including the whole genome. The sample may be extracted from a subject known to have or suspected of having cancer. The sample may include blood, plasma, serum, urine, fecal, saliva, other types of bodily fluids, or any combination thereof. In some embodiments, methods for drawing a blood sample (e.g., syringe or finger prick) may be less invasive than procedures for obtaining a tissue biopsy, which may require surgery. The extracted sample may comprise cfDNA and/or ctDNA. For healthy individuals, the human body may naturally clear out cfDNA and other cellular debris. If a subject has a cancer or disease, ctDNA in an extracted sample may be present at a detectable level for diagnosis.

In block 704, a sequencing library is prepared. During library preparation, unique molecular identifiers (UMI) are added to the nucleic acid molecules (e.g., DNA molecules) through adapter ligation. The UMIs are short nucleic acid sequences (e.g., 4-10 base pairs) that are added to ends of DNA fragments during adapter ligation. In some embodiments, UMIs are degenerate base pairs that serve as a unique tag that can be used to identify sequence reads originating from a specific DNA fragment. During PCR amplification following adapter ligation, the UMIs are replicated along with the attached DNA fragment. This provides a way to identify sequence reads that came from the same original fragment in downstream analysis.

In block 706, targeted DNA sequences are enriched from the library. During enrichment, hybridization probes (also referred to herein as “probes”) are used to target, and pull down, nucleic acid fragments informative for the presence or absence of cancer (or disease), cancer status, or a cancer classification (e.g., cancer class or tissue of origin). For a given workflow, the probes may be designed to anneal (or hybridize) to a target (complementary) strand of DNA. The target strand may be the “positive” strand (e.g., the strand transcribed into mRNA, and subsequently translated into a protein) or the complementary “negative” strand. The probes may range in length from 10s, 100s, or 1000s of base pairs. In one embodiment, the probes are designed based on a methylation site panel. In one embodiment, the probes are designed based on a panel of targeted genes to analyze particular mutations or target regions of the genome (e.g., of the human or another organism) that are suspected to correspond to certain cancers or other types of diseases. Moreover, the probes may cover overlapping portions of a target region. In block 708, these probes are used to generate sequence reads of the nucleic acid sample.

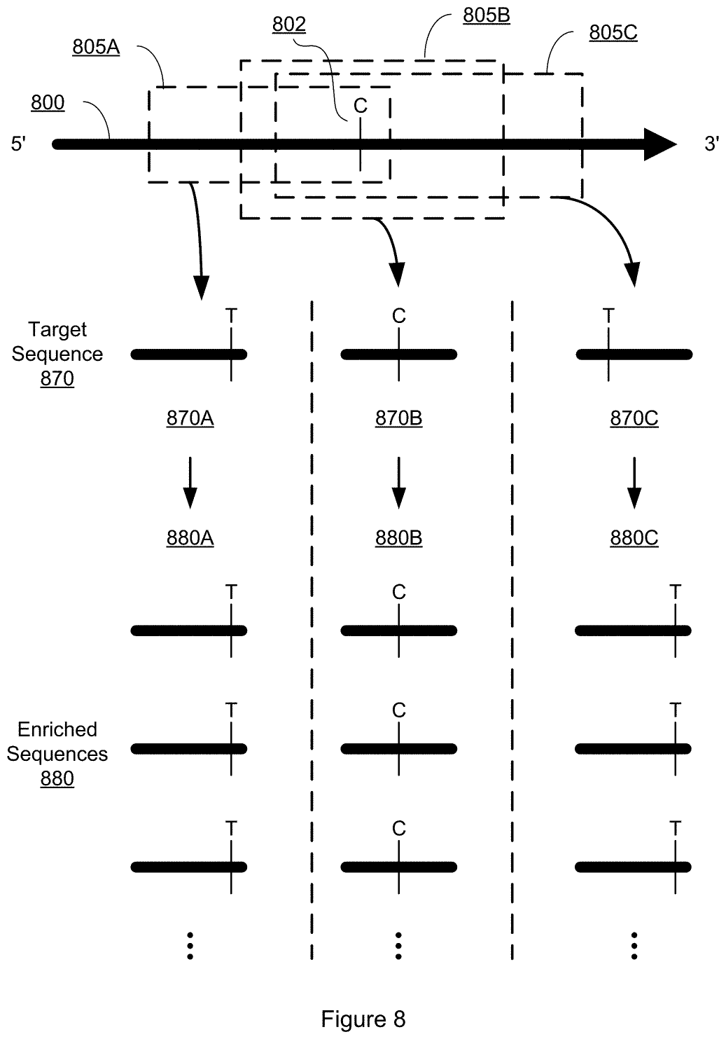

FIG. 8 is a graphical representation of the process for obtaining sequence reads from the nucleic acid sample according to one embodiment. FIG. 8 depicts one example of a nucleic acid segment 800 from the biological sample. Here, the nucleic acid segment 800 can be a single-stranded nucleic acid segment, such as a single stranded. In some embodiments, the nucleic acid segment 800 is a double-stranded cfDNA segment. The illustrated example depicts three regions 805A, 805B, and 805C of the nucleic acid segment that can be targeted by different probes. Specifically, each of the three regions 805A, 805B, and 805C includes an overlapping position on the nucleic acid segment 800. An example overlapping position is depicted in FIG. 8 as the cytosine (“C”) nucleotide base 802. The cytosine nucleotide base 802 is located near a first edge of region 805A, at the center of region 805B, and near a second edge of region 805C.

In some embodiments, one or more (or all) of the probes are designed based on a gene panel or methylation site panel to analyze particular mutations or target regions of the genome (e.g., of the human or another organism) that are suspected to correspond to certain cancers or other types of diseases. By using a targeted gene panel or methylation site panel rather than sequencing all expressed genes of a genome, also known as “whole exome sequencing,” the method 800 may be used to increase sequencing depth of the target regions, where depth refers to the count of the number of times a given target sequence within the sample has been sequenced. Increasing sequencing depth reduces required input amounts of the nucleic acid sample.

Hybridization of the nucleic acid sample 800 using one or more probes results in an understanding of a target sequence 870. As shown in FIG. 8, the target sequence 870 is the nucleotide base sequence of the region 805 that is targeted by a hybridization probe. The target sequence 870 can also be referred to as a hybridized nucleic acid fragment. For example, target sequence 870A corresponds to region 805A targeted by a first hybridization probe, target sequence 870B corresponds to region 805B targeted by a second hybridization probe, and target sequence 870C corresponds to region 805C targeted by a third hybridization probe. Given that the cytosine nucleotide base 802 is located at different locations within each region 805A-C targeted by a hybridization probe, each target sequence 870 includes a nucleotide base that corresponds to the cytosine nucleotide base 802 at a particular location on the target sequence 870.

After a hybridization step, the hybridized nucleic acid fragments are captured and may also be amplified using PCR. For example, the target sequences 870 can be enriched to obtain enriched sequences 880 that can be subsequently sequenced. In some embodiments, each enriched sequence 880 is replicated from a target sequence 870. Enriched sequences 880A and 880C that are amplified from target sequences 870A and 870C, respectively, also include the thymine nucleotide base located near the edge of each sequence read 880A or 880C. As used hereafter, the mutated nucleotide base (e.g., thymine nucleotide base) in the enriched sequence 880 that is mutated in relation to the reference allele (e.g., cytosine nucleotide base 802) is considered as the alternative allele. Additionally, each enriched sequence 880B amplified from target sequence 870B includes the cytosine nucleotide base located near or at the center of each enriched sequence 880B.

In block 708, sequence reads are generated from the enriched DNA sequences, e.g., enriched sequences 880 shown in FIG. 8. Sequencing data may be acquired from the enriched DNA sequences by known means in the art. For example, the method 800 may include next generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators.

In some embodiments, the sequence reads may be aligned to a reference genome using known methods in the art to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene.

In various embodiments, a sequence read is comprised of a read pair denoted as R₁ and R₂. For example, the first read R₁ may be sequenced from a first end of a nucleic acid fragment whereas the second read R₂ may be sequenced from the second end of the nucleic acid fragment. Therefore, nucleotide base pairs of the first read R₁ and second read R₂ may be aligned consistently (e.g., in opposite orientations) with nucleotide bases of the reference genome. Alignment position information derived from the read pair R₁ and R₂ may include a beginning position in the reference genome that corresponds to an end of a first read (e.g., R₁) and an end position in the reference genome that corresponds to an end of a second read (e.g., R₂). In other words, the beginning position and end position in the reference genome represent the likely location within the reference genome to which the nucleic acid fragment corresponds. An output file having SAM (sequence alignment map) format or BAM (binary) format may be generated and output for further analysis such as methylation state determination.

### EXAMPLE 3—ABILITY TO DETECT CANCER AS A FUNCTION OF cfDNA FRACTION

The A score classifier, described herein is a classifier of tumor mutational burden based on targeted sequencing analysis of nonsynonymous mutations. For example, a classification score (e.g., “A score”) can be computed using logistic regression on tumor mutational burden data, where an estimate of tumor mutational burden for each individual is obtained from the targeted cfDNA assay. In some embodiments, a tumor mutational burden can be estimated as the total number of variants per individual that are: called as candidate variants in the cfDNA, passed noise-modeling and joint-calling, and/or found as nonsynonymous in any gene annotation overlapping the variants. The tumor mutational burden numbers of a training set can be fed into a penalized logistic regression classifier to determine cutoffs at which 95% specificity is achieved using cross-validation. Additional details on A score can be found, for example, in R. Chaudhary et al., 2017, “Journal of Clinical Oncology, 35(5), suppl.e14529, pre-print online publication, which is hereby incorporated by reference herein in its entirety.

The B score classifier is described in United States Patent Publication No. 62/642,461, filed 62/642,461, which is hereby incorporated by reference. In accordance with the B score method, a first set of nucleic acid fragments of nucleic acid samples from healthy subjects in a reference group of healthy subjects are analyzed for regions of low variability. Accordingly, each nucleic acid fragment in the first set of nucleic acid fragments of nucleic acid samples from each healthy subject are aligned to a region in the reference genome. From this, a training set of nucleic acid fragments from nucleic acid fragments of nucleic acid samples from subjects in a training group are selected. Each nucleic acid fragment in the training set aligns to a region in the regions of low variability in the reference genome identified from the reference set. The training set includes nucleic acid fragments of nucleic acid samples from healthy subjects as well as nucleic acid fragments of nucleic acid samples from diseased subjects who are known to have the cancer. The nucleic acid samples from the training group are of a type that is the same as or similar to that of the nucleic acid samples from the reference group of healthy subjects. From this it is determined, using quantities derived from nucleic acid fragments of the training set, one or more parameters that reflect differences between nucleic acid fragments of nucleic acid samples from the healthy subjects and nucleic acid fragments of nucleic acid samples from the diseased subjects within the training group. Then, a test set of nucleic acid fragments s associated with nucleic acid samples comprising cfNA fragments from a test subject whose status with respect to the cancer is unknown is received, and the likelihood of the test subject having the cancer is determined based on the one or more parameters.

The M score classifier is described in U.S. Patent Application No. 62/642,480, entitled “Methylation Fragment Anomaly Detection,” filed Mar. 13, 2018, which is hereby incorporated by reference.

### EXAMPLE 4—PRECISION OF A WHOLE-GENOME BISULFITE SEQUENCING MULTI-CLASS CANCER TYPE CLASSIFIER AS A FUNCTION OF cfDNA FRACTION

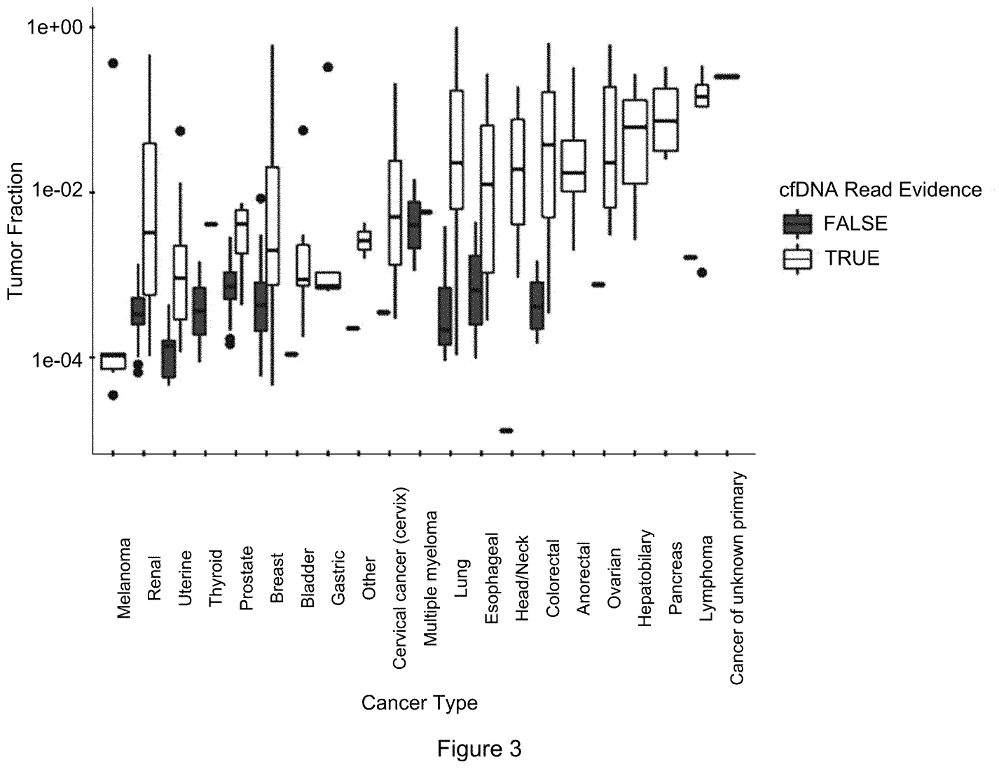

FIG. 8 details the precision of a multi-class classifier for the CCGA cohort of subjects (Example 6 below) that have been sequenced using whole genome bisulfite sequencing (WGBS) spanning the spectrum of different cancers identified in FIG. 3 as a function of ctDNA fraction. For details regarding WGBS, see, for example, Example 7. See also, U.S. Patent Application No. 62/642,480, entitled “Methylation Fragment Anomaly Detection,” filed Mar. 13, 2018, which is hereby incorporated by reference. As illustrated in FIG. 8, the cohort is binned into eight different cfDNA fraction bins and the precision, defined as the ability to place the correct cancer for a given subject into the top two cancer class probabilities, of the WGBS classifier for each such bin, and the number of subjects in the cohort in each such bin is provided. FIG. 8 suggests that a threshold ctDNA fraction level is needed in order to achieve the correct assignment using the WGBS multi-class cancer type classifier.

### EXAMPLE 5—POSITIVE ASSOCIATION OF ctDNA FRACTION WITH TUMOR SIZE

FIG. 10 illustrates the positive association of tumor size with ctDNA fraction, across all stages of cancer using the CCGA cohort described in Example 6. Since tumor size is positively associated with cancer aggressiveness in many instances, Example 5 provides additional support for the use of cfDNA fraction to classify subjects in accordance with the present disclosure, including the methods disclosed in conjunction with FIG. 2, the additional embodiments disclosed below, and the claims of the present disclosure.

### EXAMPLE 6—CELL-FREE GENOME ATLAS STUDY (CCGA) COHORT

Subjects from the CCGA [NCT02889978] were used in the Examples of the present disclosure. CCGA is a prospective, multi-center, observational cfDNA-based early cancer detection study that has enrolled over 15,000 demographically-balanced participants at over 140 sites.

This example looks at one of the sub-studies of CCGA. Blood was collected from subjects with newly diagnosed therapy-naive cancer (C, case) and participants without a diagnosis of cancer (noncancer [NC], control) as defined at enrollment. This preplanned sub-study included 878 cases, 580 controls, and 169 assay controls (n=1627) across twenty tumor types and all clinical stages.

All samples were analyzed by: 1) paired cfDNA and white blood cell (WBC)-targeted sequencing (60,000X, 507 gene panel); a joint caller removed WBC-derived somatic variants and residual technical noise; 2) paired cfDNA and WBC whole-genome sequencing (WGS; 35X); a novel machine learning algorithm generated cancer-related signal scores; joint analysis identified shared events; and 3) cfDNA whole-genome bisulfate sequencing (WGBS; 34X); normalized scores were generated using abnormally methylated fragments. In the targeted assay, non-tumor WBC-matched cfDNA somatic variants (SNVs/indels) accounted for 76% of all variants in NC and 65% in C. Consistent with somatic mosaicism (e.g., clonal hematopoiesis), WBC-matched variants increased with age; several were non-canonical loss-of-function mutations not previously reported. After WBC variant removal, canonical driver somatic variants were highly specific to C (e.g., in EGFR and PIK3CA, 0 NC had variants vs 11 and 30, respectively, of C). Similarly, of 8 NC with somatic copy number alterations (SCNAs) detected with WGS, four were derived from WBCs. WGBS data of the CCGA reveals informative hyper- and hypo-fragment level CpGs (1:2 ratio); a subset of which was used to calculate methylation scores. A consistent “cancer-like” signal was observed in <1% of NC participants across all assays (representing potential undiagnosed cancers). An increasing trend was observed in NC vs stages I-III vs stage IV (nonsyn. SNVs/indels per Mb [Mean±SD] NC: 1.01±0.86, stages I-III: 2.43±3.98; stage IV: 6.45±6.79; WGS score NC: 0.00±0.08, I-III: 0.27±0.98; IV: 1.95±2.33; methylation score NC: 0±0.50; I-III: 1.02±1.77; IV: 3.94±1.70). These data demonstrate the feasibility of achieving >99% specificity for invasive cancer, and support the promise of cfDNA assay for early cancer detection.

### EXAMPLE 7—GENERATION OF METHYLATION STATE VECTOR

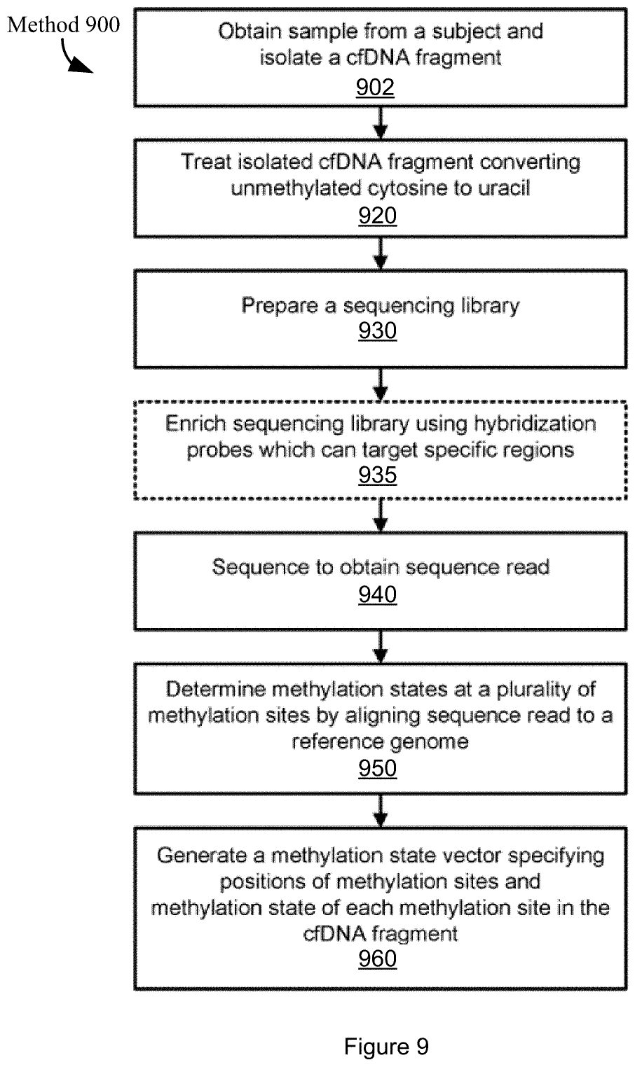

FIG. 9 is a flowchart describing a process 900 of sequencing a fragment of cfDNA to obtain a methylation state vector, according to an embodiment in accordance with the present disclosure.

Referring to step 902, the cfDNA fragments are obtained from the biological sample (e.g., as discussed above in conjunction with FIG. 2). Referring to step 920, the cfDNA fragments are treated to convert unmethylated cytosines to uracils. In one embodiment, the DNA is subjected to a bisulfite treatment that converts the unmethylated cytosines of the fragment of cfDNA to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, Calif.)) is used for the bisulfite conversion in some embodiments. In other embodiments, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, Mass.). In some embodiments, methylated cytosines can be converted to uracils via enzymatic conversion as well.

From the converted cfDNA fragments, a sequencing library is prepared (step 930). In some embodiments, the sequencing library is enriched 935 for cfDNA fragments, or genomic regions, that are informative for cancer status using a plurality of hybridization probes; for example, in a targeted methylation sequencing assay. The hybridization probes are short oligonucleotides capable of hybridizing to particularly specified cfDNA fragments, or targeted regions, and enriching for those fragments or regions for subsequent sequencing and analysis. Hybridization probes may be used to perform a targeted, high-depth analysis of a set of specified CpG sites of interest to the researcher. Once prepared, the sequencing library or a portion thereof can be sequenced to obtain a plurality of nucleic acid fragments. The nucleic acid fragments may be in a computer-readable, digital format for processing and interpretation by computer software.

From the fragments, a location and methylation state for each of CpG site is determined based on alignment of the nucleic acid fragments to a reference genome (950). A methylation state vector for each fragment specifies information such as a location of the fragment in the reference genome (e.g., as specified by the position of the first CpG site in each fragment, or another similar metric), a number of CpG sites in the fragment, and the methylation state of each CpG site in the fragment (960).

### EXAMPLE 8—EXAMPLE CELL SOURCES

In some embodiments, a cell source of any embodiment of the present disclosure is a first cancer of a common primary site of origin. In some embodiments, the first cancer is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof.

In some embodiments, a cell source of any embodiment of the present disclosure is a tumor of a certain cancer type, or a fraction thereof. In some embodiments, the tumor is an adrenocortical carcinoma, a childhood adrenocortical carcinoma, a tumor of an AIDS-related cancer, kaposi sarcoma, a tumor associated with anal cancer, a tumor associated with an appendix cancer, an astrocytoma, a childhood (brain cancer) tumor, an atypical teratoid/rhabdoid tumor, a central nervous system (brain cancer) tumor, a basal cell carcinoma of the skin, a tumor associated with bile duct cancer, a bladder cancer tumor, a childhood bladder cancer tumor, a bone cancer (e.g., ewing sarcoma and osteosarcoma and malignant fibrous histiocytoma) tissue, a brain tumor, breast cancer tissue, childhood breast cancer tissue, a childhood bronchial tumor, burkitt lymphoma tissue, a carcinoid tumor (gastrointestinal), a childhood carcinoid tumor, a carcinoma of unknown primary, a childhood carcinoma of unknown primary, a childhood cardiac (heart) tumor, a central nervous system (e.g., brain cancer such as childhood atypical teratoid/rhabdoid) tumor, a childhood embryonal tumor, a childhood germ cell tumor, cervical cancer tissue, childhood cervical cancer tissue, cholangiocarcinoma tissue, childhood chordoma tissue, a chronic myeloproliferative neoplasm, a colorectal cancer tumor, a childhood colorectal cancer tumor, childhood craniopharyngioma tissue, a ductal carcinoma in situ (DCIS), a childhood embryonal tumor, endometrial cancer (uterine cancer) tissue, childhood ependymoma tissue, esophageal cancer tissue, childhood esophageal cancer tissue, esthesioneuroblastoma (head and neck cancer) tissue, a childhood extracranial germ cell tumor, an extragonadal germ cell tumor, eye cancer tissue, an intraocular melanoma, a retinoblastoma, fallopian tube cancer tissue, gallbladder cancer tissue, gastric (stomach) cancer tissue, childhood gastric (stomach) cancer tissue, a gastrointestinal carcinoid tumor, a gastrointestinal stromal tumor (GIST), a childhood gastrointestinal stromal tumor, a germ cell tumor (e.g., a childhood central nervous system germ cell tumor, a childhood extracranial germ cell tumor, an extragonadal germ cell tumor, an ovarian germ cell tumor, or testicular cancer tissue), head and neck cancer tissue, a childhood heart tumor, hepatocellular cancer (HCC) tissue, an islet cell tumor (pancreatic neuroendocrine tumors), kidney or renal cell cancer (RCC) tissue, laryngeal cancer tissue, leukemia, liver cancer tissue, lung cancer (non-small cell and small cell) tissue, childhood lung cancer tissue, male breast cancer tissue, a malignant fibrous histiocytoma of bone and osteosarcoma, a melanoma, a childhood melanoma, an intraocular melanoma, a childhood intraocular melanoma, a merkel cell carcinoma, a malignant mesothelioma, a childhood mesothelioma, metastatic cancer tissue, metastatic squamous neck cancer with occult primary tissue, a midline tract carcinoma with NUT gene changes, mouth cancer (head and neck cancer) tissue, multiple endocrine neoplasia syndrome tissue, a multiple myeloma/plasma cell neoplasm, myelodysplastic syndrome tissue, a myelodysplastic/myeloproliferative neoplasm, a chronic myeloproliferative neoplasm, nasal cavity and paranasal sinus cancer tissue, nasopharyngeal cancer (NPC) tissue, neuroblastoma tissue, non-small cell lung cancer tissue, oral cancer tissue, lip and oral cavity cancer and oropharyngeal cancer tissue, osteosarcoma and malignant fibrous histiocytoma of bone tissue, ovarian cancer tissue, childhood ovarian cancer tissue, pancreatic cancer tissue, childhood pancreatic cancer tissue, papillomatosis (childhood laryngeal) tissue, paraganglioma tissue, childhood paraganglioma tissue, paranasal sinus and nasal cavity cancer tissue, parathyroid cancer tissue, penile cancer tissue, pharyngeal cancer tissue, pheochromocytoma tissue, childhood pheochromocytoma tissue, a pituitary tumor, a plasma cell neoplasm/multiple myeloma, a pleuropulmonary blastoma, a primary central nervous system (CNS) lymphoma, primary peritoneal cancer tissue, prostate cancer tissue, rectal cancer tissue, a retinoblastoma, a childhood rhabdomyosarcoma, salivary gland cancer tissue, a sarcoma (e.g., a childhood vascular tumor, osteosarcoma, uterine sarcoma, etc.), Sezary syndrome (lymphoma) tissue, skin cancer tissue, childhood skin cancer tissue, small cell lung cancer tissue, small intestine cancer tissue, a squamous cell carcinoma of the skin, a squamous neck cancer with occult primary, a cutaneous t-cell lymphoma, testicular cancer tissue, childhood testicular cancer tissue, throat cancer (e.g., nasopharyngeal cancer, oropharyngeal cancer, hypopharyngeal cancer) tissue, a thymoma or thymic carcinoma, thyroid cancer tissue, transitional cell cancer of the renal pelvis and ureter tissue, unknown primary carcinoma tissue, ureter or renal pelvis tissue, transitional cell cancer (kidney (renal cell) cancer tissue, urethral cancer tissue, endometrial uterine cancer tissue, uterine sarcoma tissue, vaginal cancer tissue, childhood vaginal cancer tissue, a vascular tumor, vulvar cancer tissue, a Wilms tumor or other childhood kidney tumor.

In some embodiments, a cell source of any embodiment of the present disclosure is a first cancer. In some such embodiments, the first cancer is a stage of a breast cancer, a stage of a lung cancer, a stage of a prostate cancer, a stage of a colorectal cancer, a stage of a renal cancer, a stage of a uterine cancer, a stage of a pancreatic cancer, a stage of a cancer of the esophagus, a stage of a lymphoma, a stage of a head/neck cancer, a stage of a ovarian cancer, a stage of a hepatobiliary cancer, a stage of a melanoma, a stage of a cervical cancer, a stage of a multiple myeloma, a stage of a leukemia, a stage of a thyroid cancer, a stage of a bladder cancer, or a stage of a gastric cancer.

In some embodiments, a cell source of any embodiment of the present disclosure is a predetermined stage of a breast cancer, a predetermined stage of a lung cancer, a predetermined stage of a prostate cancer, a predetermined stage of a colorectal cancer, a predetermined stage of a renal cancer, a predetermined stage of a uterine cancer, a predetermined stage of a pancreatic cancer, a predetermined stage of a cancer of the esophagus, a predetermined stage of a lymphoma, a predetermined stage of a head/neck cancer, a predetermined stage of a ovarian cancer, a predetermined stage of a hepatobiliary cancer, a predetermined stage of a melanoma, a predetermined stage of a cervical cancer, a predetermined stage of a multiple myeloma, a predetermined stage of a leukemia, a predetermined stage of a thyroid cancer, a predetermined stage of a bladder cancer, or a predetermined stage of a gastric cancer.

In some embodiments, a cell source of any embodiment of the present disclosure is from a non-cancerous tissue. In some embodiments, a cell source of any embodiment of the present disclosure is from cells that derive from healthy tissue. In some embodiments, a cell source of any embodiment of the present disclosure is from a healthy tissue such as breast, lung, prostate, colorectal, renal, uterine, pancreatic, esophageal, lymph, ovarian, cervical, epidermal, thyroid, bladder, gastric, or a combination thereof.

In some embodiments, a cell source of any embodiment of the present disclosure is derived from one tissue type. In some embodiments, a cell source of any embodiment of the present disclosure is derived from two or more tissue types. In some embodiments, a tissue type includes one or more cell types (e.g., a combination of healthy, non-cancerous cells and cancerous cells). In some embodiments, a tissue type includes one cell type (e.g., one of either cancerous or healthy, non-cancerous cells).

In some embodiments, a cell source of any embodiment of the present disclosure constitutes one cell type, two cell types, three cell types, four cell types, five cell types, six cell types, seven cell types, eight cell types, nine cell types, ten cell types, or more than ten cell types.

In some embodiments, a cell source of any embodiment of the present disclosure is liver cells. In some such embodiments, the cell source is hepatocytes, hepatic stellate fat storing cells (ITO cells), Kupffer cells, sinusoidal endothelial cells, or any combination thereof.

In some embodiments, a cell source of any embodiment of the present disclosure is stomach cells. In some such embodiments, the first cell source is parietal cells.

In some embodiments, a cell source of any embodiment of the present disclosure is one or more types of human cells. In some such embodiments, the cell source is adaptive NK cells, adipocytes, alveolar cells, Alzheimer type II astrocytes, amacrine cells, ameloblasts, astrocytes, B cells, basophils, basophil activation cells, basophilia cells, Betz cells, bistratified cells, Boettcher cells, cardiac muscle cells, CD4+ T cells, cementoblasts, cerebellar granule cells, cholangiocytes, cholecystocytes, chromaffin cells, cigar cells, club cells, orticotropic cells, cytotoxic T cells, dendritic cells, enterochromaffin cells, enterochromaffin-like cells, eosinophils, extraglomerular mesangial cells, faggot cells, fat pad cells, gastric chief cells, goblet cells, gonadotropic cells, hepatic stellate cells, hepatocytes, hypersegmented neutrophils, intraglomerular mesangial cells, juxtaglomerular cells, keratinocytes, kidney proximal tubule brush border cells, Kupffer cells, lactotropic cells, Leydig cells, macrophages, macula densa cells, mast cells, megakaryocytes, melanocytes, microfold cells, monocytes, natural killer cells, natural killer T cells, glitter cells, neutrophils, osteoblasts, osteoclasts, osteocytes, oxyphil cells (parathyroid), paneth cells, parafollicular cells, parasol cells, parathyroid chief cells, parietal cells, parvocellular neurosecretory cells, peg cells, pericytes, peritubular myoid cells, platelets, podocytes, regulatory T cell, reticulocytes, retina bipolar cells retina horizontal cells, retinal ganglion cells, retinal precursor cells, sentinel cells, sertoli cells, somatomammotrophic cells, somatotropic cells, stellate cells, sustentacular cells, T cells, T helper cells, telocytes, tendon cells, thyrotropic cells, transitional B cells, trichocytes (human), tuft cells, unipolar brush cells, white blood cells, zellballens, or any combination thereof. In some such embodiments, such cells of the first cell source are healthy. In alternative embodiments such cells of the first cell source are afflicted with cancer.

In some embodiments, a cell source of any embodiment of the present disclosure is any combination of cell types provided that such cell types originated from a single organ. In some such embodiments this single organ is breast, lung, prostate, colon/rectum, kidney, uterus, pancreas, esophagus, blood, head/neck, ovary, liver, cervix, thyroid, bladder, or stomach. In some embodiments this single organ is healthy. In alternative embodiments this single organ is afflicted with cancer that originated in the single organ. In still further alternative embodiments, this single organ is afflicted with cancer that originated in an organ other than the single organ and metastasized to the single organ.

In some embodiments, a cell source of any embodiment of the present disclosure is any combination of cell types provided that such cell types originated from a predetermined set of organs. In some such embodiments this predetermined set of organs is any two organs in the set breast, lung, prostate, colon/rectum, kidney, uterus, pancreas, esophagus, blood, head/neck, ovary, liver, cervix, thyroid, bladder, and stomach. In some embodiments this predetermined set of organs is healthy. In alternative embodiments this predetermined set of organs is afflicted with cancer that originated in one of the organs in the predetermined set of organs. In still further alternative embodiments, the predetermined set of organs is afflicted with cancer that originated in an organ other than the predetermined set of organs and metastasized to the predetermined set of organs.

In some embodiments, a cell source of any embodiment of the present disclosure is any combination of cell types provided that such cell types originated from a predetermined set of organs. In some such embodiments this predetermined set of organs is any three organs in the set breast, lung, prostate, colon/rectum, kidney, uterus, pancreas, esophagus, blood, head/neck, ovary, liver, cervix, thyroid, bladder, and stomach. In some embodiments this predetermined set of organs is healthy. In alternative embodiments this predetermined set of organs is afflicted with cancer that originated in one of the organs in the predetermined set of organs. In still further alternative embodiments, the predetermined set of organs is afflicted with cancer that originated in an organ other than the predetermined set of organs and metastasized to the predetermined set of organs.

In some embodiments, a cell source of any embodiment of the present disclosure is any combination of cell types provided that such cell types originated from a predetermined set of organs. In some such embodiments this predetermined set of organs is any four organs, five organs, six organs, or seven organs in the set breast, lung, prostate, colon/rectum, kidney, uterus, pancreas, esophagus, blood, head/neck, ovary, liver, cervix, thyroid, bladder, and stomach. In some embodiments this predetermined set of organs is healthy. In alternative embodiments this predetermined set of organs is afflicted with cancer that originated in one of the organs in the predetermined set of organs. In still further alternative embodiments, the predetermined set of organs is afflicted with cancer that originated in an organ other than the predetermined set of organs and metastasized to the predetermined set of organs.

In some specific embodiments, a cell source of any embodiment of the present disclosure is white blood cells. In some such embodiments, the cell source is neutrophils, eosinophils, basophils, lymphocytes, B lymphocytes, T lymphocytes, cytotoxic T cells, monocytes, or any combination thereof.

### CONCLUSION

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event (” or “in response to detecting (the stated condition or event),” depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

1. A method of estimating a first cell source fraction in a first biological sample from a test subject of a given species, the method comprising:
at a computer system having one or more processors, and memory storing one or more programs for execution by the one or more processors:
(A) obtaining a methylation state of each nucleic acid fragment in a first plurality of nucleic acid fragments, in electronic form, from a first plurality of cell-free nucleic acid molecules in the first biological sample at a first time period, wherein the first plurality of nucleic fragments is more than 1000 nucleic acid fragments;
(B) individually assigning a first score to each respective nucleic acid fragment in the first plurality of nucleic acid fragments, thereby obtaining a plurality of first scores, wherein:
each respective first score represents a likelihood that the corresponding nucleic acid fragment was obtained from a cell-free nucleic acid molecule that originated from the first cell source,
the individually assigning (B) comprises i) comparing a methylation state of the respective nucleic acid fragment against a first canonical set of methylation state vectors and against a second canonical set of methylation state vectors, or ii) presenting the methylation state of the respective nucleic acid fragment to a first classifier trained at least in part on the first canonical set of methylation state vectors and the second canonical set of methylation state vectors,
each canonical methylation state vector in the first canonical set of methylation state vectors is derived from a respective first tissue sample or a respective first cell-free nucleic acid sample of a corresponding reference subject in a first plurality of reference subjects, wherein the respective first tissue sample or the respective first cell-free nucleic acid sample corresponds to the first cell source,
each canonical methylation state vector in the second canonical set of methylation state vectors is derived from a respective second tissue sample or a respective second cell-free nucleic acid sample of a corresponding reference subject in a second plurality of reference subjects, wherein the respective second tissue sample or the respective second cell-free nucleic acid sample corresponds to a second cell source, wherein the second cell source is other than the first cell source;
(C) transforming the plurality of first scores into a first plurality of counts, wherein:
each count in the first plurality of counts is for a methylation site in a first predetermined set of methylation sites in the genome of a reference sequence of the species, and
the first predetermined set of methylation sites is associated with the first cell source; and
(D) estimating a first instance of the first cell source fraction in the first biological sample using the first plurality of counts by comparing the respective count of each respective methylation site in the first predetermined set of methylation sites represented by the first plurality of counts to a corresponding reference score for the respective methylation site in a first reference set, wherein each corresponding reference score in the first reference set is obtained by determining a frequency of methylation of the corresponding methylation site in nucleic acid fragments obtained from the respective first tissue sample or the respective first cell-free nucleic acid sample of each corresponding reference subject in the first plurality of reference subjects.

2. The method of claim 1, wherein each canonical methylation state vector in the first canonical set of methylation state vectors represents the methylation state across the genome of the corresponding reference subject in the first plurality of reference subjects.

3. (canceled)

4. The method of claim 1, wherein
the first cell source is a type of cancer, and
a canonical methylation state vector in the first canonical set of methylation state vectors is derived from a sample of a tumor of the type of cancer obtained from the corresponding reference subject.

5. The method of claim 1, wherein
the first cell source is a type of cancer,
a canonical methylation state vector in the first set of canonical methylation state vectors is derived from cell-free nucleic acids of a reference biological sample from the corresponding reference subject, and
the cell source fraction for the type of cancer in the reference biological sample in the corresponding reference subject is at least two percent, at least ten percent, or at least twenty percent.

6-7. (canceled)

8. The method of claim 1, wherein the second cell source is one or more cell types that are cancer-free.

9. The method of claim 1, the method further comprising:
(E) obtaining a methylation state of each nucleic acid fragment in a second plurality of nucleic acid fragments, in electronic form, from a second plurality of cell-free nucleic acid molecules in a second biological sample of the test subject at a second time period;
(F) individually assigning a second score to each respective nucleic acid fragment in the second plurality of nucleic acid fragments, thereby obtaining a plurality of second scores, wherein
each respective second score represents a likelihood that the nucleic acid fragment was obtained from a cell-free nucleic acid molecule that originated from the first cell source,
the individually assigning (F) comprises: i) comparing the methylation state of the respective nucleic acid fragment against the first canonical set of methylation state vectors and against the second canonical set of methylation state vectors, or ii) presenting the methylation state of the respective nucleic acid fragment to the first classifier,
(G) transforming the plurality of second scores into a second plurality of counts, wherein,
each respective count in the second plurality of counts is for a methylation site in the first predetermined set of methylation sites in the genome of the reference sequence of the species; and
(H) estimating a second instance of the first cell source fraction in the second biological sample using the second plurality of counts by comparing the respective count of each respective methylation site in the first predetermined set of methylation sites represented by the second plurality of counts to a corresponding reference score for the respective methylation site in the first reference set.

10. (canceled)

11. The method of claim 9, the method further comprising using a difference between the first instance of the first cell source fraction and the second instance of the first cell source fraction as a basis or a partial basis for determining an aggressiveness of a disease condition associated with the first cell source in the test subject.

12. The method of claim 9, the method further comprising using a difference between the first instance of the first cell source fraction and the second instance of the first cell source fraction as a basis or a partial basis for determining a treatment option for a disease condition associated with the first cell source in the test subject.

13. The method of claim 1, wherein the first cell source is a type of cancer and the method further comprises using the first instance of the first cell source fraction as a basis or a partial basis for determining a stage of the type of cancer in the test subject.

14. (canceled)

15. The method of claim 1, wherein the first cell source is a type of cancer and the method further comprises using the first cell source fraction as a basis or a partial basis for determining a treatment option for the cancer in the test subject.

16. The method of claim 1, wherein:
the first canonical set of methylation state vectors is a single consensus methylation state vector of the genome of the species formed from a methylation state of nucleic acids in the respective first tissue sample or the respective cell-free nucleic acid sample across the first plurality of reference subjects, and
the second canonical set of methylation state vectors is a single consensus methylation state vector of the genome of the species formed from a methylation state of nucleic acids in the respective second tissue sample or the second cell-free nucleic acid sample across the second plurality of reference subjects.

17. The method of claim 1, wherein:
the first canonical set of methylation state vectors includes a different consensus methylation state vector of the genome of the species for each respective reference subject in the first plurality of reference subjects formed from a methylation state of nucleic acids in the respective first tissue sample or the respective first cell-free nucleic acid sample of the respective reference subject, and
the second canonical set of methylation state vectors includes a different consensus methylation state vector of the genome of the species for each respective reference subject in the second plurality of reference subjects formed from a methylation state of nucleic acids in the respective second tissue sample or the respective second cell-free nucleic acid sample of the respective reference subject.

18. (canceled)

19. The method of claim 1, wherein:
the first plurality of reference subjects comprises at least one hundred reference subjects, and
the second plurality of reference subjects comprises at least one hundred reference subjects other than the first plurality of reference subjects.

20-21. (canceled)

22. The method of claim 1, wherein:
the individually assigning (B) comprises presenting the methylation state of the respective nucleic acid fragment to the first classifier, and
the first classifier is based on a logistic regression algorithm, a neural network algorithm, a support vector machine algorithm, a Naive Bayes algorithm, a nearest neighbor algorithm, a boosted trees algorithm, a random forest algorithm, a convolutional neural network, a decision tree algorithm a mixture model, or a hidden Markov model.

23-25. (canceled)

26. The method of claim 1, wherein the first predetermined set of methylation sites comprises fifty methylation sites, comprises one hundred methylation sites, or comprises five hundred methylation sites in the genome of the species.

27-28. (canceled)

29. The method of claim 1, wherein the transforming the plurality of first scores into a first plurality of counts (C) comprises, for each respective methylation site in the first predetermined set of methylation sites:
(a) determining a first number of nucleic acid fragments in the first plurality of nucleic acid fragments that (i) map to the respective methylation site and (ii) have a first score satisfying a threshold value;
(b) determining a second number of nucleic acid fragments in the plurality of nucleic acid fragments that (i) map to the respective methylation site and (ii) have a first score satisfying or not satisfying the threshold value; and
(c) assigning the score for the respective methylation site as a quotient of the first number and the second number.

30-31. (canceled)

32. The method of claim 1, wherein each count of each respective methylation site in the first predetermined set of methylation sites is an observed frequency of methylation of the corresponding methylation site across the first plurality of nucleic acid fragments and the estimating (D) comprises:
constructing a Poisson model or a negative binomial distribution assumption using the count of each respective methylation site and the corresponding reference frequency of each respective methylation site in the first reference set;
using the Poisson model or the negative binomial distribution assumption to form a cumulative density function across a range of calculated first cell source fractions; and
deeming the first instance of the first cell source fraction to be a mean of the cumulative density function across the range of calculated first cell source fractions.

33. The method of claim 1, wherein each count of each respective methylation site in the first predetermined set of methylation sites is an observed frequency of methylation of the corresponding methylation site across the first plurality of nucleic acid fragments and the estimating (D) comprises:
constructing a respective Poisson model or a respective negative binomial distribution assumption using the count for each respective methylation site and the corresponding reference frequency of the methylation site in the first reference set, thereby constructing a plurality of Poisson models or a plurality of negative binomial distribution assumptions; and
using each respective Poisson model or each respective negative binomial distribution assumption to form a corresponding cumulative density function across a range of calculated first cell source fractions; and
deeming the first instance of the first cell source fraction to be the mean of the cumulative density function across the range of calculated first cell source fractions combined across the plurality of Poisson models or the plurality of negative binomial distribution assumptions.

34. (canceled)

35. The method of claim 1, wherein the first cell source is (i) a plurality of cells of a first cancer type, (ii) a plurality of cells of a first cancer type at a first stage of the first cancer type, (iii) a plurality of cells of a single cell type, (iv) a plurality of cells of a single tissue type, (v) a plurality of cells originating from a first organ type, wherein the first organ type is afflicted with a cancer originating in the first organ type, (vi) a plurality of cells originating from a first organ type wherein the first organ type is afflicted with a cancer originating from a second organ type, (vii) healthy cells, or (viii) white blood cells.

36. The method of claim 1, wherein the first biological sample comprises blood, whole blood, plasma, serum, urine, cerebrospinal fluid, fecal, saliva, sweat, tears, pleural fluid, pericardial fluid, or peritoneal fluid of the test subject.

37. (canceled)

38. The method of claim 1, wherein the first cell source is a plurality of cells of a first cancer type and wherein the first cancer type is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof.

39-40. (canceled)

41. A computing system, comprising:
one or more processors;
memory storing one or more programs to be executed by the one or more processor, the one or more programs comprising instructions for estimating a first cell source fraction in a first biological sample in a test subject of a given species by a method comprising:
(A) obtaining a methylation state of each nucleic acid fragment in a first plurality of nucleic acid fragments in electronic form from a first plurality of cell-free nucleic acid molecules in the first biological sample at a first time period, wherein the first plurality of nucleic fragments is more than 1000 nucleic acid fragments;
(B) individually assigning a first score to each respective nucleic acid fragment in the first plurality of nucleic acid fragments, thereby obtaining a plurality of first scores, wherein:
each respective first score represents a likelihood that the corresponding nucleic acid fragment was obtained from a cell-free nucleic acid molecule that originated from the first cell source,
the individually assigning (B) comprises i) comparing a methylation state of the respective nucleic acid fragment against a first canonical set of methylation state vectors and against a second canonical set of methylation state vectors representative of a source other than the first cell source, or ii) presenting the methylation state of the respective nucleic acid fragment to a first classifier trained at least in part on the first canonical set of methylation state vectors and the second canonical set of methylation state vectors,
each canonical methylation state vector in the first canonical set of methylation state vectors is derived from a respective first tissue sample or a respective first cell-free nucleic acid sample of a corresponding reference subject in a first plurality of reference subjects, wherein the respective first tissue sample or the respective first cell-free nucleic acid sample corresponds to the first cell source,
each canonical methylation state vector in the second canonical set of methylation state vectors is derived from a respective second tissue sample or a respective second cell-free nucleic acid sample of a corresponding reference subject in a second plurality of reference subjects, wherein the respective second tissue sample or the respective second cell-free nucleic acid sample corresponds to a second cell source;
(C) transforming the plurality of first scores into a first plurality of counts, wherein:
each count in the first plurality of counts is for a methylation site in a first predetermined set of methylation sites in the genome of a reference sequence of the species, and
the first predetermined set of methylation sites is associated with the first cell source; and
(D) estimating a first instance of the first cell source fraction in the first biological sample using the first plurality of counts by comparing the respective count of each respective methylation site in the first predetermined set of methylation sites represented by the first plurality of counts to a corresponding reference score for the respective methylation site in a first reference set, wherein each corresponding reference score in the first reference set is obtained by determining a frequency of methylation of the corresponding methylation site in nucleic acid fragments obtained from the respective first tissue sample or the respective first cell-free nucleic acid sample of a corresponding reference subject in the first plurality of reference subjects.

42. (canceled)

43. A non-transitory computer readable storage medium storing one or more programs for estimating a first cell source fraction in a first biological sample in a test subject of a given species, the one or more programs configured for execution by a computer, wherein the one or more programs comprise instructions for:
(A) obtaining a methylation state of each nucleic acid fragment in a first plurality of nucleic acid fragments in electronic form from a first plurality of cell-free nucleic acid molecules in the first biological sample at a first time period, wherein the first plurality of nucleic fragments is more than 1000 nucleic acid fragments;
(B) individually assigning a first score to each respective nucleic acid fragment in the first plurality of nucleic acid fragments, thereby obtaining a plurality of first scores, wherein:
each respective first score represents a likelihood that the corresponding nucleic acid fragment was obtained from a cell-free nucleic acid molecule that originated from the first cell source,
the individually assigning (B) comprises i) comparing a methylation state of the respective nucleic acid fragment against a first canonical set of methylation state vectors and against a second canonical set of methylation state vectors representative of a source other than the first cell source, or ii) presenting the methylation state of the respective nucleic acid fragment to a first classifier trained at least in part on the first canonical set of methylation state vectors and the second canonical set of methylation state vectors,
each canonical methylation state vector in the first canonical set of methylation state vectors is derived from a respective first tissue sample or a respective first cell-free nucleic acid sample of a corresponding reference subject in a first plurality of reference subjects, wherein the respective first tissue sample or the respective first cell-free nucleic acid sample corresponds to the first cell source,
each canonical methylation state vector in the second canonical set of methylation state vectors is derived from a respective second tissue sample or a respective first cell-free nucleic acid sample of a corresponding reference subject in a second plurality of reference subjects, wherein the respective second tissue sample or the respective second cell-free nucleic acid sample corresponds to a second cell source;
(C) transforming the plurality of first scores into a first plurality of counts, wherein:
each count in the first plurality of counts is for a methylation site in a first predetermined set of methylation sites in the genome of a reference sequence of the species, and
the first predetermined set of methylation sites is associated with the first cell source; and
(D) estimating a first instance of the first cell source fraction in the first biological sample using the first plurality of counts by comparing the respective count of each respective methylation site in the first predetermined set of methylation sites represented by the first plurality of counts to a corresponding reference score for the respective methylation site in a first reference set, wherein each corresponding reference score in the first reference set is obtained by determining a frequency of methylation of the corresponding methylation site in nucleic acid fragments obtained from the respective first tissue sample or the respective first cell-free nucleic acid sample of a corresponding reference subject in the first plurality of reference subjects.

44-137. (canceled)
